SEQUENCE LISTING (23 sequences)

SEQ ID NO: 1 - DNA, 21 bp, Drosophila melanogaster
agccgatgaagaagctctacg

SEQ ID NO: 2 - DNA, 23 bp, Drosophila melanogaster
atgtcgtagacaagcacctggta

SEQ ID NO: 3 - DNA, 23 bp, Drosophila melanogaster
atcgaggaggaaatcgagatgga

SEQ ID NO: 4 - DNA, 23 bp, Drosophila melanogaster
ggaagctatgccactgctgaata

SEQ ID NO: 5 - DNA, 23 bp, Drosophila melanogaster
agatgcagaccctgtacatagac

SEQ ID NO: 6 - DNA, 23 bp, Drosophila melanogaster
ctcgtcgttcaggtggtacagaa

SEQ ID NO: 7 - DNA, 23 bp, Drosophila melanogaster
gtatcgacaacagagtcggtcgc

SEQ ID NO: 8 - DNA, 23 bp, Drosophila melanogaster
ttggtgagcggaccgacagctgc

SEQ ID NO: 9 - protein, 588 aa, Drosophila melanogaster
MRAWLLLLAVLATFQTIVRVASTEDISQRFIAAIAPVAAHIPLASASGSGSGRSGSRSVGASTSTALAKAFNPFSEPASFSDSDKSHRSKTNKKPSKSDANRQFNEVHKPRTDQLENSKNKSKQLVNKPNHNKMAVKEQRSHHKKSHHHRSHQPKQASASTESHQSSSIESIFVEEPTLVLDREVASINVPANAKAIIAEQGPSTYSKEALIKDKLKPDPSTLVEIEKSLLSLFNMKRPPKIDRSKIIIPEPMKKLYAEIMGHELDSVNIPKPGLLTKSANTVRSFTHKDSKIDDRFPHHHRFRLHFDVKSIPADEKLKAAELQLTRDALSQQVVASRSSANRTRYQVLVYDITRVGVRGQREPSYLLLDTKTVRLNSTDTVSLDVQPAVDRWLASPQRNYGLLVEVRTVRSLKPAPHHHVRLRRSADEAHERWQHKQPLLFTYTDDGRHKARSIRDVSGGEGGGKGGRNKRQPRRPTRRKNHDDTCRRHSLYVDFSDVGWDDWIVAPLGYDAYYCHGKCPFPLADHFNSTNHAVVQTLVNNMNPGKVPKACCVPTQLDSVAMLYLNDQSTVVLKNYQEMTVVGCGCR

SEQ ID NO: 10 - protein, 455 aa, Drosophila melanogaster
MSGLRNTSEAVAVLASLGLGMVLLMFVATTPPAVEATQSGIYIDNGKDQTIMHRVLSEDDKLDVSYEILEFLGIAERPTHLSSHQLSLRKSAPKFLLDVYHRITAEEGLSDQDEDDDYERGHRSRRSADLEEDEGEQQKNFITDLDKRAIDESDIIMTFLNKRHHNVDELRHEHGRRLWFDVSNVPNDNYLVMAELRIYQNANEGKWLTANREFTITVYAIGTGTLGQHTMEPLSSVNTTGDYVGWLELNVTEGLHEWLVKSKDNHGIYIGAHAVNRPDREVKLDDIGLIHRKVDDEFQPFMIGFFRGPELIKATAHSSHHRSKRSASHPRKRKKSVSPNNVPLLEPMESTRSCQMQTLYIDFKDLGWHDWIIAPEGYGAFYCSGECNFPLNAHMNATNHAIVQTLVHLLEPKKVPKPCCAPTRLGALPVLYHLNDENVNLKKYRNMIVKSCGCH

SEQ ID NO: 11 - protein, 400 aa, Drosophila melanogaster
MLNVFFLTSLFYAASATTYVTTNNHIEMPIYQKRPLSEQMEMIDILDLGDRPRRQAEPNLHNSASKFLLEVYNEISEDQEPKEVLHQRHKRSLDDDILISNEDRQEIASCNSILTFSSRLKPEQLDNELDMHITFNTNDVPVDLSLVQAMLRIYKQPSLVDRRANFTVSVYRKLDNRQDFSYRILGSVNTTSSQRGWLEFNLTDTLRYWLHNKGLQRRNELRISIGDSQLSTFAAGLVTPQASRTSLEPFIVGYFNGPELLVKIQKLRFKRDLEKRRAGGGSPPPPPPPPVDLYRPPQSCERLNFTVDFKELHMHNWVIAPKKFEAYFCGGGCNFPLGTKMNATNHAIVQTLMHLKQPHLPKPCCVPTVLGAITILRYLNEDIIDLTKYQKAVAKECGCH

SEQ ID NO: 12 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 13 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 14 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 15 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 16 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 17 - protein, 402 aa, Homo sapiens [sequence omitted]

SEQ ID NO: 18 - DNA, 38 bp, Drosophila melanogaster
ctcctgaattcgcagacagcgtggcgtcagcgatttca

SEQ ID NO: 19 - DNA, 42 bp, Drosophila melanogaster
agtgtctggcggcgtagcaagactggcgacattctgtctggt

SEQ ID NO: 20 - DNA, 38 bp, Drosophila melanogaster; identical to SEQ ID NO: 18

SEQ ID NO: 21 - DNA, 38 bp, Drosophila melanogaster; identical to SEQ ID NO: 18

SEQ ID NO: 22 - DNA, 31 bp, Drosophila melanogaster
ctcctgaattcgcagacagcgagcgatttca

SEQ ID NO: 23 - DNA, 27 bp, Drosophila melanogaster
ctcctgaatttggcgtcagcgatttca

(19) United States
(12) Patent Application Publication
(10) Pub. No.: US 2005/0227352 A1
(43) Pub. Date: Oct. 13, 2005

(54) Use of BMP (BAM) signaling to control stem cell fate determination

(75) Inventor: Ting Xie, Leawood, KS (US)

Correspondence Address: POLSINELLI SHALTON WELTE SUELTHAUS P.C., 700 W. 47TH STREET, SUITE 1000, KANSAS CITY, MO, 64112-1802, US

(73) Assignee: Stowers Institute for Medical Research

(21) Appl. No.: 11/021,984

(22) Filed: Dec. 23, 2004

Related U.S. Application Data
(60) Provisional application No. 60/561,024, filed on Apr. 9, 2004.

(51) Int. Cl. C12N 5/06; C12N 5/08

(52) U.S. Cl. 435/348; 435/366

(57) ABSTRACT

The present invention generally provides a means to control stem cell self-renewal and differentiation. More particularly, the current invention is directed toward the elucidation of a signal transduction pathway that controls the expression of a gene necessary for differentiation of stem daughter cells. By modulating the levels of key regulatory molecules within this pathway, the fate of stem cell self-renewal and differentiation may be controlled. In addition, the present invention also provides mutant organisms, tissues, cells, as well methods where the level of key molecules within the pathway are modulated.

### CROSS-REFERENCE TO RELATED APPLICATION

This application claims priority from Provisional Application Ser. No. 60/561,024 filed on Apr. 9, 2004, which is hereby incorporated by reference in its entirety.

### FIELD OF THE INVENTION

The present invention relates to the use of signal transduction pathways to control stem cell self renewal, differentiation and proliferation. More particularly, the invention relates to the use of BMP signal transduction pathways to modulate germline stem cell self renewal, differentiation and proliferation.

### BACKGROUND OF THE INVENTION

Stem cells in adult tissues have the ability to self-renew and generate differentiated cells that maintain tissue homeostasis. Specific regulatory microenvironments, also known as niches, are thought to regulate many stem cell types by producing signals important for stem cell proliferation and differentiation (Watt and Hogan, 2000; Spradling et al., 2001). Stem cells usually divide asymmetrically to generate parent stem cells and differentiated cells, while they can also undergo symmetric cell division to replenish lost stem cells or expand the stem cell pool (Xie and Spradling, 2000; Zhu and Xie, 2003). In some tissues that undergo quick cell turnover, stem cells are relatively active, whereas they remain largely quiescent in the tissues where cell turnover is rare.

But it remains unclear how niche signals regulate self-renewal versus differentiation and quiescence versus proliferation. Niche signals must up-regulate genes in stem cells that are important for maintaining the undifferentiated state and promoting self-renewal. At the same time, the signals must repress genes that can cause stem cells to differentiate. Even though many niche signals have been identified for different stem cell types in many systems, it is still largely unknown how niche signals control stem cell self-renewal and differentiation.

The Drosophila ovarian germline stem cells (GSCs) have become an attractive system to study stem cells and their relationship to niches (Xie and Spradling, 2001; Lin, 2002). Two or three GSCs are located at the tip of the ovariole, also known as the germarium, and are surrounded by terminal filament cells, cap cells, and inner sheath cells that form a niche for GSCs. GSCs and their progeny in the germarium can be reliably identified and distinguished by a germ cell-specific, vesicle-rich structure, called the fusome, which is rich in membrane skeletal proteins, such as hu-li tai shao (Hts) and α-spectrin (Lin et al., 1994; de Cuevas et al., 1997). In GSCs and their immediate differentiating daughter cells, cystoblasts, the fusome is spherical in shape, the fusome is also known as the spectrosome. GSCs are invariably anchored to cap cells through adherens junctions (Song et al., 2002). Upon GSC division, the original GSC remains anchored to cap cells and retains stem cell identity, while the cystoblast moves away from cap cells and undergoes differentiation. A cystoblast will undergo synchronous mitotic divisions to generate two-, four-, eight-, and sixteen-cell cysts, in which the fusome is branched to interconnect individual cystocytes (Lin et al., 1994). Similarly, in the Drosophila testis, hub cells in the tip of the testis form a niche supporting spermatogonial GSCs (Tulina and Matunis, 2001; Kiger et al., 2001).

In GSCs, the interplay between genes involved in self-renewal versus differentiation remains unclear. Previous studies have demonstrated that direct contact with cap cells is necessary for maintaining GSC identity (Xie and Spradling, 2000; Song et al., 2002). As a GSC is lost, a neighboring GSC can generate two daughter cells that both contact cap cells and remain as GSCs, thus replenishing a vacant niche space. Loss of adherens junctions between cap cells and GSCs also leads GSCs to retreat away from cap cells and undergo differentiation. It is known that terminal filament/cap cells express piwi, dpp, fs(l)Yb (Yb), and hedgehog (hh) that are essential for maintaining GSC asymmetric cell division (King et al., 2001; Cox et al., 2000; King and Lin, 1999; Xie and Spradling, 1998, 2000). Mutations in these genes disrupt asymmetric division, resulting in GSC loss, while overexpression of these genes slows down or completely prevents GSC differentiation. Intrinsic factors in GSCs, including pumillio, nanos, dpp receptors and downstream components, are also important for GSC maintenance (Forbes and Lehmann, 1998; Lin and Spradling, 1997; Xie and Spradling, 1998). Mutations in these genes also affect the normal GSC asymmetric division and result in producing only cystoblasts, causing GSC loss. Two intrinsic factors, bag of marbles (bam) and benign germ cell neoplasia (bgcn) are required in cystoblasts for their proper differentiation (Lavoie et al., 1999, McKearin and Ohlstein, 1995; McKearin and Spradling, 1990). Mutations in bam and bgcn prevent cystoblasts from differentiating, causing the accumulation of undifferentiated germ cells.

The role that niche signaling plays in the regulation of genes involved in stem cell proliferation and differentiation also remain largely unknown. It has been shown that a niche signal belonging to the BMP (Bone Morphogenetic Protein) family, dpp, is somehow associated with the regulation of bam expression. But the precise role that dpp signaling plays in the regulation of bam expression has not been characterized. In fact, the functions of dpp signaling and bam in the maintenance of GSCs and differentiation of cystoblasts seem to be directly opposing. Loss of bam function completely eliminates cystoblast differentiation, similar to that caused by dpp overexpression (McKearin and Spradling, 1990; Xie and Spradling, 1998). Forced overexpression of bam in GSCs causes their elimination, similar to that observed when dpp signaling is disrupted in GSCs (Ohlstein and McKearin, 1997; Xie and Spradling, 1998). Based upon these observations, it seems that ban is both necessary and sufficient for cystoblast differentiation just as dpp signaling is for GSC maintenance.

Several studies have supported this working model (Xie and Spradling, 1998; Chen and McKearin, 2003; Kai and Spradling, 2003). bam mRNAs are absent in GSCs and quickly accumulate in cystoblasts and mitotic cysts (McKearin and Spradling, 1990). Overexpression of dpp completely suppresses the expression of BamC protein in germ cells, thus preventing cystoblasts from differentiating (Xie and Spradling, 1998). A recent study by Kai and Spradling (2003) showed that dpp signaling activity is restricted to GSCs and cystoblasts. The asymmetric distribution of bam between GSCs and cystoblasts could be due to transcriptional regulation and/or mRNA stability. The recent elegant bam promoter analysis has revealed that its transcription is actively repressed through a silencer in its 5′ UTR (Chen and McKearin, 2003). But whether and how dpp signaling directly represses ban transcription remains unknown.

A need, therefore, exists to reveal a link between niche signals and their role in regulating genes that are essential for stem cell self-renewal and differentiation. Elucidating this link is essential not only for understanding how stem cell behavior is controlled, but also for using stem cells in regenerative medicine.

### SUMMARY OF THE INVENTION

Briefly, a direct connection has been discovered between niche signals and repression of differentiation genes in stem cells. The present discovery, therefore, generally provides methods and compositions that may be employed to control stem cell self-renewal and differentiation. More particularly, the current invention is directed toward the elucidation of a signal transduction pathway that controls the expression of a gene necessary for differentiation of stem daughter cells. By modulating the levels of key regulatory molecules within this pathway, the fate of stem cell self-renewal and differentiation may be controlled.

One aspect of the invention encompasses a method to maintain the undifferentiated state of a stem cell. In this embodiment, an isolated stem cell or an isolated population of stem cells is contacted with an isolated stem niche cell or a population of stem niche cells. While the two cell types may be contacted either in vivo or in vitro, preferably they are contacted in vitro, such as in culture. Upon contact of the stem cell with the stem niche cell, a molecule expressed from the stem niche cell activates a signal transduction cascade in the stem cell such that the signal transduction cascade directly causes repression of a gene in the stem cell necessary for differentiation of a stem daughter cell.

In yet another aspect of the invention provides a method to maintain the undifferentiated state of an isolated stem cell. The method comprises providing an isolated stem cell and contacting in vitro, the stem cell with an isolated stem niche cell that expresses a BMP polypeptide. Upon contact, the BMP polypeptide activates a signal transduction cascade in the stem cell, wherein the signal transduction cascade directly causes repression of bam transcription in the stem cell.

Another aspect of the invention encompasses a method to maintain the undifferentiated state of an isolated Drosophila germline stem cell. In the method, an isolated Drosophila germline stem cell is contacted in vitro, with an isolated gbb polypeptide such that the gbb polypeptide activates a signal transduction cascade in the germline stem cell. Activation of the signal transduction cascade directly causes repression of bam transcription in the germline stem cell.

A further aspect of the invention relates to a method to repress the expression of bam in an isolated stem cell. The method comprises contacting in vitro, the stem cell with an isolated BMP polypeptide such that the BMP polypeptide activates a signal transduction cascade in the stem cell. Activation of the signal transduction cascade directly causes repression of bam transcription.

In still another aspect of the invention is provided a method to promote the differentiation of a germline stem cell. The method comprises mutating at least one nucleic acid sequence encoding a participant in BMP signaling in the germline stem cell, such that the mutation results in the expression of bam in the germline stem cell.

Yet a further aspect of the invention involves a method to promote the differentiation of a germline stem cell. In this embodiment, the method comprises contacting a BMP polypeptide antagonist or a BMP receptor antagonist with the germline stem cell, wherein the BMP polypeptide antagonist or the BMP receptor antagonist substantially inhibits interaction of a BMP polypeptide with a BMP receptor.

Another aspect of the invention provides a method to promote the differentiation of a germline stem cell. The method comprises introducing a vector into the germline stem cell, where the vector comprises a nucleic acid sequence encoding bam, such that introduction of the vector results in an increase in the expression of bam relative to a wild type germ line stem cell.

An additional aspect of the invention encompasses a method to regulate the expression of a germline stem cell. In this embodiment, the method comprises introducing a vector into the germline stem cell, wherein the vector has a nucleic acid sequence encoding bam operably linked to an inducible promoter.

A further aspect of the invention involves a method to regulate a germline stem cell differentiation pathway. In this embodiment, the pathway is a BMP signaling pathway and the method comprises introducing a vector into the germline stem cell, wherein the vector has a nucleic acid sequence encoding bam operably linked to an inducible promoter.

In yet another aspect of the invention is provided a germline stem cell population having germline stem cells that have been mutagenized such that the germline stem cells are non responsive to a gbb polypeptide. Because the germline cells are non responsive to a gbb polypeptide, bam is expressed by a substantial number of germline stem cells in the population.

An additional aspect of the invention relates to a germline cell population. In this embodiment, the germline cell population comprises germline stem cells and germline stem niche cells. A vector is introduced into the germline stem niche cells, wherein the vector comprises a nucleic acid encoding a gbb polypeptide operatively linked to an inducible promoter. The germline stem niche cells overexpress the gbb polypeptide when the promoter is induced, thereby resulting in the repression of bam transcription by a substantial number of germline stem cells in the population.

In still another aspect of the invention is provided a germline cell population. In this embodiment, the germline cell population comprises germline stem cells and germline stem niche cells. In addition, the germline stem niche cells have a gbb nucleic acid sequence that has been mutagenized such that the expressed gbb polypeptide is inhibited from interacting with a BMP receptor on the germline stem cells, thereby resulting in the expression of bam by a substantial number of germline stem cells in the population.

A further aspect of the invention encompasses in vivo testicular tissue comprising gbb mutant clonal stem niche cells. Typically, the stem niche cells express substantially more gbb polypeptide compared to a stem niche cell in wild type testicular tissue.

Yet another aspect of the invention provides in vivo testicular tissue comprising gbb mutant clonal stem niche cells. In general, the stem niche cells express substantially less gbb polypeptide compared to a stem niche cell in wild type testicular tissue.

Another aspect of the invention involves a mutant Drosophila fly. The mutant fly in this embodiment typically comprises a clonal population of testicular stem niche cells, wherein gbb nucleic acid sequence has been mutagenized in the stem niche cells. As a result of this mutagenasis, there is a decreased population of testicular stem cells in the mutant fly compared to wild type testicular tissue.

A further aspect of the invention relates to another mutant Drosophila fly. The mutant fly in this embodiment generally comprises a clonal population of testicular niche stem cells, wherein gbb nucleic acid sequence is overexpressed. As a result of gbb overexpression, there is an increased population of testicular stem cells in the mutant fly compared to wild type testicular tissue.

An additional aspect of the invention provides yet another mutant Drosophila fly. In this embodiment, the mutant fly comprises a clonal population of germline stem cells that have been mutagenized such that the cells are non responsive to a gbb polypeptide. Because the germline stem cells are non responsive to gbb, bam is expressed and there is a decreased population of testicular stem cells in the mutant fly compared to wild type testicular tissue.

Still another aspect of the invention provides an in vitro germline stem cell cultivation system. The cultivation system in this embodiment comprises isolated germline tissue, wherein the tissue has germline stem cells that have been mutagenized such that the germline stem cells are non responsive to a gbb polypeptide; and a culture medium.

An additional aspect of the invention provides a further in vitro germline stem cell cultivation system. In this embodiment the cultivation system comprises isolated germline tissue, wherein the tissue comprises stem cells and stem niche cells and wherein the stem niche cells have been mutated so that they express substantially more gbb polypeptide compared to stem niche cells in wild type germline tissue; and a culture medium.

A further aspect of the invention provides yet another in vitro germline stem cell cultivation system. In this embodiment, the cultivation system comprises isolated germline tissue, wherein the tissue comprises stem cells and stem niche cells, and wherein the stem niche cells have been mutated so that they express substantially less gbb polypeptide compared to stem niche cells in wild type germline tissue; and a culture medium.

An additional aspect of the invention relates to a marker to detect germline stem cell differentiation. In one embodiment, the marker is a gbb polypeptide.

Other objects an features of the invention will be in part apparent and in part pointed out hereinafter.

### BRIEF DESCRIPTION OF THE DRAWINGS

FIG. 1 illustrates Dpp and Gbb function cooperatively to maintain GSCs in the Drosophila testis; FIGS. 1A-1F, all the images are shown at the same scale and the bar in FIG. 1B represents 10 μm;

FIG. 1A is a diagram of the testis tip, including GSCs and SSCs. Normally, seven to ten GSCs (three shown here for the purpose of demonstration; red round cells) and somatic stem cells (also known as cyst progenitor cells; red elliptic cells) directly contact the hub cells (gray cells); the gonialblast, which is encapsulated by two differentiated somatic cyst cells, moves away from the hub cells and divides to produce a two-cell, four-cell, eight-cell, or sixteen-cell cluster, which can be identified by the branched fusome (green lines);

FIGS. 1B-1E shows testes, which are labeled for FasIII (red, hub cells), Hts (green, spectrosomes and fusomes), and DAPI (blue); the hub cells are labeled red by FasIII. GSCs are highlighted by broken lines; FIG. 1B shows the tip of a wild-type testis showing seven GSCs that contact the hub cells, while FIGS. 1C and 1D show the tips of two gbb⁴/gbb^{D20} mutant testes with two remaining GSCs, as shown in FIG. 1C, or no GSCs, as shown in FIG. 1D, close to the hub cells; FIG. 1E is a tip of dpp^{hr4}/dpp^{hr56} mutant testis showing seven GSCs near hub cells;

FIG. 1F shows a testis labeled for Hts (green) and DAPI (blue); the hub cells are highlighted by a circle, and GSCs are highlighted by broken lines, with the tip of a dpp^{hr4}/dpp^{hr56}; gbb^{D20}/+ mutant testis showing three remaining GSCs near the hub cells;

FIG. 2 shows that punt is required for maintaining GSCs in the Drosophila testis; all the testes are punt¹⁰⁴⁶⁰/punt¹³⁵ mutants and are labeled for Hts (green) and DAPI (blue), except that nuclei of dying cells are labeled red in FIG. 2D; the hub cells are highlighted by circles, whereas the GSCs are identified by broken lines; all the images shown are at the same scale, the bar in FIG. 2A represents 10 μm;

FIG. 2A shows the tip of a punt mutant testis one week after being cultured at 22° C., showing eight GSCs;

In FIGS. 2B and 2C, the tips of two punt mutant testis tips are shown one week after being cultured at 29° C., showing one remaining GSC in FIG. 2B, or no GSCs in FIG. 2C;

FIG. 2D shows the tip of a mutant punt testis tip four days after being cultured at 29° C., showing that no GSCs undergo apoptosis, except for a few late somatic cyst cells (indicated by the arrows);

FIG. 3 illustrates that GSCs and gonialblasts, but not other differentiated germ cells, are responsive to BMP signaling in the testis; the hub cells and the GSCs in FIGS. 3A, 3B, and 3D-3F are highlighted by circles and broken lines, respectively; all the images are shown at the same scale and the bar in FIG. 3A represents 10 μm;

FIG. 3A shows the tip of a Dad-lacZ/+ testis labeled for nuclear lacZ (red), Hts (green) and DNA (blue), showing that all the five GSCs and all the gonialblasts (arrowhead) express Dad; arrows indicate that the somatic cyst cells also express Dad;

FIG. 3B is the tip of a gbb⁴/gbb^{D20}; Dad-lacZ/+ testis labeled for nuclear lacZ (red), Hts (green) and DNA (blue), showing that three remaining GSCs and all the gonialblasts do not have detectable Dad expression;

FIG. 3C is the tip of a testis overexpressing Dad specifically in the germ cells labeled for Hts (green) and DAPI (blue), showing no germ cells in the testis.

FIG. 3D is the tip of a wild-type testis labeled for pMad (red), Hts (green), and DAPI (blue), showing pMad accumulation predominantly in GSCs;

FIG. 3E is the tip of a gbb⁴/gbb^{D4} mutant testis labeled for pMad (red), Hts (green), and DAPI (blue), showing no detectable pMad accumulation in GSCs;

FIG. 3F is the tip of a punt¹⁰⁴⁶⁰/punt¹³⁵ mutant testis labeled for pMad (red), Hts (green), and DAPI (blue), showing no detectable pMad accumulation in the remaining GSC;

FIG. 4 illustrates BMP downstream components which are required in GSCs for their maintenance in the Drosophila testis; all the testes are labeled for lacZ (red), Hts (green) and DNA (blue); marked wild-type or mutant GSCs (highlighted by broken lines) are identified as the cells that contain a spectrosome, directly contact hub cells and lack lacZ expression, whereas unmarked GSCs are lacZ-positive (red); all images are shown at the same scale and the bar in FIG. 4A represents 10 μm;

FIG. 4A is the tip of a testis carrying a marked two-day old wild-type GSC;

FIG. 4B is the tip of a testis carrying two marked two-week old GSCs;

FIG. 4C is the tip of a testis carrying a marked two-day old tkv mutant GSC;

FIG. 4D is a testis that has lost a marked tkv GSC clone two weeks after clone induction;

FIG. 4E is the tip of the testis carrying a marked two-day old mad mutant GSC;

FIG. 4F is the tip of a testis that has lost a marked mad GSC clone two weeks after clone induction;

FIG. 5 illustrates Gbb signaling, which is essential for repressing bam transcription in GSCs in the Drosophila testis; FIGS. 5A-5C are labeled for GFP (green), Hts (red), and DNA (blue), the testis in FIG. 5D is labeled for lacZ (red), GFP (green), Hts (white), and DNA (blue), and the testes in FIGS. 5E and 5F are labeled for Hts (green) and DAPI (blue); the hub cells are highlighted by circles, whereas some GSCs are highlighted by broken lines; all the images are shown at the same scale and the bar in FIG. 5A represents 10 μm;

FIG. 5A is the tip of a Bam-GFP wild-type testis showing no Bam expression in GSCs (arrowhead) and gonialblasts (arrow);

FIG. 5B is the tip of a gbb⁴/gbb^{D20} mutant testis (after being cultured at 29° C. for a week) showing elevated bam-GFP expression in GSCs;

FIG. 5C is the tip of a punt¹⁰⁴⁶/punt¹³⁵ mutant testis (after being cultured at 29° C. for a week), showing elevated bam-GFP expression in GSCs;

FIG. 5D shows the tip of a testis carrying a marked mutant Med GSC (arrowhead, lacZ-negative) and unmarked wild-type GSCs (one by an arrow, lacZ-positive), showing elevated Bam-GFP expression in the mutant Med GSC;

FIG. 5E is the tip of a hs-bam testis showing three remaining GSCs one week after heat-shock treatments;

FIG. 5F shows the tip of a nanos-gal4VP16; UAS-bam testis showing no GSCs;

FIG. 6 illustrates overexpression of Dpp but not Gbb completely repressing bam transcription in the testis; the testes in FIGS. 6A and 6B are labeled for FasIII (red), Hts (green) and DNA (blue), whereas the testes in FIGS. 6C and 6D are labeled for GFP (green), Hts (red), and DAPI (blue); the hub cells are identified by FasIII staining (red) in FIGS. 6A and 6B, and are highlighted by circles in FIGS. 6C and 6D; all the images are shown at the same scale and the bar in FIG. 6A represents 10 μm;

FIG. 6A is the tip of a dpp-overexpressing testis showing more hub cells and slightly more single germ cells with a spectrosome (arrows) two or three cells away from the hub cells;

FIG. 6B is the tip of a gbb-overexpressing testis showing a normal hub and normal germ cell development;

FIG. 6C is the tip of a dpp-overexpressing testis showing that late differentiated germ cells (a two-cell cluster indicated by an arrow; a 16-cell cluster indicated by an arrowhead) fail to express bam-GFP;

FIG. 6D is the tip of a gbb-overexpressing testis showing that bam-GFP expression in the two-cell clusters (arrows) is delayed, but is expressed in late differentiated germ cells (arrowhead);

FIG. 7 illustrates two BMP-like molecules, Dpp and Gbb, are expressed in the somatic cells in the Drosophila testis, the testes in FIGS. 7A-7C are labeled for Hts (red), GFP (green), and DAPI (blue), and their hub cells are highlighted by circles; the images shown in FIGS. 7A-7C are at the same size, and the bar in FIG. 7A represents 10 μm;

FIG. 7A is the tip of a upd-gal4; UAS-GFP testis showing GFP-labeled hub cells;

FIG. 7B is the tip of a c587-gal4; UAS-GFP testis showing GFP-labeled somatic stem cells and somatic cyst cells;

FIG. 7C is the tip of a vasa-GFP testis showing GFP-labeled germ cells including GSCs;

FIG. 7D is a DNA gel with RT-PCR products showing that gbb and dpp mRNAs are primarily present in the somatic cells of the testis, in this gel, mRNAs from the whole testes, purified germ cells, somatic cyst cells/somatic stem cells and hub cells are marked as templates 1, 2, 3, and 4, respectively, vasa serves as a positive control, while rp49 is an internal control. For dpp (10×), approximately 10-fold more RNA templates were used due to its low abundance;

FIG. 8 illustrates a current working model for how BMP signals maintain GSCs in the Drosophila testis; in this model Upd from hub cells, Gbb/Dpp from hub cells/somatic stem cells and an unknown signal initiated by EGFR/Raf signaling from somatic stem cells are important for GSC maintenance, Dpp/Gbb signaling also helps repress bam expression in GSCs and gonialblasts (GBs). Two-cell germ cell cluster distant from hub cells/somatic stem cells receives less BMP signaling and begins to express bam and promote further differentiation.

FIG. 9. shows dpp signaling activity which is restricted to GSCs and some cystoblasts where bam transcription is actively repressed; in FIGS. 9B-9F, cap cells are highlighted by circles, whereas GSCs are indicated by asterisks;

FIG. 9A is an explanatory diagram showing GSCs, their differentiated progeny and surrounding somatic cells;

FIG. 9B is a tip of the Dad-lacZ germarium labeled for nuclear LacZ (red), Hts (green, fusomes), and DAPI (blue), showing high Dad expression in GSCs and a cystoblast (arrow) but not in another cystoblast (arrowhead);

FIG. 9C shows a tip of the bam-GFP germarium labeled for GFP (green), Hts (red, fusomes), and DAPI (blue), showing that bam is transcribed in a cystoblast (arrowhead) and cysts but not in GSCs;

FIG. 9D is a tip of the bam-GFP, Dad-lacZ germarium labeled for LacZ (red), GFP (green), and DAPI (blue), showing that GSCs and two cystoblasts (arrows) express high Dad but no bam, and that a cystoblast (arrowhead) has low Dad and begins to express bam;

FIGS. 9E and 9F show a tip of the bam-GFP germarium labeled for pMad (red), GFP (green), Hts (blue, fusomes) and DAPI (white, FIG. 9F), showing high pMad accumulation in GSCs in which bam is not transcribed and low pMad in a cystoblast (arrowhead, FIG. 9E) in which bam starts to be expressed. The following abbreviations were used: TF, terminal filament; GSCs, germline stem cells; SS, spectrosome; Cpc, cap cells; CB, cystoblast; FS, fusome; IGS, inner sheath cells; CS, cysts; all micrographs are shown at the same scale of 10 μm.

FIG. 10 illustrates dpp, which is essential for repressing bam transcription in GSCs; the germaria in FIGS. 10A-10D are labeled for Hts (red, fusomes), GFP (green), and DAPI (blue), while the germaria in FIGS. 10E-10H are labeled for pMad (red), GFP (green), Hts (blue, fusomes), and DAPI (white, in FIGS. 10F and 10H); all the GSCs are indicated by asterisks, and cap cells in all the panels are marked by circles;

FIG. 10A is a germarial tip from a bam-GFP female cultured at 29° C. for one week, showing no bam expression in GSCs;

FIG. 10B is a germarial tip from a bam-GFP dpp^{hr56}/dpp^{hr4} female cultured at 29° C. for two days, showing that one of two GSCs begins to express bam;

FIGS. 10C and 10D show germarial tips from bam-GFP dpp^{hr56}/dpp^{hr4} females cultured at 29° C. for four days, in FIG. 10C, and one week, in FIG. 10D, showing that the only remaining GSC starts to express bam;

FIGS. 10E and 10F show a germarial tip from a bam-GFP female cultured at 29° C. for four days, showing high pMad accumulation and no bam expression in GSCs;

FIGS. 10G and 10H show a germarial tip from a bam-GFP dpp^{hr56}/dpp^{hr4} female cultured at 29° C. for four days, showing that two mutant GSCs have low pMad and begin to express bam; all micrographs are shown at the same scale of 10 μm;

FIG. 11 shows dpp overexpression is sufficient for repressing bam transcription in single germ cells; germaria in FIG. 11A and FIGS. 11C-11F are labeled for Hts (red, fusomes), GFP (green), and DAPI (blue, while the germarium in FIG. 11B is labeled for vasa (red, germ cells) and Hts (green, fusomes); circles highlight cap cells and asterisks indicate GSCs;

FIG. 11A is a germarial tip showing c587-gal4-driven UAS-GFP expression in inner sheath cells but not in cap cells;

FIG. 11B is a c587-gal4; UAS-dpp germarium resulting from dpp overexpression is filled with single germ cells with a spectrosome; the insert shows the tip of the germarium highlighted by a rectangle in FIG. 11B at a higher magnification (4×), containing only germ cells with a spectrosome (arrows);

FIG. 11C is a tip of the c587-gal4, bam-GFP, UAS-dpp germarium showing that the accumulated spectrosome-containing germ cells (two indicated by arrows) a few cells away from the tip of the germarium fail to express bam-GFP;

FIG. 11D is a middle portion of the c587-gal4, bam-GFP, UAS-dpp germarium showing that spectrosome-containing germ cells (two indicated by arrows) fail to express bam-GFP;

FIG. 11E shows a tip of the hs-gal4, UAS-dpp germarium showing no bam expression in GSCs but its expression in differentiated germ cells without any heat-shock treatments;

FIG. 11F is a tip of the hs-gal4, UAS-dpp germarium showing no bam expression in GSCs and in spectrosome-containing germ cells (two indicated by arrows) distant from the tip after three days of heat-shock treatments; the micrographs in FIGS. 11A and 11C-11F are shown at the same scale of 10 μm, and the bar in FIG. 11B represents 60 μm.

FIG. 12 shows gbb, which is expressed in the somatic cells of the germarium and is essential for maintaining GSCs and for repressing bam transcription in GSCs; germariain FIGS. 12B-12E are labeled for Hts (red, fusomes) and DAPI (blue), while germaria in FIGS. 12F-12I are labeled for Hts (red, fusomes), GFP (green), and DAPI (blue); circles highlight cap cells, whereas asterisks indicate GSCs;

FIG. 12A is a DNA gel with RT-PCR products showing that gbb is expressed in the somatic cells of the germarium but not in GSCs; in this gel, mRNAs for the whole ovaries, agametic ovaries, inner sheath cells and GSC-like germ cells are marked by templates 1, 2, 3, and 4, respectively; vasa and dpp genes are positive controls, while rp49 is an internal control;

FIG. 12B is a germarial tip from a wild-type bam-GFP female cultured at 29° C. for one week showing two GSCs;

FIGS. 12C-12E are germarial tips from the bam-GFP gbb⁴/gbb^{D4} females cultured at 29° C. for one week showing one GSC in FIG. 12C, no GSC but 16-cell cysts (one indicated by arrow in FIG. 12D, and no GSCs and no cysts in FIG. 12E;

FIG. 12F is a germarial tip from a bam-GFP gbb4/Gbb^{D4} female cultured at room temperature for two days showing that one remaining GSC does not express bam;

FIGS. 12G and 12H are germarial tips from bam-GFP gbb⁴/Gbb^{D4} (FIG. 12G) and bam-GFP gbb⁴/gbb^{D20} (FIG. 4H) females cultured at 29° C. for one week showing that the remaining single GSC expresses bam;

FIG. 12I is a germarial tip from a c587-gal4/UAS-gbb, gam-GFP female showing a normal number of GSCs and normal bam-GFP expression in cystoblasts (arrow) and other differentiated germ cells; the micrographs in FIGS. 12B-12I are shown at the same scale, and the bar in FIG. 12B represents 10 μm;

FIG. 13 shows reduction of pMad is correlated with up-regulated bam transcription in gbb mutant GSCs, all the germaria are labeled for pMad (red), GFP (green), Hts (blue, fusomes), and DAPI (white). FIGS. 13A′-13F′ represent the corresponding DAPI images for FIGS. 13a-13F; circles highlight cap cells, while asterisks indicate GSCs;

FIG. 13A is a germarial tip from a bam-GFP female cultured at 29° C. for four days showing normal pMad expression and no bam-GFP expression in GSCs;

FIGS. 13B-13F show germarial tips from either bam-GFP gbb⁴/gbb^{D4} (FIGS. 13C and 13F) or bam-GFP gbb⁴/gbb^{D20} (FIGS. 13B, 13D, and 13E) females cultured at 29° C. for four days, showing reduced pMad expression; gbb mutant GSCs with easily detected pMad do not express bam-GFP (FIGS. 13B and 13C, and one indicated by an arrow in 5D), while the other GSCs with severely reduced pMad show bam-GFP expression (one indicated by an arrowhead in FIGS. 13D, 13E, and 13F), all the micrographs are shown at the same scale of 10 μm;

FIG. 14 shows that punt and Med are required cell-autonomously in GSCs to repress bam transcription, the germarium in FIG. 14A comes from a pun¹⁰⁴⁶⁰/punt¹³⁵ mutant female cultured at 18° C. for two days, and the germaria in FIGS. 14B-14D are from punt¹⁰⁴⁶⁰/punt¹³⁵ mutant females cultured at 29° C. for two days, in FIG. 14B, and one week, shown in FIGS. 14C, and 14D; germaria in FIGS. 14A-14D are labeled for Hts (red, fusomes and somatic follicle cells), GFP (green), and DAPI (blue), germaria in FIGS. 14E-14H from the punt¹⁰⁴⁶⁰/punt¹³⁵ mutant females cultured at 29° C. for four days are labeled for pMad (red), GFP (green), Hts (blue, fusomes), and DAPI (white); germaria in FIGS. 14I-14L are labeled for LacZ (red), GFP (green), Hts (blue), DAPI (white); FIGS. 14F, 14H, 14J, and 14L represent corresponding DAPI stainings for FIGS. 14E, 14G, 14I, and 14K, respectively; circles highlight cap cells, while asterisks indicate GSCs;

FIGS. 14A-14D show germarial tips showing two GFP-negative GSCs, as in FIG. 14A, one GFP-positive and one GFP-negative GSCs, as in FIG. 14B, one GFP-positive GSC, as in FIG. 14C, and no GSC, shown in FIGS. 14D;

FIGS. 14E-14H show germarial tips showing two GFP-positive GSCs with severely reduced pMad, as in FIGS. 14E and 14F, and one GFP-positive GSC with severely reduced pMad, as in FIGS. 14G and 14H;

FIGS. 14I-14L show germarial tips showing a bam-GFP-positive marked punt¹³⁵ GSC (outlined by a dashed line, as in FIG. 14I, and a bam-GFP-positive marked Me²⁶ GSC, as shown by the dashed line in FIG. 14K; all the micrographs are shown at the same scale of 10 μm;

FIG. 15 shows Mad and Med binding directly to the bam silencer in vitro;

FIG. 15A is the sequence alignment of the bipartite bam silencer element and the brk silencer; sites A and B of the bam silencer are designated red and green, respectively, as described by Chen and McKearin, 2003; conserved base pairs between bam and brk silencers are boxed;

FIG. 15B is a Cy5 5′-modified oligonucleotide containing the bipartite bam silencer which was used as a probe in electrophoretic mobility shift assays, to determine specificity of binding, three different unlabeled competitor oligonucleotides were used in 100-fold molar excess: competitor A+B, same sequence without Cy5 5′-modification; competitor A, same flanking sequences with only site A, competitor B, same flanking sequences with only site B;

FIG. 15C shows the immunoblot analysis of purified recombinant GST-tagged proteins with a mouse anti-GST antibody; lane 1, 50 ng of GST; lane 2, 200 ng of GST-Mad; lane 3, 200 ng GST-Med; the 30 kDa band in lane 3 was most likely a C-terminal degradation product of GST-Med protein;

FIG. 15D is a gel shift assay showing Mad or Med bind to the bam silencer in vitro; approximately 10 mol of protein were used in each binding reaction; the double shift bands for Med may result from partially degraded proteins; the labeled probe without protein (lane 1) or with GST protein (lane 2, serve as negative controls; the labeled probe with A and B sites bind to Mad (lane 3) and Med (lane 7); the unlabeled full-length bam silencer (lane 4), site A (lane 5), or B (lane 6) alone could effectively compete away Mad binding, while only the unlabeled full silencer element (lane 8) or site A (lane 9) could effectively compete for Med binding but site B (lane 10) could do to less extent; and,

FIG. 15E is a current model for how BMP niche signals control GSC identity by directly repressing bam transcription; BMP signals from cap cells produce the hihghest levels of pMad, which associates with Med and translocates into the nucleus in GSCs; the Mad and Med-containing protein complex directly occupies the bam silencer to repress its transcription in GSCs; as a cystoblast moves away from cap cells and proceeds to differentiate in a progressive manner, levels of pMad are gradually reduced to the critical threshold level when Bam transcription is derepressed and activated in more mature cystoblasts by an unknown activator.

### DETAILED DESCRIPTION OF THE INVENTION

Stem cells must remain undifferentiated and continue self-renewal at every cell division. In order for a stem cell to maintain its identity, it at least needs to repress the genes that are important for differentiation of stem cell daughters. Toward that end, a direct connection has been discovered between niche signals and repression of differentiation genes in stem cells. The present discovery, therefore, generally provides methods and compositions that may be employed to control stem cell self-renewal and differentiation. More particularly, the current invention is directed toward the elucidation of a signal transduction pathway that controls the expression of a gene necessary for differentiation of stem daughter cells. By modulating the levels of key regulatory molecules within this pathway, the fate of stem cell self-renewal and differentiation may be controlled. In addition, the present invention also provides mutant organisms, tissues, cells, as well as pathways where the level of key molecules within the pathway is modulated.

BMP Signaling Pathways in Stem Cells

One aspect of the current invention, therefore, encompasses the use of Drosophila germline stem cells, including cells derived from either the ovary or testis, as a model to elucidate key regulatory molecules in a BMP signaling pathway controlling stem cell fate. By examining this model, it has been discovered that one mechanism by which niche signals, such as BMP signaling, control stem cell fate is via directly repressing expression of a differentiation gene. A comparison of components of the BMP signaling pathway, upstream regulators, and downstream targets show them to be highly conserved across a number of diverse species (Bitgood and McMahon, 1995; Padgett et al., 1998). For example, such conservation has been observed in mammals, amphibians, birds, fishes, invertebrates like worms (e.g., Helminthes, Nematodes) and insects (e.g., Anopheles, Drosophila). Thus, the present invention should not be limited in its usefulness to Drosophila melanogaster.

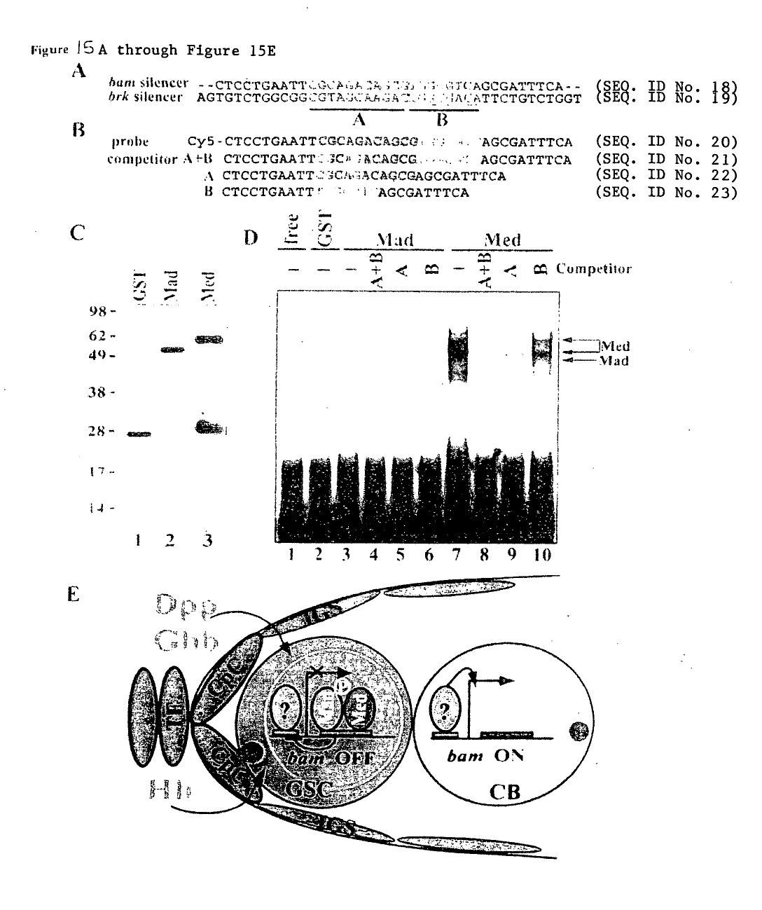

Generally speaking, and by way of a non limiting example, an overview of the BMP signaling in Drosophila germline stem cells is illustrated in FIG. 15E. Referring to FIG. 15E, BMP polypeptides from the germline stem niche cell activate their signaling cascade in germline stem cells, which leads to Mad phosphorylation in the stem cells. Mad phosphorylation then results in the translocation of the Mad and Med complex into the nucleus, which directly binds to the bam promoter and represses the transcription of bam. A more detailed explanation of this pathway, with references to results depicted in the examples, is described below. As non limiting examples, a detailed description of BMP signaling in Drosophila germline stem cells is provided below. In particular, slight differences in this signaling pathway between testis and ovary Drosophila stem cells is delineated.

BMP Signaling in Testis Germline Stem Cells

Examples 1-6 describe results showing molecular and genetic evidence that the BMP polypeptides, gbb and dpp, are both expressed in the testis and work cooperatively to maintain germline stem cells, at least in part, by preventing bam transcription in germline stem cells. In gbb mutant testis, for example, germline stem cells are lost very rapidly, but cystoblasts still develop into 16-cell cysts, illustrating that gbb functions specifically to control germline stem cell maintenance during germ cell development in the testis. Surprisingly, it was found that mutations in dpp have very little effect on germline stem cell maintenance. This is in contrast with the pivotal role of dpp in the ovary, as described below. But a mutation in one copy of the gbb gene greatly enhances the stem cell loss phenotype of dpp mutants, even though heterozygous gbb males have normal germline stem cell numbers, indicating that dpp and gbb work cooperatively to control germline stem cell maintenance.

Generally speaking in the Drosophila testis, therefore, dpp plays a less important role than gbb does with regards to germline stem cell regulation. In the Drosophila ovary, in contrast, both dpp and gbb play equally important roles in the regulation of germline stem cells. While dpp overexpression in the ovary completely blocks cystoblast differentiation, causing the accumulation of germline stem cell-like germ cells, overexpression of either dpp or gbb has little effect on differentiation of gonialblasts in the testis. These observations suggest that BMP signaling is essential for maintaining germline stem cells in both sexes, but that the gbb and dpp signals contribute differently.

It has also been discovered that gbb signals through previously defined dpp receptors to regulate the phosphorylation of Mad. dpp is known to require punt, tkv, and sax receptors for its signaling in patterning the embryo and imaginal discs (Brummel et In As detailed in the examples, pMad in gbb mutant germiline stem cells was severely reduced just like inpunt mutant germline stem cells. Moreover, it has been discovered that Dad, as it is with dpp, is also a gbb responsive gene in the Drosophila testis. Taken together these results show that a gbb signal through common dpp receptors to promote Mad protein phosphorylation and activates Dad transcription in germline stem cells just like dpp does.

Moreover, it has also been discovered that BMP signaling, mediated by dpp and gbb, is essential for maintaining germline stem cells in the testis. For example, it was shown that forced expression of bam in germline stem cells caused them to differentiate into gonialblasts, which suggested that bam is sufficient for germline stem cell differentiation. Normally, bam transcription is absent in germline stem cells, suggesting that an active mechanism exists to repress bam expression in germline stem cells. The mechanism shown herein is mediated by BMP signals that originated from the surrounding somatic niche cells. In the testis, the germline stem cells mutant for gbb, punt, and Med have elevated bam transcription. Moreover, dpp overexpression leads to bam transcriptional repression in all the germ cells of the testis.

BMP Signaling, in Ovary Germline Stem Cells

In the Drosophila ovary, as with the Drosophila testis, germline stem cells are located in a niche consisting of neighboring somatic cells. dpp has previously been shown as a factor that originates from the niche and is necessary for maintaining germline stem cells in the Drosophila ovary (Xie and Spradling, 1998, 2000). It has been discovered herein that gbb, in addition to dpp, functions as an essential niche signal for maintaining ovary stem cells. For example, loss of gbb function leads to germline stem cell differentiation and stem cell loss just like dpp mutations do. Moreover, similar to the Drosophila testis, gbb is expressed in the somatic cells, but not in germ cells, demonstrating that gbb is another niche signal that controls germline stem cell maintenance.

As stated above with respect to testis stem cells, it is believed that dpp and gbb function cooperatively to signal through common receptors such as punt, sax, and tkv to regulate various downstream events such as Mad phosphorylation. It has been discovered that both dpp and gbb function as short-range signals in the germline stem niche cell since their signaling activities, monitored by pMad and Dad expression, are restricted to germline stem cells and some cystoblasts. It has also been discovered that up-regulation of bam in germline stem cells is associated with stem cell loss in dpp and gbb mutants. Moreover, levels of pMad are related to bam transcriptional repression in both wild-type and BMP signaling-defective germaria. Taken together, the present discovery demonstrates that BMP signals maintain germline stem cells, at least in part, by repressing bam transcription in germline stem cells in the Drosophila ovary as well as in the Drosophila testis.

In addition, while not being bound to any particular theory, gbb also likely functions to augment the dpp signal in the regulation of germline stem cells in the Drosophila ovary. By way of example, in dpp and gbb mutants, pMad accumulation in germline stem cells is severely reduced, but not completely diminished. dpp overexpression results in complete suppression of cystoblast differentiation and complete repression of bam transcription in the germ cells, while gbb overexpression does not have obvious effects on cystoblast differentiation or bam transcription.

Recent promoter analysis shows that bam is transcriptionally repressed in GSCs through a defined silencer (Chen and McKearin, 2003). These observations support the model detailed herein that BMP signals from the niche maintain adjacent germ cells as GSCs by actively suppressing bam transcription and thus preventing differentiation in to cystoblasts. Furthermore, Med and Mad can bind to the defined bam silencer in vitro, which also supports that BMP signaling acts directly to repress bam transcription. While it remains unclear how the binding of Med and Mad to the bam silencer achieves bam transcriptional repression in GSCs, Mad and Med belong to the Smad protein family, which are known to function as transcriptional activators by recruiting co-activators with histone acetyltransferase activity (reviewed by Massague and Wotton, 2000). For example, in the wing disc, shn is proposed to function as a switch factor that converts the activating property of Mad and Med proteins into transcriptional repressors (Muller et al., 2003). But in the embryo, shn is also required for Mad and Med-mediated activation in addition to its requirement for brk repression with Mad and Med proteins (Torres-Vazquez et al., 2001). Therefore, shn itself may not function as a switch factor for determining activation or repression by Smads. Perhaps, the function that shn elicits with Smads depends on the presence of other co-repressors or co-activators in a given cell type. shn may recruit either a co-repressor or co-activator to the Smad complex through protein-protein interactions. Possibly, the Mad-Med complex could also recruit shn to the bam repressor element just as it does for the brk repressor in the wing disc (Muller et al., 2003). Consistent with the possible role of shn in repressing bam expression in GSCs is the observation that GSCs that lose shn function differentiate and thus are lost (Xie and Spradling, 2000).

It was also discovered that dpp and gbb function as short-range signals in the GSC niche in both testis and ovaries. As detailed in the examples, gbb is expressed in inner sheath cells and other somatic cells, possibly including cap cells. In both dpp and gbb mutant germaria, GSCs are lost rapidly but their differentiate progeny, cystoblasts, develop normally into 16-cell germline cysts, suggesting that the important role of BMP signaling in the germarium is to maintain GSCs. There are several models that can be proposed to explain how BMP signaling is involved in GSC maintenance. First, even though dpp and gbb mRNAs are broadly expressed in the somatic cells of the germarium, they are primarily translated in the neighborhood of cap cells and, therefore, their activities are concentrated around GSCs. Second, even if dpp and gbb proteins are broadly distributed, such specificity on GSCs can still be achieved through localized activation of these two proteins around cap cells. dpp is known to bind to Sog, and thus loses its ability to bind to its receptors after its association with Sog (Biehs et al., 1996; Marques et al., 1997). A metalloprotease, Tolloid (tld), can cleave Sog and release dpp, which is then free to bind to its receptor (Marques et al., 1997). Interestingly, tld is specifically expressed in cap cells. Finally, other signals from cap cells help BMP signals achieve specificity on GSCs by activating the expression of other genes in GSCs that help BMP signal transduction in GSCs. It was also discovered that BMP signaling appears to elicit different levels of responses in GSCs and cystoblasts. These experimental observations indicate that the cap cells are likely a source for active short-ranged BMP signals. Supporting this idea is the observation that when GSCs lose contact with the cap cells by removal of adherens junctions, they move away from the niche and then are lost (Song et al., 2002).

Modulating Participants of the BMP Signaling Pathway

One aspect of the invention provides a means to control stem cell fate by modulating the level of one or more regulatory molecules within a BMP signaling pathway. In particular, the invention provides stem cells, tissues and organisms where stem cell fate is controlled via modulation of a regulatory molecule within a BMP signaling pathway. For example, increasing BMP signaling results in an increase in the number of stem cells in a given population relative to wild type, by repression of bam transcription. In contrast, decreasing BMP signaling results in a decrease in the number of stem cells in a given population relative to wild type (i.e., increased differentiation) by facilitation of bam transcription. The level of one or more of the participants of BMP signaling may be modulated in accordance to achieve the desired result (i.e., increased or decreased differentiation) with methods known in the art or as described herein. As used herein, these participants are collectively referred to as a “participant” or “participants of BMP signaling.”

Generally speaking, BMP signaling involves a signal transduction pathway that is initiated when a BMP polypeptide binds to a receptor that typically results in the phosphorylation of serine/threonine residues. This receptor phosphorylation, concomitantly, results in a cascade reaction of components of the pathway (i.e., signal transducers such as, for example, transcription factors) that communicates the signal. For example, a signal may be communicated from a BMP polypeptide binding at the cell surface to the nucleus where gene expression of downstream targets are either activated or inhibited, such as detailed herein for bam repression. Thus, BMP signaling may be modulated at one or more steps in this pathway, or by affecting upstream regulators or downstream targets of this signaling pathway. Modulation (i.e., stimulation or repression) of BMP signaling may be accomplished directly on the stem cell or indirectly through other cells, such as through niche stem cells or in a mixed cell population (e.g., feeder layer).

In one embodiment, the participant of BMP signaling that is modulated is a smad protein. Non-limiting examples of smad proteins include mothers against dpp (mad), Medea (Med), and daughters against dpp (Dad) (Sekelsky et al., 1995; Tsuneizumi et al., 1997; Hudson et al., 1998; Wisotzkey et al., 1998; Das et al., 1998; Inoue et al., 1998). Smads are proteins that transduce signals on behalf of TGF-β family members, or inhibit TGF-β signal transduction. A paradigm for TGF-β signal transduction has been developed from several experimental systems. In Drosophila, as detailed herein, gbb and dpp bind both type I and II receptors to allow the constitutively active punt kinase to phosphorylate and activate type I kinases, which phosphorylate Mad. The phosphorylated Mad brings Med into the nucleus as a transcriptional activator to stimulate BMP targeted gene expression.

In yet another embodiment, the participant of BMP signaling that is modulated is one or more receptors on a stem cell that is responsive to BMP polypeptide signaling. In one alternative of this embodiment, the BMP receptor is a type I serine/threonine. By way of example, the BMP receptors saxophone (sax) and thick veins (tkv) encode type I serine/threonine kinase transmembrane receptors. In an alternative of this embodiment, the BMP receptor is a type II serine/threonine kinase transmembrane receptor. By way of example, punt is a type II receptor.

In yet another embodiment, the participant of BMP signaling that is modulated is a gene encoding a BMP polypeptide or a BMP polypeptide itself. By way of example, in Drosophila, suitable BMP polypeptides include dpp (SEQ ID No. 9), gbb (SEQ ID No. 10), and scw (SEQ ID No. 11). Suitable mammalian homologs of dpp, gbb, scw include BMP-2 (SEQ ID No. 12), BMP-4 (SEQ ID No. 13), BMP-5 (SEQ ID No. 14), BMP-6 (SEQ ID No. 15), BMP-7 (SEQ ID No. 16) and BMP-8 (SEQ ID No. 17), respectively. Other related members of the TGF-β family, their receptors, or other components of their signaling pathways, are also suitable for use in the present invention. See, e.g., U.S. Pat. Nos. 5,011,691, 5,013,649, 5166,058, 5,168,050, 5,216,126, 5,324,819, 5,354,557, 5,635,372, 5,639,638, 5650,276, and 5,854,207, all of which are hereby incorporated by reference.

In certain aspects, a polypeptide that is a homolog, ortholog, or degenerative variant of a BMP polypeptide is also suitable for use in the present invention. Typically, the subject polypeptides include fragments that share substantial sequence similarity, binding specificity and function with a BMP polypeptide, as defined herein. suitable homologs or degenerative variants preferably share at least 50% sequence homology with a BMP polypeptide, more preferably, 75%, and even more preferably, are greater than about 90% homologous in sequence to BMP polypeptide. Typically, sequence differences between a selected homolog or variant and a BMP polypeptide will include a number of conservative amino acid substitutions. A “conservative substitution” is a substitution that does not abolish the ability of a subject polypeptide to participate in BMP signaling, as described herein.

In determining whether a polypeptide is substantially homologous to a BMP polypeptide, sequence similarity may be determined by conventional algorithms, which typically allow introduction of a small number of gaps in order to achieve the best fit. In particular, “percent homology” of two polypeptides or two nucleic acid sequence sequences is determined using the algorithm of Karlin and Altschul (Proc. Natl. Acad. Sci. USA 87:2264-2268, 1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to a nucleic acid sequence molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul et al. (Nucleic acid sequences Res. 25:3389-3402, 1997). When utilizing BLAST and Gapped BLAST programs, the default parameters of the respective programs (e.g., XBLAST and NBLAST) are employed. See http://www.ncbi.nlm.nih.gov for more details.

It is also contemplated that BMP signaling is not confined to any one type of BMP polypeptide or any one type of BMP receptor because of the ability of evolutionarily diverged components of the BMP signal transduction pathway or different types of BMP polypeptides, BMP receptors, and smads to be functional equivalents of each other. For example, there appears to be crosstalk between dpp/Tkv signaling and gbb/Sax signaling and one signal transducer acts in different signaling pathways. By way of further example, a mixture of BMPs could be added to defined culture medium or be present in conditioned culture medium such that dpp and gbb would synergize in initiating BMP signaling through more than one different types of BMP receptor. As another example, one type of signal transducer could stimulate signaling through more than one different types of BMP receptor.

In order to modulate BMP signaling, therefore, the level of one or more participants in the pathway is altered relative to wild type. Enhancing BMP signaling activities can be achieved, for example, by reducing the presence of Dad proteins, such as human Smad6 and Smad7. Vertebrate Smad6 and Smad 7 interact with Type I receptors, and are known to inhibit both TGF-β and BMP signaling in cultured cells and frog embryos. Thus, disinhibition of TGF-β-family members by inhibiting certain Smads promotes BMP-like signaling cascades. Additionally, enhancing the function of BMP responsive receptors, such as Sax, Tkv, and Punt in Drosophila, and BMP receptors BMPR-II, ActR-II, Act-IIB, BMPR-IA, and ActR-I in humans may increase BMP signaling activities. Other downstream positive regulators of BMP signaling include Mad, Med, and Dad, proteins in Drosophila, and Smad1, Smad4 and Smad5 in humans.

In a further embodiment, one method for the enhancement of BMP signaling may be facilitated by removal of the dpp and gbb inhibitor Dad or other smad protein activity from the germline stem cells. Dad is induced by dpp or gbb signaling, but then acts to downregulate the very pathway that activated its production. This method could also be practiced with other negative regulators of BMP signaling and, in particular, inhibitory smads. In contrast, brinker (brk) is a target gene repressed by BMP signaling and, because it is itself a transcriptional repressor, the net effect of repressing expression of the Brk repressor is to upregulate Brk-regulated target genes. This results in the increased production of Brk-regulated target genes following BMP signaling. Hence, BMP signaling can be stimulated or repressed by appropriate manipulation of Smads or target genes which are regulated by BMP signaling (i.e., increasing or decreasing their effects as appropriate to achieve stimulation or repression of BMP signaling). It is believed that the roles of Dad and Brk, like the rest of the pathway, are conserved in mammals.

In an additional embodiment, to stimulate BMP signaling, the expression of a positive signal transducer may be increased (e.g., more transcripts and/or translated products) or mutated to a gain-of-function phenotype to increase activity of the subject signal transducer. Alternatively, the expression of a negative signal transducer may be decreased (e.g., fewer transcripts and/or translated products) or mutated to a loss-of-function phenotype to decrease activity of that signal transducer. Additionally, a downstream target gene of BMP signaling could be directly activated or inhibited without BMP binding to its receptor by genetic engineering using.

In yet a further embodiment, the endogenous BMP activity in the cells or exogenous BMP activity outside of the cells, especially if ligand is the limiting component in BMP signaling, may be increased. For example, BMP expression may be increased in a stem cell and stimulate BMP signaling through an autocrine mechanism. Alternatively, BMP expression may be increased in a non-stem cell or a feeder cell, and then BMP activity could be secreted and taken up by the stem cell or brought into contact with the surface of the stem cell. In still an additional embodiment, BMP could also be added to the extracellular space or culture medium.

Methods for Controlling Stem Cell, Stem Cells, Stem Cell Populations and Germline Tissue

Another aspect of the invention provides methods to control stem cell fate and the stem cells, stem cell populations and tissues produced via these methods. In particular, stem cell fate is controlled by modulating at least one of the above-identified participants of BMP signaling. Depending upon the embodiment, properties of stem cells that may be controlled by the practice of the invention include the following: pluripotency, totipotency, committing to one or more differentiating cell lineages, giving rise to multiple different types of progenitors and/or differentiated cells, contributing to the germline and combinations thereof. Generally speaking, if BMP signaling is increased relative to wild-type levels, stem cells in a population may be expanded in total number or concentration relative to non-stem cells (i.e., an increase in abundance), extended in the time between a stem cell's birth and its death or apoptosis (i.e., an increase in lifetime), or combinations thereof. Alternatively, if BMP signaling is decreased or repressed relative to wild-type levels, stem cells or tumor cells in a population may be reduced in total number or concentration, or even eliminated.

### Maintaining the Undifferentiated State of a Stem Cell

One alternative of this embodiment encompasses a method to maintain the undifferentiated state of an isolated stem cell. Also included in this aspect of the invention are the stem cells, isolated stem cell populations and tissue produced via the described methods.

Typically, an isolated stem cell or an isolated population of stem cells is contacted with an isolated stem niche cell or a population of stem niche cells. While the two cell types may be contacted either in vivo or in vitro, preferably they are contacted in vitro, such as in culture. Upon contact of the stem cell with the stem niche cell, a molecule expressed from the stem niche cell activates a signal transduction cascade in the stem cell such that the signal transduction cascade directly causes repression of a gene in the stem cell necessary for differentiation of a stem daughter cell.

The molecule expressed from the stem niche cell in this embodiment generally may be a number of different molecules to the extent that the molecule alters signal transduction in the stem cell so as to cause repression of a gene necessary for stem cell differentiation. For example, the molecule may be an organic compound, a nucleic acid sequence, a peptide or a protein. In one preferred embodiment, however, the signal transduction cascade that is activated is a BMP signal transduction cascade that comprises at least one molecule selected from the group consisting of tkv, sax, punt, pMad, Mad, Med, Dad and shn; the repressed gene is bar; and the molecule is any of the participants of BMP signaling identified above. In one preferred alternative of this embodiment, the participant of BMP signaling is a BMP polypeptide. Examples of Drosophila BMP polypeptides include dpp, gbb, and scw. Suitable mammalian homologs of dpp, gbb, and scw include BMP-2/4, BMP-5/8, BMP-2, BMP-4, BMP-5, BMP-6, BMP-7 and BMP-8. In a particularly preferred embodiment, the BMP polypeptide is gbb. In another particularly preferred embodiment, the BMP polypeptide encompasses a combination of gbb and dpp.

Stem cells utilized in the method of the invention may be from a variety of suitable sources and will typically have receptors for BMP polypeptides, especially gbb or a homolog, or will at least be responsive to BMP signaling. Cell populations may be derived from the germline or somatic (or mixed), male or female, dividing or quiescent, vertebrate or invertebrate, present in situ or isolated, partially or substantially purified, and combinations thereof. In one preferred embodiment, germline stem cells and in particular, Drosophila germline stem cells are employed. Germline stem cells and surrounding cells may be from either an adult (e.g., ovary, and testis) or an embryo. When the germline stem cells are from Drosophila, they may be suitably isolated from the testis, ovary, especially the apical tips of either the testes or ovariole. While the stem cells may be provided in situ as part of an intact organism or they may be cultured in vitro, preferably they will be cultured in vitro. For in vitro culturing, cells may be obtained directly from an organism (i.e., primary culture) or they may be passaged through several cultures (e.g., at least five, ten, or twenty times) to expand their number (e.g., at least two, ten, or 100 times more than the original number). Irrespective of the embodiment, the stem cells may be isolated by methods generally known in the art.

The stem niche cells employed in the invention are generally somatic cells, although the stem niche cells may also encompass some germline cells. For example, a “niche” is generally defined by surrounding somatic cells or a feeder layer comprised of somatic cells may provide cell contact and other extracellular signals to maintain and/or propagate stem cells. Typically, the somatic cells comprising a niche include terminal filament cells, cap cells, and inner sheath cells from the ovary and hub cells from the testis. Preferably, stem niche cell populations employed include cells expressing one or more BMPs; more preferably, BMP is secreted by non-stem cells and binds to receptors of stem cells to stimulate BMP signaling. In addition, a feeder layer may be provided that provides certain essential extracellular signals by, for example, genetically manipulating cultured cells to express and secrete a BMP that then binds to its receptor on the stem cells. The stem niche cells may be isolated by any method generally known in the art.

Stem cells produced according to the practice of the present invention may be totipotent or pluripotent, male or female, germline or somatic, dividing or quiescent, vertebrate or invertebrate, present in situ or isolated, partially or substantially purified of differentiate cells, and combinations thereof. Typically, proliferating stem cells are diploid, entering meiosis and the later stages of gametogenesis is part of the program of differentiation for male or female germline stem cells. By differentiating, stem cells may differentiate into cells of the hematopoietic, immune, or nervous systems or the like. Preferably, stem cells that are germline stem cells maintained and/or propagated by the present invention retain the potential to later differentiate and thereby contribute to oogenesis or spermatogenesis, all three germ layers (i.e., endoderm, mesoderm, ectoderm), multiple differentiated cell lineages, and combinations thereof.

Yet another aspect of the invention provides a method to maintain the undifferentiated state of an isolated Drosophila germline stem cell. Generally, germline stem cells and surrounding cells may be isolated from either an adult (e.g., ovary, and testis) or an embryo by any method generally known in the art, such as the isolation methods described herein. Suitable germline stem cells, for example, may be isolated from the testis, ovary, or an embryo especially the apical tips of either the testes or ovariole. Typically, the germline stem cells will be substantially pure, but they may include other cell types such as somatic cells. In one embodiment, the germline stem cells are isolated from an ovary. In a preferred embodiment, the germline stem cells are isolated from the testes. The isolated germline stem cells are then contacted with an isolated gbb polypeptide. Upon contact of the isolated germline stem cells with the gbb polypeptide, the gbb polypeptide activates a signal transduction cascade in the germline stem cells such that the signal transduction cascade directly causes repression of bam in the germline stem cell. Typically, the signal transduction cascade will include at least one molecule selected from the group consisting of tkv, sax, punt, pMad, Mad, Med, Dad, and shn. The gbb polypeptide may be obtained from a number of suitable sources. In one embodiment, the gbb polypeptide will be produced from an isolated stem niche cell selected from the group consisting of a terminal filament cell, a cap cell, an inner sheath cell, a hub cell, and a progenitor cell. In another embodiment, the gbb polypeptide will be produced by recombinant means such as from a vector comprising a nucleic acid sequence encoding a gbb polypeptide. In yet another embodiment, the gbb polypeptide will be a produced by synthetic means such as by direct synthesis. While the isolated germline stem cells and the gbb polypeptide may be contacted either in vivo or in vitro, preferably they are contacted in vitro, such as in culture. In alternative embodiments, the isolated germline stem cells may be contacted with other participants of BMP signaling in addition to being contacted with a gbb polypeptide. In a preferred embodiment, for example, the isolated germline stem cells are also contacted with another BMP polypeptide such as dpp.

In still another embodiment of the invention, stem cells are maintained in an undifferentiated state by increasing the expression of a nucleotide encoding a participant in BMP signaling identified above including, tkv, sax, punt, Mad, Med, and Dad. In a preferred embodiment, expression of a nucleotide encoding a BMP polypeptide is increased within the stem cell population. In a particularly preferred embodiment, the BMP polypeptide is gbb. In an alternative embodiment, the BMP polypeptide is dpp or a combination of gbb and dpp. The level of BMP polypeptide may be increased in the germline stem cell population by methods generally known in the art. In one embodiment, the BMP polypeptide will be overexpressed in the stem niche cells by methods known in the art. In one such method, an appropriate vector having a nucleic acid sequence encoding a BMP polypeptide, such as gbb, is introduced into the germline stem cell in accordance with methods known in the art, such as by mechanical technologies. An “appropriate vector” is typically a vector that contains the necessary elements for transcriptional and translational control of the inserted coding sequence in a suitable host. These elements generally will include regulatory sequences, such as enhancers, constitutive and inducible promoters, and 5' and 3' untranslated regions in the vector and in polynucleotide sequences encoding a target polypeptide. Such elements may vary in their strength and specificity. Specific initiation signals may also be used to achieve more efficient translation of nucleotide sequences encoding a target polypeptide. These signals, for example, include the ATG initiation codon and adjacent sequences (e.g. the Kozak sequence). In cases where nucleotide sequences encoding the subject polypeptide and its initiation codon and upstream regulatory sequences are inserted into the appropriate expression vector, no additional transcriptional or translational control signals may be needed. But in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (See, e.g., Scharf, D. et al. (1994) Results Probl. Cell Differ. 20:125-162).

Depending upon the embodiment, either eukaryotic or prokaryotic vectors may be used. Suitable eukaryotic vectors that may be used include MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3′SS, pBlueBacIII, pCDM8, pcDNA1, pZeoSV, pcDNA3, pREP4, pCEP4, and pEBVHis vectors. The MSCV or Harvey murine sarcoma virus is preferred. Suitable prokaryotic vectors that can be used in the present invention include pET, pET28, pcDNA3.1/V5-His-TOPO, pCS2+, pcDNA II, pSL301, pSE280, pSE380, pSE420, pTrcHis, pRSET, pGEMEX-1, pGEMEX-2, pTrc99A, pKK223-3, pGEX, pEZZ18, pRIT2T, pMC1871, pKK233-2, pKK38801, and pProEx-HT vectors.

Methods that are well known to those skilled in the art may be used to construct expression vectors containing a desired sequence and appropriate transcriptional and translational control elements. These methods include, for example, in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16).

Depending upon the embodiment, either a conditional recombination sequence or mutant sequence may be inserted into a vector. The vector for forming the conditional mutant will include the targeted BMP nucleic acid sequence, preferably flanked by recombination sites for the conditional sequence. The conditional vector is structured such that the targeted, recombination-site flanked gene or nucleotide sequence will be cut from the genome to form a knockout mutant.

Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Rcipon (1998) Curr. Opin. Biotechnol. 9:445-450).

A further aspect of the invention comprises a method to repress the expression of bam in an isolated stem cell. As detailed above, repression of bar expression typically results in maintaining the undifferentiated state of a stem cell. The method involves contacting an isolated stem cell with an isolated BMP polypeptide. Upon contact with the stem cell, the BMP polypeptide activates a signal transduction cascade in the stem cell such that the signal transduction cascade directly causes repression of bam transcription in the stem cell. Typically, the signal transduction cascade will include at least one molecule selected from the group consisting of tkv, sax, punt, pMad, Mad, Med, Dad, and shn. Depending upon the embodiment, stem cell populations may be derived from the either germline or somatic (or mixed), male or female, dividing or quiescent, vertebrate or invertebrate, present in situ or isolated, partially or substantially purified, and combinations thereof. In one preferred embodiment, germline stem cells and in particular, Drosophila germline stem cells are employed. The BMP polypeptide may also be obtained from a number of suitable sources. Examples of Drosophila BMP polypeptides include dpp, gbb, and scw. Suitable mammalian homologs of dpp, gbb, and scw include BMP-2/4, BMP-5/8, BMP-2, BMP-4, BMP-5, BMP-6, BMP-7 and BMP-8. In a particularly preferred embodiment, the BMP polypeptide is gbb. In another particularly preferred embodiment, the BMP polypeptide encompasses a combination of gbb and dpp. In accordance with the practice of the invention, the BMP polypeptide may be produced synthetically, recombinantly or from an isolated stem niche cell selected from the group consisting of a terminal filament cell, a cap cell, an inner sheath cell, a hub cell, and a progenitor cell.

### Promoting Stem Cell Differentiation

A further aspect of the invention encompasses a method to promote the differentiation of at least one germline stem cell in Drosophila. In each of the embodiments detailed below, the germline stem cell may be either a testis stem cell or an ovary stem cell. In order to promote germline stem cell differentiation, bam is expressed and is preferably, overexpressed. Also included in this aspect of the invention are the stem cells, isolated stem cell populations and tissue produced via the described methods.

According to the method of the invention, a number of suitable means may be employed to promote bam expression or overexpression and thereby, promote germline stem cell differentiation. In one alternative embodiment, at least one nucleic acid sequence encoding any of the above-identified participants involved in BMP signaling is mutagenized such that the polypeptide, when expressed in the Drosophila stem niche or germline stem cell, is rendered non functional. The subject nucleic acid sequence may be mutagenized by methods known in the art such as by a frame shift mutation, deletion mutation, loss of function, point or substitution mutation. By way of example, the participant of BMP signaling may be selected from the group consisting of tkv, sax, punt, Mad, Med, and shn. Typically, when any of tkv, sax, or punt is mutagenized a loss of receptor function will preferably occur. As a result, the germline stem cell is non responsive to a BMP polypeptide signal, such as gbb or dpp, bam is expressed in the germline stem cell, and differentiation of the germline stem cell is promoted. Alternatively, when any of Mad, Med, or shn is mutagenized, formation of the Mad/Med complex is substantially inhibited. As a result, the Mad/Med complex does not bind to bam and differentiation of the germline stem cell is promoted.

In another alternative of this embodiment to promote germline stem cell differentiation, a BMP polypeptide is substantially inhibited from either contacting, associating with or binding to BMP responsive receptors on the germline stem cell. As a result, the BMP signaling cascade in the germline stem cell is substantially inhibited and bam is expressed in the germline stem cell. The BMP polypeptide in this embodiment is preferably gbb, but can include any of the BMP polypeptide described herein or otherwise known in the art, such as dpp. In one embodiment, the BMP polypeptide is substantially inhibited from either contacting, associating with or binding to a BMP responsive receptor selected from tkv, sax, or punt located on the germline stem cell. A number of suitable methods are known in the art to inhibit such contact, association or binding. In one embodiment, a BMP nucleic acid sequence may be mutagenized such that when the BMP polypeptide is expressed it no longer interacts with the BMP responsive receptor in manner that results in BMP signal transduction. The BMP nucleic acid sequence may be mutagenized by any method generally known in the art including by a frame shift mutation, deletion mutation, loss of function, point or substitution mutation. In another embodiment, both the BMP polypeptide and the BMP responsive receptor are mutagenized. The BMP polypeptide is typically gbb or a combination of gbb and dpp and the BMP responsive receptor is generally tkv, sax, or punt.

In another embodiment to promote germline stem cell differentiation, the BMP polypeptide may be substantially inhibited from either contacting, associating with or binding to the BMP responsive receptor via use of an antagonist. The antagonist may have binding specificity for either the BMP polypeptide or the BMP responsive receptor. Moreover, an antagonist specific for the BMP polypeptide and an antagonist specific for the BMP responsive receptor may be utilized. In either alternative, the antagonist is preferably an antibody specific for either the BMP polypeptide or the BMP responsive receptor.

Antibodies to a BMP polypeptide or BMP responsive receptor may be generated using methods that are well known in the art. Such antibodies may include, but are not limited to, polyclonal, monoclonal, chimeric, and single chain antibodies, Fab fragments, and fragments produced by a Fab expression library.

For the production of antibodies, various hosts including goats, rabbits, rats, mice, humans, and others may be immunized by injection with a subject polypeptide that has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to either a BMP polypeptide or a BMP responsive receptor have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of the subject polypeptide amino acids may be fused with those of another protein, such as KLH, and antibodies to the chimeric molecule may be produced.

Monoclonal antibodies to either a BMP polypeptide or a BMP responsive receptor may be prepared using a technique that provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-45). Alternatively, techniques described for the production of single chain antibodies may be adapted, using methods known in the art, to produce a subject polypeptide-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments that contain specific binding sites for either a BMP polypeptide or a BMP responsive receptor may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the subject polypeptide and its specific antibody. A two-site, monoclonal-based immunoassay utilizing monoclonal antibodies reactive to two non-interfering BMP polypeptide epitopes is generally used, but a competitive binding assay may also be employed.

A number of methods such as Scatchard analysis in conjunction with radioimmunoassay techniques may be used to assess the affinity of antibodies for the subject polypeptide. Affinity is expressed as an association constant, Kₐ, which is defined as the molar concentration of subject polypeptide-antibody complex divided by the molar concentrations of free antigen and free antibody under equilibrium conditions. The Kₐ is determined for a preparation of polyclonal antibodies, which are heterogeneous in their affinities for multiple BMP polypeptide epitopes, represents the average affinity, or avidity, of the antibodies for BMP polypeptides. The Kₐ is determined for a preparation of monoclonal antibodies, which are monospecific for a particular BMP polypeptide epitope, represents a true measure of affinity. High-affinity antibody preparations with Kₐ ranging from about 10⁹ to 10¹² L/mole are preferred for use in applications in which the BMP polypeptide-antibody complex must withstand rigorous manipulations. Low-affinity antibody preparations with Kₐ ranging from about 10⁶ to 10⁷ L/mole are preferred for use in applications that ultimately require dissociation of BMP polypeptides, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

In another embodiment to promote the differentiation of a germline stem cell, antisense technology may be employed to modify gene expression of a participant in BMP signal transduction. This can be achieved by designing complementary sequences or antisense molecules (DNA, RNA, or modified oligonucleotides) to the coding or regulatory regions of the gene encoding a participant in BMP signaling such as any of the participants identified above. For example, the participant may include a BMP polypeptide such as gbb or dpp, a BMP responsive receptor, Med, or Mad. Such technology is well known in the art, and antisense oligonucleotides or larger fragments can be designed from various locations along the coding or control regions of sequences encoding the subject polypeptide. In a typical embodiment, the antisense molecule will bind to the subject participant in BMP signal transduction and will substantially inhibit its function. As a result of this diminished function, bam is expressed and germline stem cell differentiation is promoted.

Antisense sequences can be delivered intracellularly in the form of an expression plasmid that, upon transcription, produces a sequence complementary to at least a portion of the cellular sequence encoding the target protein. (See, e.g., Slater, J. E. et al. (1998) J. Allergy Clin. Immunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, suora; Uckert, W. and W. Walther (1994) Pharmacol. Ther. 63(3):323-347.) Other gene delivery mechanisms include liposome-derived systems, artificial viral envelopes, and other systems known in the art (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic acid sequences Res. 25(14):2730-2736.)

In a further embodiment to promote germline stem cell differentiation, bam may be overexpressed by a variety of suitable methods generally known in the art. In one such method, a vector encoding bam is introduced into the germline stem cell in accordance with methods known in the art, and as described herein. Depending upon the embodiment, either a conditional recombination sequence or mutant sequence may be inserted into a vector. The vector for forming the conditional mutant will include the targeted bam nucleic acid sequence, preferably flanked by recombination sites for the conditional sequence. The conditional vector is structured such that the targeted, recombination-site flanked gene or nucleotide sequence will be cut from the genome to form a knockout mutant.

Formation of Mutant Organisms

Another aspect of the invention encompasses the formation of a mutant non human organism. In one embodiment, a conditional BMP polypeptide mutant stem niche cell is formed by transfecting embryonic stem niche cells, with the BMP gene later rendered nonfunctional upon activation in a postnatal organism. In a particularly preferred embodiment, the BMP polypeptide is gbb. The conditional gene mutation in a pre-recombination organism is maintained dormant throughout gestation. The conditional gbb mutant stem niche cells can be formed in vivo, such as in a Mx1-Cre organism. Alternatively, Wt stem niche cells can be isolated and treated in vitro to obtain gbb mutant stem niche cell. A vector can be utilized to create a gbb gene recombination-induced conditional mutation in stem niche cells. In yet another embodiment, a gbb gene mutation can be directly induced in stem niche cells by a mutagen. The conditional knockout cells and organisms include pre-recombination and post-recombination cells and organisms.

Any of the eukaryotic vectors detailed herein can be used to transfect eukaryotic host cells including mammalian, amphibian, or insect cells, eukaryotic cells include human, mouse, and frog cells. The preferred process includes transfecting an embryonic stem cell of a selected species with the vector. The transfected embryonic stem cell is then transplanted into an adopted host mother. The embryonic stem cell will gestate to an embryo followed by birth of a conditional mutant organism. Thus, mutant offspring are formed, such as a gbb mutant organism. Specific conditionally active mutants include stem niche cells.

Typically, two organism lines (mouse, for example) are required for formation of a conditional gene deletion organism: a conventional transgenic line with, for example, Cre-targeted to a specific tissue or cell type, and a strain that embodies a target gene (endogenous gene or transgene) flanked by two recombination (LoxP, for example) sites in a direct orientation (“floxed gene”). When the target gene is the gbb gene, recombination occurs by excision and, consequently, inactivation of the floxed gbb target gene. Since recombination and gbb gene excision occurs only in those cells expressing Cre recombinase, the gbb target gene remains active in all cells and tissues that do not express Cre recombinase. A recombination activator, such as PolyI:C or interferon, which in turn triggers Cre recombinase expression, induces Gene excision. The recombination activator is preferably injected postnatally to ensure organism survival. Most preferably the recombination activator is injected at 0, 1, 2, or 20 days after birth, or anytime thereafter. Cre and FLP recombinase are exemplary recombinases that may be used. Cre recombinase is used to cleave Lox sites flanking the gbb gene. Alternatively, FLP recombinase can be used with FRT recombination sites flanking the gbb gene.

A number of suitable operative recombination activators may be utilized in the current invention. For example, operative recombination activators can include PolyI:C, interferon, or other interferon inducers. PolyI:C is a preferred recombination activator. The recombination activator induces Cre recombinase expression, which in turn results in excision of the Lox-flanked gbb nucleic acid sequence in cells of the mutant gbb organism.

In the germline tissue of the transfected animal, the resultant mutant gbb stem niche cell contains a conditional mutant gbb gene that can encode an inactive gbb polypeptide. Alternatively, the cells can be mutagenized and nonconditional. The gbb mutant stem niche cell can be a resting, self-renewing, proliferating, or differentiating cell. The gbb mutant stem niche cell may be made in vivo or in vitro by methods such as knockout organism formation, vector transfection, micro-vessel transfer, biolistic particle delivery, liposome-mediated transfer, electroporation, or microinjection of the gbb mutant gene.

In a particularly preferred embodiment, the mutant organism is a Drosophila fly. In one such embodiment, the mutant fly comprises a clonal population of testicular niche stem cells wherein gbb is knocked out. A mutant fly according to this embodiment typically has a decreased population of testicular stem cells compared to a wild type fly.

In yet another preferred embodiment, a mutant fly comprises a clonal population of testicular niche stem cells wherein gbb is overexpressed. A mutant fly according to this embodiment generally has an increase in the population of testicular stem cells compared to a wild type fly.

In still another embodiment, a mutant fly comprises a clonal population of testicular stem cells that have been mutagenized such that the cells are non responsive to a gbb polypeptide, thereby resulting in bam expression. A fly according to this embodiment will have a decreased population of testicular stem cells compared to a wild type fly.

Germline Stem Cell Cultivation Systems

Yet another aspect of the invention encompasses a germline stem cell cultivation system. For example, an in vitro germline stem cell cultivation system may be developed, wherein an activated germline stem cell population proliferates. The cultivation system includes an isolated germline tissue comprising germline stem cells, a culture medium, and an isolated stem cell activator. The activator operatively attaches to at least one stem cell in the population. Suitable activators include a mutant BMP receptor polypeptide, a mutant BMP receptor nucleic acid sequence, anti-BMP receptor antibody, anti-BMP antibody, or a Wt BMP antisense sequence. The germline tissue can be of mammalian origin, but is typically from a Drosophila. In particular, germline tissue can be isolated, containing cells that are then mutagenized to prevent a BMP polypeptide from interacting with a BMP receptor. Inhibition of this interaction, results in promotion of germline stem cell differentiation. In a particularly preferred embodiment, the BMP polypeptide is gbb or a combination of gbb and dpp.

An exemplary in vitro germline tissue cultivation system promotes germline stem cell differentiation in response to decreased BMP signaling. Other activators, such antagonists specific to participants in BMP signaling may be employed, including anti-BMP receptor antibodies or anti-BMP polypeptide antibodies. The cultivation system typically contains isolated germline tissue, culture medium, and an effective amount of a suitable activator, such as those described herein. Germline tissue from Drosophila may be used. Alternatively, instead of tissue, the cultivation system can contain an isolated germline stem cell population comprising at least 10⁴ cells. The germline stem cell population can be isolated by FACS methods using antibodies directed against germline stem cell-associated antigens, such as anti-BMP receptor polypeptide.

The activator can be placed in operative contact with the germline stem cell population by means of an activator insertion device. Activator insertion devices can be injection, diffusion, particle-mediated, micro-vessel encapsulation, or liposome encapsulation devices. An in vitro mutant BMP receptor germline stem cell cultivation system results, wherein a mutant germline stem cell population is promoted to differentiate, having the following: an isolated mutant gbb germline stem cell population comprising an inactive BMP receptor and culture medium. BMP receptor gene mutations introduced into the mutant germline stem cell can be frame shift, substitution, loss of function, or deletion mutations.

In an alternative in vitro cultivation system, germline tissue containing mutant BMP receptor stem cells is isolated and cultivated with culture medium in a culture vessel. The isolated mutant BMP receptor germline stem cells have inactive BMP receptor nucleic acid sequences and polypeptides.

Alternatively, an exemplary in vitro germline tissue cultivation system maintains the undifferentiated state of a germline stem cell in response to increased BMP signal. The germline tissue may be obtained and isolated from any of the sources identified above or as otherwise known in the art. In one preferred embodiment, the germline tissue comprises stem niche cells that have been mutagenized to overexpress a BMP polypeptide. In a particularly preferred embodiment, the BMP polypeptide is gbb or a combination of gbb and dpp. By way of non limiting example, a vector having a nucleotide sequence encoding a BMP polypeptide can be introduced into the stem niche cells causing the niche cells to overexpress the BMP polypeptide. Methods for introducing such a vector into stem niche cells are known in the art and are described in detail above.

Kits

A variety of kits can be formed either from the mutant or Wt polypeptides or the nucleic acid sequence sequences associated with germline tissue or stem cells described herein. Kits are described for detection of mutant or variant forms of the aforementioned nucleic acid sequence molecules, detection of expressed polypeptides or proteins, and measurement of corresponding levels of protein expression. Kits can detect the presence or absence of mutants or Wt forms of the nucleic acid sequence molecules, and their expressed amino acid sequences or polypeptide molecules. The kit will preferably have a container and either a nucleic acid sequence molecule or a polypeptide molecule, which includes any of the aforementioned sequences.

A kit of the present invention may include a container and any of the above described participants in BMP signaling. The kit typically will detect either a mutant or Wt polypeptide or nucleic acid sequence molecule participant of BMP signaling in either germline tissue or germline stem cells. For example, the kit may be suitably used to detect the presence of a mutant BMP receptor, gene, or polypeptide. The kit will also detect a mutant HFSC containing an inactive Bmpr1a receptor or gene. Kits to detect and quantify the presence in germline stem cells of BMP signaling markers such as BPM receptors including tkv, sax and punt; BMP polypeptides such as gbb, dpp, BMP-2/4, BMP-5/8, BMP-2, BMP-4, BMP-5, BMP-6, BMP-7 and BMP-8; Med; Mad; pMad; Dad; and shn or well as nucleic acid sequence markers are also contemplated to be within the present invention. These kits can be used for detection and quantitation of markers associated with germline stem cell proliferation and differentiation.

In summary, hybridization methodology and kits for the detection, identification, and quantitation of BMP signaling-associated nucleic acid sequence sequences in cells are set forth herein. Using these methods, participants of BMP signaling and mutant nucleic acid sequence sequences can be identified, characterized, and quantified. In addition, kits may be produced utilizing BMP signaling-derived nucleic acid sequence molecule standards, antibodies, and kit components as previously described.

All publications, patents, patent applications, and other references cited in this application are herein incorporated by reference in their entirety as if each individual publication, patent, patent application or other reference were specifically and individually indicated to be incorporated by reference.

### DEFINITIONS

The following definitions define terms used herein:

Activated mutant is a post-recombination organism, tissue, or cell wherein the mutant is obtained by injection of a recombination activator into a conditional mutant organism, tissue, or cell to induce a mutation event that results in inactivation of the targeted gene. For example, an activated BMP polypeptide mutant organism is a post-excision organism that resulted from PolyI:C injection of a conditional BMP polypeptide mutant organism to yield a nonfunctional BMP gene.

Activator is a molecule that activates a cellular activity. Cellular activities induced by the activator may be proliferation, self-renewal, differentiation, tumorigenesis, or apoptosis. A germline stem cell activator is one that generally activates proliferation, self-renewal, or differentiation. Activator can also refer to a molecule that induces recombination in a cell, such as those utilized in the Lox and Flp recombinase genetic systems. Examples of recombination activators are PolyI:C and interferon, which induce recombination in cells containing Lox or Frt flanked genes, generally resulting in inactivation of the target gene.

An amino acid (aminocarboxylic acid) is a component of proteins and peptides. All amino acids contain a central carbon atom to which an amino group, a carboxyl group, and a hydrogen atom are attached. Joining together of amino acids forms polypeptides. Polypeptides are molecules containing up to 1000 amino acids. Proteins are polypeptide polymers containing 50 or more amino acids.

Antibody (Ab) is any molecule that can bind specifically to an Antigen (Ag). Each Ab molecule has a unique Ag binding site that enables it to bind specifically to its corresponding antigen. Abs includes, but is not limited to, immunoglobulins of the IgG, IgA, IgM, IgD, and IgE classes. Abs are often produced by B cells and plasma cells in response to infection or immunization, bind to and neutralize pathogenic micro-organisms, or prepare microbes for uptake and destruction by phagocytes. Abs may also be produced in vitro by cultivation of plasma cells or B cells, or by utilization of genetic engineering technologies.

An antigen (Ag) is any molecule that can bind specifically to an antibody (Ab). Ags can stimulate the formation of Abs. Each Ab molecule has a unique Ag binding pocket that enables it to bind specifically to its corresponding antigen. Abs may be used in conjunction with labels (e.g., enzyme, fluorescence, radioactive) in histological analysis of the presence and distribution of marker Ags. Abs may also be used to purify or separate cell populations bearing marker Ags through methods, including fluorescence activated cell sorter (FACS) technologies. Abs that binds to cell surface receptor Ags can inhibit receptor-specific binding to other molecules to influence cellular function. Abs are often produced in vivo by B cells and plasma cells in response to infection or immunization, bind to and neutralize pathogens, or prepare them for uptake and destruction by phagocytes. Abs may also be produced in vitro by cultivation of plasma cells, B cells or by utilization of genetic engineering technologies.

Bone morphogenic proteins (BMPs) constitute a novel subfamily of the transforming growth factor type beta (TGF-beta) supergene family and play a critical role in modulating mesenchymal differentiation and inducing the processes of cartilage and bone formation. BMPs induce ectopic bone formation and support development of the viscera. Exemplary BMPs include those listed by the NcBI, such as human BMP-3 (osteogenic) precursor (NP001192), mouse BMP-6 (NP031582), mouse BMP-4 (149541), mouse BMP-2 precursor (1345611), human BMP-5 preprotein (NP 066551.1), mouse BMP-6 precursor (1705488), human BMP-6 (NP 001709), mouse BMP-2A (A34201), mouse BMP-4 (461633), and human BMP-7 precursor (4502427). The BMPs consist of at least eight members, BMP-2 through BMP-8A and BMP-8B. In embryogenesis, BMPs play roles in dorsoventral and/or anterior-posterior axis formation. Bone morphogenic protein (BMP) initiates, promotes, and regulates bone development, growth, remodeling, and repair. BMPs belong to the TGF-beta superfamily of structurally related signaling proteins. Members of this TGF-beta superfamily are widely represented throughout the animal kingdom and have been implicated in a variety of developmental processes. Proteins of the superfamily are disulfide-linked dimers composed of two 12-15 kDa polypeptide chains.

Chimera is an individual composed of a mixture of genetically different cells. By definition, genetically different cells of chimeras are derived from genetically different zygotes.

Conditional mutant is a pre-recombination organism, tissue, or cell wherein injection of a recombination activator into the conditional mutant organism, tissue, or cell induces a mutation event that results in inactivation of the targeted gene, resulting in formation of an activated target gene mutant organism.

Deletion mutations may be conditional knockout deletion mutations or conventional deletion mutations. Knockout deletion mutations are induced by administration of a recombination activator, such as PolyI:C, to a pre-excision mutant organism. Injection of the recombination activator results in excision or “knockout” of a portion of the genetic sequence from the nucleic acid sequence, thereby inducing a deletion mutation in the gene. Conventional deletion mutations may be single or multiple nucleotide deletions in a gene or chromosome.

Differentiation occurs when a cell transforms itself into another form.

Expression cassette (or DNA cassette) is a deoxyribonucleic acid (DNA) sequence that can be inserted into a cell's DNA sequence. The cell in which the expression cassette is inserted can be a prokaryotic or eukaryotic cell. The prokaryotic cell may be a bacterial cell. The expression cassette may include one or more markers, such as Neo and/or LacZ. The cassette may contain stop codons. In particular, a Neo-LacZ cassette is an expression cassette that can be placed in a bacterial artificial chromosome (BAC) for insertion into a cell's DNA sequence. Such expression cassettes can be used in homologous recombination to insert specific DNA sequences into targeted areas in known genes.

A gene is a hereditary unit that has one or more specific effects upon the phenotype of the organism; and the gene can mutate to various allelic forms. The gene is generally comprised of deoxyribonucleic acid or ribonucleic acid sequences (i.e., DNA, RNA).

A host cell is a cell that receives a foreign biological molecule, including a genetic construct or antibody, such as a vector containing a gene.

A host organism is an organism that receives a foreign biological molecule, including a genetic construct or antibody, such as a vector containing a gene.

Host cell is a cell that receives a foreign biological molecule, including a genetic construct or antibody, such as a vector containing a gene.

Host organism is an organism that receives a foreign biological molecule, including a genetic construct or antibody, such as a vector containing a gene.

Insertion device is a device that places an activator or oligonucleotide molecule in operative contact with a cell. Insertion devices can be activator insertion devices or oligonucleotide insertion devices. The activator or oligonucleotide molecule may be inserted within the cell or placed in contact with the cell surface. The insertion device can be injection, electroporation, transfection, vector, particle encapsulation, or liposome encapsulation devices.

Knockout is an informal term coined for the generation of a mutant organism (generally a mouse) containing a null or inactive allele of a gene under study. Usually the animal is genetically engineered with specified wild-type alleles replaced with mutated ones. Knockout also refers to the mutant organism or animal. The knockout process may involve administration of a recombination activator that excises a gene, or portion thereof, to inactivate or “knock out” the gene. The knockout organism containing the excised gene produces a nonfunctional polypeptide.

Label is a molecule that is used to detect or quantitate a marker associated with a cell or cell type. Labels may be nonisotopic or isotopic. Representative, nonlimiting nonisotopic labels may be fluorescent, enzymatic, luminescent, chemiluminescent, or colorimetric. Exemplary isotopic labels may be H³, C¹⁴, or P³². Enzyme labels may be horseradish peroxidase, alkaline phosphatase, or β-galactosidase labels conjugated to anti-marker antibodies. Such enzyme-antibody labels may be used to visualize markers associated with cells in hair follicle or other tissue.

Marker is an indicator that characterizes either a cell type or a cell that exists in a particular state or stage. A stem cell marker is a marker that characterizes a specific cell molecule that can be located a variety of places including within cells, on the surface of cells, or otherwise associated with cells.

Mutation is defined as a genotypic or phenotypic variant resulting from a changed or new gene in comparison with the Wt gene. The genotypic mutation may be a frame shift, substitution, loss of function, or deletion mutation, which distinguishes the mutant gene sequence from the Wt gene sequence.

Mutant is an organism bearing a mutant gene that expresses itself in the phenotype of the organism. Mutants may possess either a gene mutation that is a change in a nucleic acid sequence in comparison to Wt, or a gene mutation that results from the elimination or excision of a sequence. In addition polypeptides can be expressed from the mutants. Mutant may also refer to nucleic acid or polypeptide sequences themselves that result from gene mutation.

Nucleic acid or nucleotide sequence is a nucleotide polymer. Nucleic acid also refers to the monomeric units from which DNA or RNA polymers are constructed, wherein the unit consists of a purine or pyrimidine base, a pentose, and a phosphoric acid group.

Nucleotide sequence is a nucleotide polymer, including genes, gene fragments, oligonucleotides, polynucleotides, and other nucleic acid sequences.

Plasmids are double-stranded, closed DNA molecules ranging in size from 1 to 200 kilo-bases. Plasmids are capable of extrachromosomal replication, like other episomes; and some plasmids are capable of integrating into the host genome. Plasmids may be contained in cloning vectors for transfecting a host with a nucleic acid molecule.

PolyI:C is an interferon inducer consisting of a synthetic, mismatched double-stranded RNA. The polymer is made of one strand each of polyinosinic acid and polycytidylic acid. PolyI:C is 5′-Inosinic acid homopolymer complexed with 5′-cytidylic acid homopolymer (1:1). PolyI:C's pharmacological action includes antiviral activity.

Polypeptide is an amino acid polymer comprising at least two amino acids.

Post-excision mutant organism is an organism, a targeted gene, or sections thereof, wherein the targeted gene or section has been excised by recombination. The post-excision organism is called a “knockout” organism. Administration of a recombination activator, such as PolyI:C or interferon, can induce the recombination event resulting in target gene excision. A post-excision BMP polypeptide mutant organism is one in which the corresponding BMP gene has been inactivated.

Pre-excision mutant organism is one that has recombination sites flanking regions of target gene. The pre-excision organism generally has recombinase-encoded sites that can be induced to express Cre or Flp recombinase, but remain dormant or unexpressed until cells of the organism are exposed to a recombination activator. Administration of the activator to the pre-excision target gene-containing mutant organism under proper conditions can transform it into a post-excision gene-containing mutant organism.

Post-excision target gene-containing mutant organism is one which has been administered a recombination activator, such as PolyI:C, which excises a recombination site-flanked portion of the target gene, rendering it nonfunctional.

Proliferation occurs when a cell divides and results in progeny cells.

A selectable marker is a marker that is inserted in a nucleic acid sequence that permits the selection and/or identification of a target nucleic acid sequence or gene.

Self-renewal occurs where a cell reproduces an exact replicate of itself, such that the replicate is identical to the original stem cell.

Smad proteins are signal transducers that interact with BMP receptors. SMAD molecules are evolutionarily conserved proteins identified as mediators of transcriptional activation by members of the TGF-beta superfamily of cytokines, including TGF-beta, Activins, and BMP. Upon activation these proteins directly translocate to the nucleus where they may activate transcription (Datto et al.). Eight Smad proteins have been cloned (Smad 1-7 and Smad 9). Upon phosphorylation by the BMP Type I receptor, Smad1 can interact with either Smad4 or Smad6. The Smad1-Smad6 complex is inactive; however, the Smad1-Smad4 complex triggers the expression of BMP responsive genes. The ratio between Smad4 and Smad6 in the cell can modulate the strength of the signal transduced by BMP. Smad1,5,8 is also referred to as SMAD158. SMAD-1 is the human homologue of Drosophila Mad (Mad=Mothers against decapentaplegic). SMAD-1 has been shown to migrate into the nucleus in response to BMP-4. SMAD-5 sequences have been cloned. An analysis of various tumors demonstrates that mutations in various SMAD genes do not, in general, account for the widespread resistance to TGF-beta that is found in human tumors. SMAD-8 is a protein from Xenopus laevis distantly related to other SMAD proteins, and it modulates the activity of BMP-4.

A stem cell is defined as a pluripotent or multipotent cell that has the ability to divide (self-replicate) or differentiate for indefinite periods—often throughout the life of the organism. Stem cell self-renewal occurs when a stem cell divides to create an identical cloned replicate of itself. Under the right conditions, or given optimal regulatory signals, stem cells also can differentiate to transform themselves into the many different cell types that make up the organism. Multipotential or pluripotential stem cells possess the ability to differentiate into mature cells that have characteristic attributes and specialized functions, such as hair follicle cells, epidermis cells, intestinal cells, blood cells, cardiac cells, or nerve cells.

Stem cell marker is defined as a specialized protein or glycoprotein, associated with the stem cell, that is characteristic of that cell type. Stem cell markers often exist on the surface of cells, and these markers, sometime referred to as “receptors,” may have the functional capability of selectively binding or adhering to a “signaling” molecule antibodies. More particularly, the stem cell marker may characterize the stem cell as a multipotential or pluripotential cell type rather than as a differentiated cell type.

A stem cell population is a population that possesses at least one stem cell.

A vector is an autonomously self-replicating nucleic acid molecule that transfers a target nucleic acid sequence into a host cell. The vector's target nucleic acid sequence can be a Wt or mutant gene, or fragment derived therefrom. The vector can include a gene expression cassette, plasmid, episome, or fragment thereof. Gene expression cassettes are nucleic acid sequences with one or more targeted genes that can be injected or otherwise inserted into host cells for expression of the encoded polypeptides. Episomes and plasmids are circular, extrachromosomal nucleic acid molecules, distinct from the host cell genome, which are capable of autonomous replication. The vector may contain a promoter, marker or regulatory sequence that supports transcription and translation of the selected target gene. Viruses are vectors that utilize the host cell machinery for polypeptide expression and viral replication.

Wild type is the most frequently observed phenotype or genotype in a population, or the one arbitrarily designated as “normal.” Wild type is often represented by “+” or “Wt” symbols. The Wt phenotype is distinguishable from mutant phenotypic variations, and the Wt genotype is distinguishable from mutant genotypic variations.

As various changes could be made in the above compositions, methods, and products without departing from the scope of the invention, it is intended that all matter contained in the above description and in the examples given below, shall be interpreted as illustrative and not in a limiting sense.

### EXAMPLES

Role of BMP Niche Signaling in Drosophila Tests

Examples 1-6 detail how BMP signals from niche cells maintain germline stem cells by repressing bam transcription in the Drosophila testis. In examples 1-6, where indicated, the following experimental procedures and reagents were employed:

The Drosophila stocks used in the Examples were developed as follows: The following fly stocks used in the examples were described either in FlyBase or as otherwise specified: tkv⁸ and sax⁴ (Brummel et al., 1994); punt10460 and punt¹³⁵; Med²⁶ (Das et al., 1998); Dad-lacZ (Tsuneizumi et al., 1997); dpp^{hr4} and dpp^{hr56}; gbb⁴, gbb^{D4}, and gbb^{D20}; bam-GFP (Chen and McKearin, 2003); vasa-GFP (Nakamura et al., 2001); c587-gal4, hs-gal4 and nanos-gal4VP16 (Van Doran et al., 1998); UAS-dpp and UAS-gbb (Khalsa et al., 1998); hsFLP; FRT_{82B} armadillo-lacZ. Most stocks were cultured at room temperature. To maximize their mutant effects, dpp, gbb, and punt mutant adult females were cultured at 29° C. for 2-7 days.

Generating mutant GSC clones and overexpression—Clones of mutant GSCs were generated by Flp-mediated mitotic recombination, as described previously (Xie and Spradling, 1998). To generate the stocks for stem cell clonal analysis +FRT_{40A}/CyO, tkv⁸ FRT_{40A}/CyO and mad¹² FRT_{40A}/CyO males were mated with virgin females hs-FLP, armadillo-lacZ FRT_{40A}; FRT_{G13} sax⁴/CyO males were mated with virgin females hs-FLP FRT_{G13} armadillo-lacZ; FRT_{82B} Mea²⁶/TM3 Sb, FRT_{82B} punt¹⁰⁴⁶⁰/TM3 Sb, FRT_{82B} punt¹³⁵/TM3 Sb males were mated with virgin females hs-FLP; FRT_{82B} armadillo-lacZ.

To make mutant Med GSC clones for examining bam-GFP expression, bam-GFP/CyO; FRT_{82B} Med²⁶/TM3 Sb males were mated with virgin females hs-FLP; FRT_{82B} armadillo-lacZ. Two-day old adult non-CyO or non-Sb males carrying an armadillo-lacZ transgene in trans to the mutant-bearing chromosome were heat-shocked at 37° C. for three consecutive days with two one-hour heat-shock treatments daily separated by 8-12 hours. The males were transferred to fresh food every day at room temperature, and the testes were removed two days, one week, and two weeks after the last heat-shock treatment and then processed for antibody staining.

To construct the stocks for overexpressing dpp or gbb, nanos-gal4VP16 virgins were crossed with UAS-dpp and UAS-gbb males, respectively. The males that carried nanos-gal4VP16 and UAS-dpp or UAS-gbb were cultured at room temperature or at 29° C. for one week. For examining the expression of bam-GFP in the ovary overexpressing dpp or gbb, the bam-GFP/CyO; nanos-gal4VP16 virgins were used in the crosses.

The following protocol was utilized for measuring GSC loss in gbb mutants and marked GSCs, and examining bam-GFP expression in gbb, dpp, and punt mutant testes—To determine loss of marked mutant GSC clones, GSCs were marked in 1- to 2-day-old males of the appropriate genotype. Subsequently, testes were isolated from some of the males at 2-day, 1-week, and 2-week intervals and stained with anti-Hts and anti-LacZ antibodies. The percentage of testes containing one or more marked GSCs was determined by counts of 55 to 227 testes at each time point.

To measure stem cell loss in gbb mutant testes, the testes with different numbers of GSCs were determined based on anti-Hts and anti-Fas III antibody staining of gbb⁴/gbb^{D4} or gbb⁴/gbb^{D20} testes of different ages and different treatments. yw males carrying no gbb mutations served as a control. The 2-day old control and gbb mutant males were cultured at different temperatures, after they eclosed at 18° C. Values are expressed as the average GSC number per testis and/or the percentage of testes carrying no GSCs.

To examine bam-GFP expression in dpp, gbb, and punt mutant testes, males with the following genotypes were generated at 18° C.: bam-GFP gbb⁴/gbb^{D4}, bam-GFP gbb⁴/gbb^{D20}, bam-GFP dpp^{hr56}/dpp^{hr4} or punt10460/punt¹³⁵ bam-GFP, bam-GFP males carrying no mutations for gbb, dpp, or punt served as a control. All the control and mutant males were cultured at 29° C. for 4 days before their testes were isolated, stained with antibodies and compared for bam-GFP expression at identical conditions.

The TUNEL cell death assay was performed on the punt mutant testes following the ApopTag apoptosis detection kit manual (Intergen Company).

Immunohistochemistry—The following antisera were used to detect the described protein: polyclonal anti-Vasa antibody (1:2000) (Liang et al., 1994); monoclonal anti-Hts antibody (1:3); polyclonal anti-LacZ antibody (1:1000) (Cappel); polyclonal anti-GFP antibody (1:200) (Molecular Probes); polyclonal anti-pMad antibody (1:200) (Tanimoto et al., 2002). The immuno-staining protocol used in this study was described previously (Song et al., 2002). All micrographs were taken using a Leica SPII confocal microscope.

Detecting gene expression in purified component cells using RT-PCR—The tips of the testes for the males of the appropriate genotype were dissected and removed from the whole testes in Grace's media, and were dissociated with the collagenase II (Sigma) solution at a concentration of 6 mg/ml. After sorting of GFP-positive cells with the Cytomation MoFlo from the testes with GFP-marked hub cells, somatic cyst cells or germ cells, total RNAs were prepared using Trizol (Invitrogen) from the purified GFP-positive cells. This was done to amplify the RNA. The RNA samples were further amplified using the GeneChip Eukaryotic Small Sample Target Labeling Assay Version II (Affymetrix Inc.). After the RNA amplification, 1 ng or 10 ng or total RNAs was reverse-transcribed (RT) using SuperScriptIII First-Strand Synthesis System for RT-PCR according to manufacturers' protocol (Invitrogen). The following primers were used in this study:

dpp

(SEQ ID NO. 1)

(5′-AGCCGATGAAGAAGCTCTACG-3′;

(SEQ ID NO. 2)

5′ATGTCGTAGACAAGCACCTGGTA-3′);
vasa

(SEQ ID NO. 3)

(5′-ATCGAGGAGGAAATCGAGATGGA-3′;

(SEQ ID NO. 4)

5′-GGAAGCTATGCCACTGCTGAATA-3′);
gbb

(SEQ ID NO. 5)

(5′-AGATGCAGACCCTGTACATAGAC-3′;

(SEQ ID NO. 6)

5′-CTCGTCGTTCAGGTGGTACAGAA-3′);
and
rp49

(SEQ ID NO. 7)

(5′-GTATCGACAACAGAGTCGGTCGC-3′;

(SEQ ID NO. 8)

5′-TTGGTGAGCGGACCGACAGCTGC-3′).

PCR was performed as follows: at 95° C. for 4 min., 40 cycles (at 95° C. for 30 sec., at 45° C. for 30 sec., at 72° C. for 45 sec.), and at 72° C. for 7 min. RT-PCR products were electrophoresed on 2% agarose gel in the presence of ethidium bromide.

### Example 1

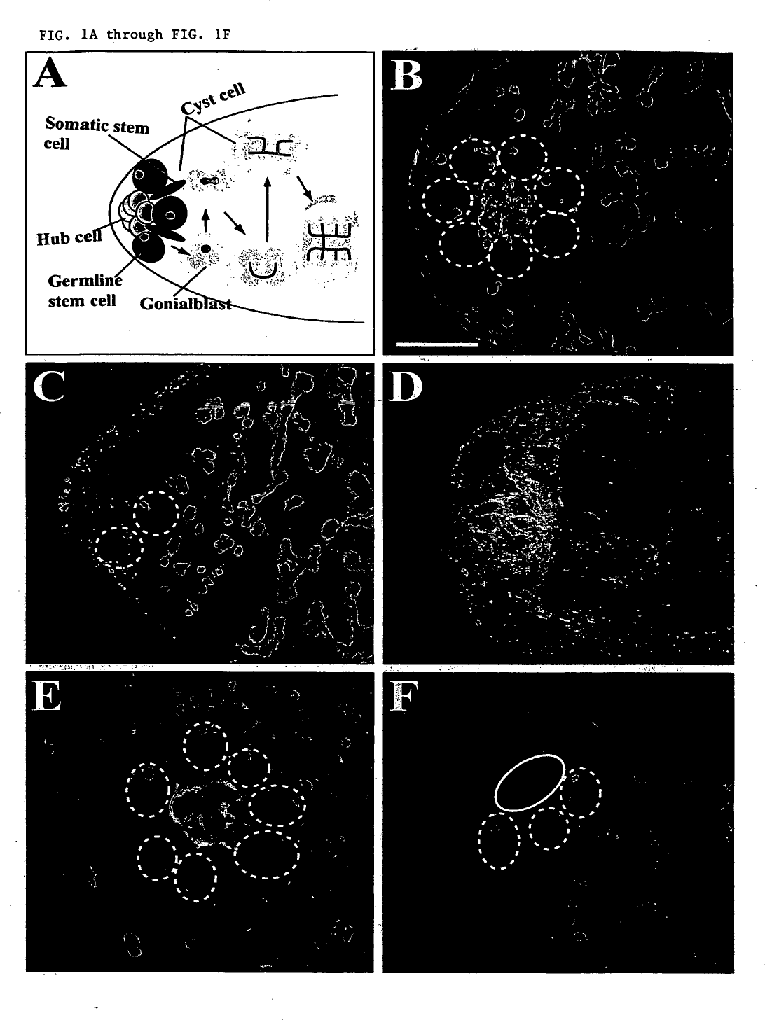

To investigate the possible role of dpp and gbb in maintaining male GSCs, GSCs were examined in the testes of temperature-sensitive dpp and gbb alleles. Two homozygous allelic combinations used in this study, gbb⁴/gbb^{D4} and gbb⁴/gbb^{D20}, were allowed to develop to adulthood at 18° C. and, subsequently, shifted to 22° C. or 25° C. for one week. An anti-Hts antibody was used to label spectrosomes and fusomes, while a DNA dye, DAPI, was used to stain nuclei. The hub was identified either by molecular markers, Fasciclin III (FasIII) or DAPI staining (small, DAPI-bright nuclei in the hub cells tightly packed together), while GSCs were identified by the presence of a spectrosome and direct contact with the hub cells. The numbers of GSCs in the testes of different mutants were quantified after the testes were immuno-stained for Hts and FasIII to visualize spectrosomes in GSCs and hub cells, respectively. A wild-type testis carried 9.1 GSCs (n=42, FIG. 1B), and these stem cells were persistent for 1 week at 25° C. or 29° C. One week after being cultured at 22° C., the testes from gbb⁴/gbb^{D4} or gbb⁴/gbb^{D20} mutants contained 1.3 (n=21) and 1.8 (n=22), respectively (FIG. 1C). One week after being cultured at 25° C., no GSCs were observed in the mutant testis (FIG. 1D). These results indicate that gbb is essential for maintaining GSCs in the testis.

Two temperature-sensitive allelic combinations, dpp^{hr4}/dpp^{hr56} and dpp⁵⁶ were used to investigate the role of dpp in maintaining GSCs in the testis. Similarly, dpp homozygous males were developed to adulthood at 18° C. and were then shifted to a restrictive temperature of 29° C. for one week. In the Drosophila ovary, both mutant combinations lost GSCs rapidly at a restrictive temperature (Xie and Spradling, 1998). Surprisingly, one week after being cultured at the restrictive temperature, the testes from Dpp mutants had no significant GSC loss. dpp^{hr4}/dpp^{hr56} and dpp^{e90}/dpp^{hr56} mutant testes had an average of 7.4 (n=44) and 8.8 (n=49) GSCs/testis, respective as shown in FIG. 1E, which is in contrast with a severe GSC loss phenotype in the dpp mutant ovary and in the Gbb mutant testis.

The two allelic dpp combinations used in this study represent weak dpp mutants. Since there is a stringent requirement for dpp during early Drosophila development, it is difficult to examine GSC loss in stronger dpp mutants since they do not survive to adulthood even at 18° C. It is still possible that the role of dpp in the maintenance of male GSCs can be revealed if gbb signaling is comprised since dpp and gbb could use the same receptors and downstream components to transduce their signals (Khalsa et al., 1998). To further study the role of dpp in the regulation of male GSCs, two mutant strains were constructed homozygous for two dpp allelic combinations that are also heterozygous for gbb: dpp^{hr4}/dpp^{hr56} gbb^{D4} and dpp^{e90}/dpp^{hr56} gbb^{D4}. The testes from the heterozygous gbb^{D4} that were cultured at 29° C. for one week had a normal GSC number of 8.6 GSCs/testis, n=38. The testes from dpp^{hr4}/dpp^{hr56} gbb^{D4} and dpp^{e90}/dpp^{hr56} gbb^{D4} had an average of 3.0 (n=13) and 5.7 (n=56 GSCs/testis, respectively (FIG. 1F) in comparison with 7.4 and 8.8 GSCs/testis for dpp mutants alone, suggesting that partial removal of gbb function can enhance the dpp mutant GSC loss phenotype in the Drosophila testis. These results indicate that dpp and gbb function cooperatively to regulate male GSCs in Drosophila.

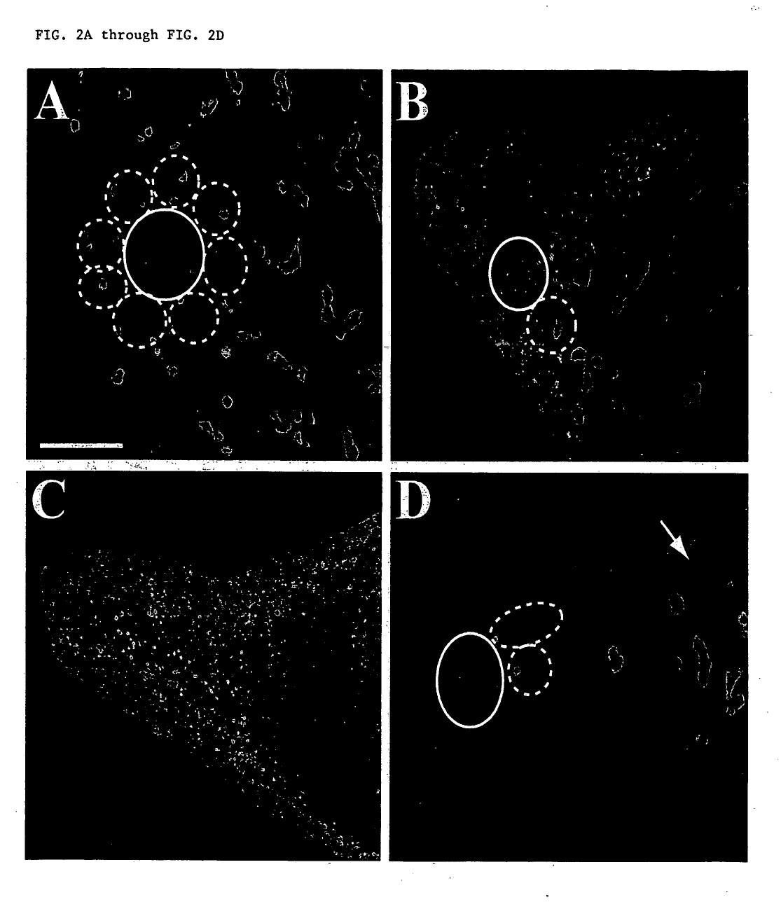

To further confirm that BMP signaling is essential for maintaining male GSCs, mutant phenotypes were studied for one of the BMP downstream components, punt, which encodes a type II serine/threonine kinase receptor for dpp and also, possibly, for gbb (Letsou et al., 1995; Ruberte et al., 1995). A punt allelic combination, punt¹⁰⁴⁶⁰/punt¹³⁵, had a temperature-sensitive feature of developing to adulthood at 18° C. and showing mutant phenotypes at 29° C. (Theisen et al., 1996). Interestingly, punt¹⁰⁴⁶⁰/punt¹³⁵ mutant males had normal GSC numbers of 8.5 GSCs/testis, n=20 after being cultured at 22° C. for a week, as shown in FIG. 2A; however, one week after shifting to 29° C., almost all the mutant testes lost their GSCs (0.1 GSCs/testis, n=58), as shown in FIGS. 2B and 2C, the wild-type testes still maintained a normal GSC number under the same conditions (data not shown). To exclude the possibility that BMP signaling was important for GSC survival, the TUNEL labeling assay was applied to look for dying GSCs in punt mutant testes. During the one-week period at 29° C., no dying GSCs were detected in the punt mutant testes but some rare dying cyst cells or differentiated germ cells were observed (n=38), as shown in FIG. 2D, suggesting that GSC loss is most likely caused by differentiation triggered by the lack of sufficient BMP signaling.

The above results further supports the idea that BMP signaling is essential for maintaining GSCs in the Drosophila testis. Two BMPs, dpp, and gbb function cooperatively to maintain GSCs in the Drosophila testis.

### Example 2

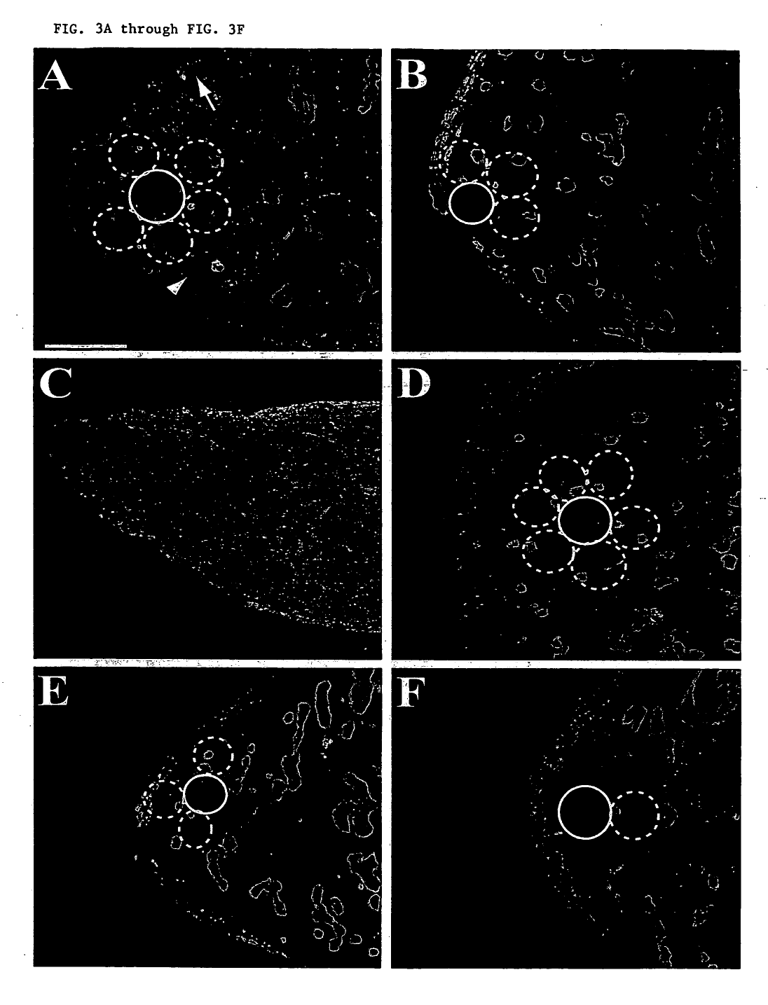

The GSC loss caused by defective BMP signaling could be due to direct and/or indirect signaling to GSCs. To investigate whether BMP signals are directly received by GSCs, the BMP signaling activities in GSCs were assessed by examining the expression of Daughters against dpp (Dad). Dad is a dpp responsive gene that negatively regulates dpp signaling (Tsuneizumi et al., 1997). Since dpp and gbb function synergistically in several developmental processes, which may result from sharing common receptors and downstream components (Haerry et al., 1998; Khalsa et al., 1998), Dad expression could potentially reflect the activation of both dpp and gbb signaling pathways. Interestingly, Dad-lacZ, which reflects Dad mRNA expression (Tsuneizumi et al., 1997), was expressed in GSCs and gonialblasts but not in more differentiated spermatogonial cells, as shown in FIG. 3A, indicating that BMP signals function as short-ranged signals, and their activities are restricted to GSCs and gonialblasts. Moreover, it was also expressed in cyst cells at higher levels but, generally, not in cyst progenitor cells, as shown in FIG. 3A.

To further determine whether mutations in gbb affect Dad expression in GSCs, the expression of Dad-lacZ in the gbb⁴/gbb^{D20} mutant background was examined. After gbb mutant males were cultured at 22° C. for four days, 97% of mutant GSCs (n=101) did not express Dad-lacZ, (as shown in FIG. 3B). Interestingly, the testes of the gbb mutant males carrying the Dad-lacZ mutation (4.8 GSCs; n=21) had more GSCs than the testes of the gbb mutant alone (2.4 GSCs; n=16). Dad-lacZ is a P element insertion into the Dad locus, and thus disrupts Dad function (Tsuneizumi et al., 1997). The removal of Dad function is known to augment dpp signaling in other developmental processes (Tsuneizumi et al., 1997). The expression of Dad-lacZ in the dpp^{hr4}/dpp^{hr56} mutant background was also examined. Even after the dpp mutant males were cultured at 29° C. for 4 days, it appeared that Dad-lacZ expression was only slightly reduced, which is consistent with no obvious GSC loss of dpp mutants in the testis. These results suggest that Dad is primarily a gbb responsive gene and also likely negatively regulates gbb signaling in the Drosophila testis.

To further test whether Dad could inhibit both gbb and dpp signaling, Dad was overexpressed in germ cells using the Gal4-UAS bipartite expression system (Brand and Perrimon, 1993). A germline-specific nanos-gal4VP16 driver can drive a target gene under the control of UAS promoter to be expressed specifically in germ cells (Van Doran et al., 1998), whereas a UAS-Dad transgene can be used to produce Dad under a gal4 driver to inhibit dpp signaling (Tsuneizumi et al., 1997). When the UAS-Dad transgene was used to overexpress Dad in germ cells by nanos-gal4VP16, all GSCs were lost in the testes before adulthood (FIG. 3C), indicating that blocking BMP signaling causes GSC loss or prevents the formation of GSCs. The GSC loss phenotype induced by Dad overexpression mimics that of gbb mutant, suggesting that Dad overexpression likely inhibits not only dpp signaling but also gbb signaling. Thus, Dad-lacZ expression in GSCs reflects the activities of both dpp and gbb signaling pathways. Together, these results suggest that BMP signals appear to function as short-range signals to control GSC maintenance through direct signaling to GSCs.

In Drosophila, dpp requires two type I receptors, thick veins (tkv) and Saxophone (sax) for signaling, which function together with the type II receptor, punt, to form receptor complexes (Brummel et al., 1994; Nellen et al., 1994; Xie et al., 1994; Letsou et al., 1995; Ruberte et al., 1995). Likely, upon dpp binding, constitutively active punt kinase activates tkv and sax kinases, which, in turn, phosphorylate Mothers against Dpp (Mad) (Newfeld et al., 1996; Newfeld et al., 1997). The phosphorylated Mad (pMad) is then associated with Medea (Med) and translocated to the nucleus to function as transcriptional activators for dpp responsive genes (Das et al., 1998; Wisotzkey et al., 1998). The expression of pMad has been directly associated with dpp signaling activity in responding cells (Tanimoto et al., 2000). To further determine whether gbb signaling is responsible for pMad expression in GSCs, the pMad expression was examined in wild-type, gbb and punt mutant GSCs in the testis. pMad preferentially accumulated in GSCs but was absent from gonialblasts and two-cell germ cell clusters, shown in FIG. 3D, which is in contrast with Dad-lacZ expression in both GSCs and gonialblasts. This difference could be due to the perdurance of lacZ mRNA and/or protein. Alternatively, levels of pMad in gonialblasts were low and could hardly be detected by the existing anti-pMad antibody. In the gbb mutant testes that still maintained some GSCs, pMad expression in the GSCs was severely reduced beyond the limits of detection, as shown in FIG. 3E. In the testes of punt¹⁰⁴⁶⁰/punt¹³⁵ mutant males cultured at the restrictive temperature, pMad levels in GSCs were severely reduced and were sometimes difficult to detect (as shown in FIG. 3F). However, pMad expression in late 16-cell germ cell clusters remained high in both the gbb and punt mutant testes. The results indicate that gbb likely signals through common BMP receptors, which leads to Mad protein phosphorylation and Dad transcription.

### Example 3

To confirm that BMP signals directly act on GSCs and control their maintenance, the FLP-mediated FRT mitotic recombination was used to generate marked GSC clones mutant for BMP downstream components (Xie and Spradling et al., 1998; Kiger et al., 2001; Tulina and Matunis, 2001). The armadillo-lacZ transgenes that are strongly expressed in all the cells in the tip of testis were used to mark mutant GSC clones. The marked GSCs were induced in adult testes by heatshock treatments and identified as lacZ-negative, spectrosome-containing germ cells that directly contact the hub cells. The percentage of testes carrying one or more marked GSCs was determined at different time points after clone induction. The rate of loss of GSCs mutant for different BMP downstream components can be used to determine how each BMP downstream component contributes to the regulation of GSCs.

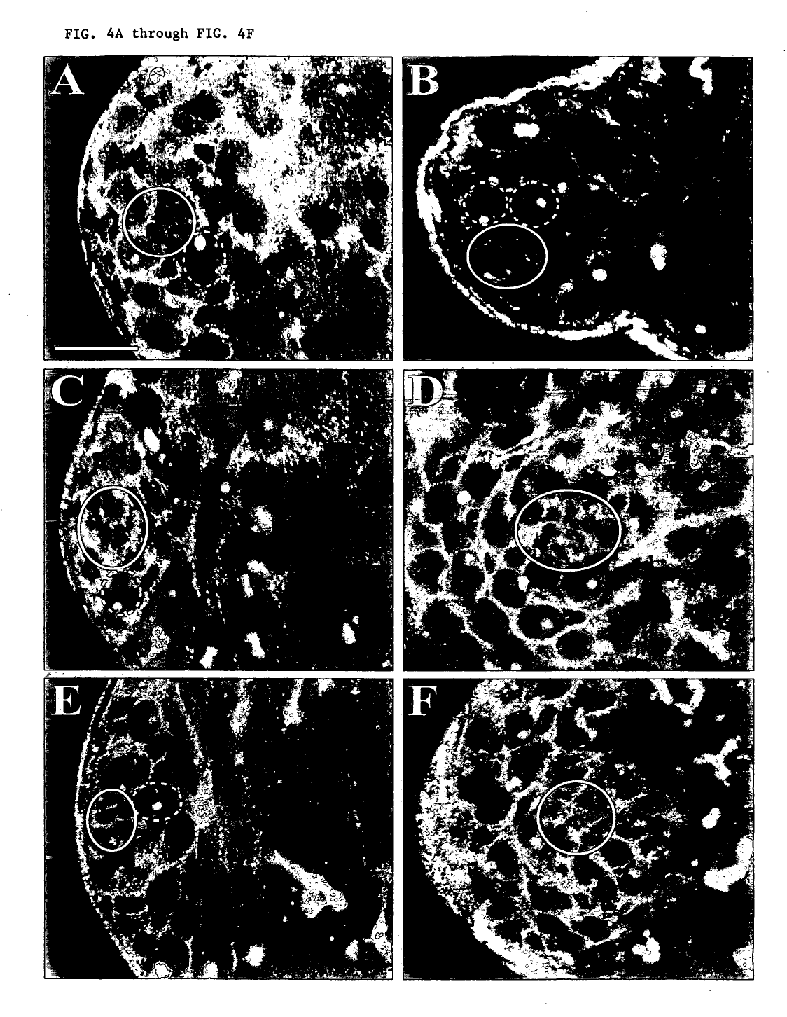

Germline stem cell clones mutant for punt tkv, sax, Mad and Med was generated according to the published procedures (Xie and Spradling, 1998) and their testes were examined beginning two days later. Two days after clone induction, 100% of the testes carried one or more marked wild-type GSCs, as shown in FIG. 4A, whereas two weeks after clone induction, 63% of the testes still carried one or more marked wild-type GSCs, shown in FIG. 4B, and Table 1 below. Two days after clone induction, over 80% of the testes still carried one or more marked GSCs mutant for tkv, sax, punt, Mad, or Med, shown in FIGS. 4C and 4E, and Table 1. In contrast to wild-type clones, marked GSC clones mutant for punt, tkv, sax, Mad, and Med were lost rapidly, shown in FIGS. 4D and 4F, and Table 1. For example, none of the testis mutant for punt¹⁰⁴⁶⁰, punt¹³⁵, tkv8, mad¹², and med²⁶ had any GSCs left two weeks after clone induction. punt¹⁰⁴⁶⁰ is a moderate allele, while the rest are strong or null alleles (Brummel et al., 1994; Nellen et al., 1994; Xie et al., 1994; Letsou et al., 1995; Ruberte et al., 1995; Das et al., 1998; and, Wisotzkey et al., 1998). Interestingly, even though sax⁴ is a strong or null sax allele (Brummel et al., 1994), two weeks after the clone induction, 6.3% of the testes carried sax mutant GSCs, indicating that sax plays a less important role in maintaining GSCs than tkv, the other type I receptor. Previous studies suggest that gbb preferentially uses sax to transduce its signal while dpp prefers tkv for its signal transduction (Haerry et al., 1998). The results show that gbb preferentially uses tkv instead of sax to transduce its signal in male GSCs. Therefore, it was concluded that BMP signals directly act on GSCs and control their maintenance in the Drosophila testis.

TABLE 1
BMP-like downstream components are required in GSCs
for their maintenance in the Drosophila testis.

| | Percentage of testes carrying one | | | | | |
|---|---|---|---|---|---|---|
| | or more marked GSC (Ages) | | | | | |
| Genotypes | Two Days | | One Week | | Two Weeks | |
| Wild Type | 100%^{a} | (73)^{b} | 81.8% | (77) | 63.6% | (55) |
| punt¹⁰⁴⁶⁰ | 92.3% | (78) | 14.6% | (89) | 0.0% | (114) |
| punt¹³⁵ | 89.1% | (92) | 21.2% | (99) | 0.0% | (149) |
| tkv⁸ | 93.3% | (90) | 1.3% | (227) | 0.0% | (149) |
| sax⁴ | 92.3% | (78) | 31.9% | (72) | 6.3% | (63) |
| Mad¹² | 82.9% | (117) | 0.7% | (140) | 0.0% | (120) |
| Med²⁶ | 92.2% | (76) | 1.9% | (107) | 0.0% | (114) |

^{a}Percentage of testes carrying marked GSCs = number of testes carrying one or more marked GSCs/total number of testes examined.
^{b}Total number of testes examined.

### Example 4

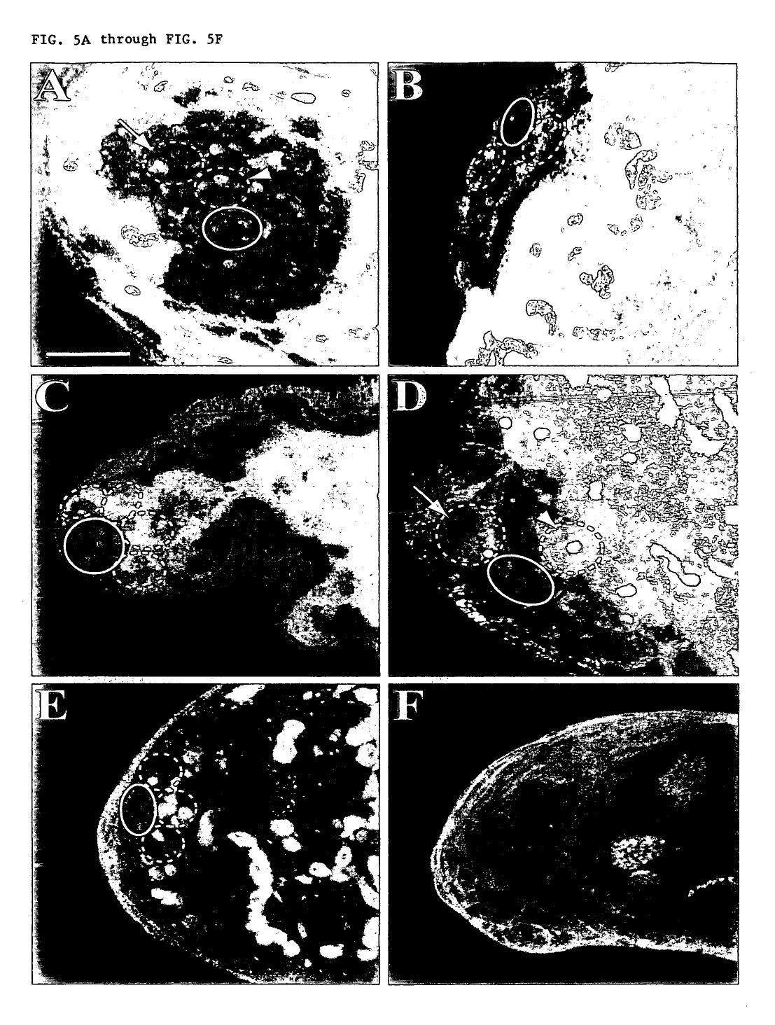

To determine whether bam transcription is repressed in male GSCs, a bam-GFP transgene was used (a bam promoter fused to the GFP gene) to examine its transcription (Chen and McKearin, 2003). Interestingly, bam was transcribed predominantly in the differentiated germ cells but not in GSCs and gonialblasts in the Drosophila testis, shown in FIG. 5A. The results that Dad is expressed only in GSCs and gonialblasts supports the idea that BMP signaling suppresses bam expression in GSCs and gonialblasts. If bam repression in GSCs is mediated by BMP signaling, it could be predicted that bam expression in GSCs defective for BMP signaling would be up-regulated. To test this prediction, dpp, gbb, or punt homozygous mutant males were generated that also carried the bam-GFP transgene for monitoring bam expression. As predicted, bam-GFP was not obviously up-regulated in dpp^{hr56}/dpp^{hr4} mutant GSCs just like in wild-type ones, consistent with the fact that the dpp mutations have little effect on the maintenance of male GSCs. Interestingly, bam-GFP expression was elevated in the gbb⁴/gbb^{D4} or gbb⁴/gbb^{D20} mutant GSCs, shown in FIG. 5B, indicating that gbb signaling is essential for repressing bam transcription in GSCs. Furthermore, at a permissive temperature of 22° C., bam expression was not detected in the punt mutant GSCs, but it was elevated inpunt mutant GSCs at a restrictive temperature of 29° C., shown in FIG. 5C. To further confirm this observation, marked mutant Med GSCs were generated that carried the bam-GFP transgene. Consistently, 66% of three-day old marked lacZ-negative mutant Med GSCs expressed bam-GFP, while neighboring unmarked lacZ-positive wild-type GSCs failed to express it, as shown in FIG. 5D. These results demonstrate that BMP signaling is required to suppress bam transcription in GSCs in the Drosophila testis.

One reason why GSCs defective in responding to BMP signals is lost are that up-regulation of bam in GSCs leads to differentiation. To further investigate whether forced bam expression in GSCs leads them to differentiate in males, two different means were used to ectopically express bam in male GSCs: hs-bam (the bam gene under the control of a hsp70 promoter) and nanos-gal4VP16 driven UAS-bam expression. Two days after heat-shock treatments (four hours per day for three days), GSCs in all the testes that did not carry hs-bam remained normal with an average of 9.3 GSCs per testis (n=26), and a week later they retained 7.6 GSCs per testis (n=30). In contrast, two days after heat-shock treatments, GSCs in most of the testes carrying hs-bam started to be reduced to an average of 3.9 GSCs per testis (n=31), shown in FIG. 5E, whereas one week later, about 70% of the testes completely lost their GSCs with an average of 2.0 GSCs (n=33), shown in FIG. 5F. Similarly, nanos-gal4-driven germ cell-specific bam expression also led all GSCs to be lost in all the testes before adulthood, whereas forced bam expression in the somatic cyst cells had no effect on GSC maintenance, supporting that bam functions in a germ cell-specific manner to trigger GSC differentiation. These results indicate that bam expression can induce male GSCs to differentiate similar to females, and further suggests that GSC loss caused by defective BMP signaling could be, at least in part, caused by elevated bam expression in GSCs.

### Example 5

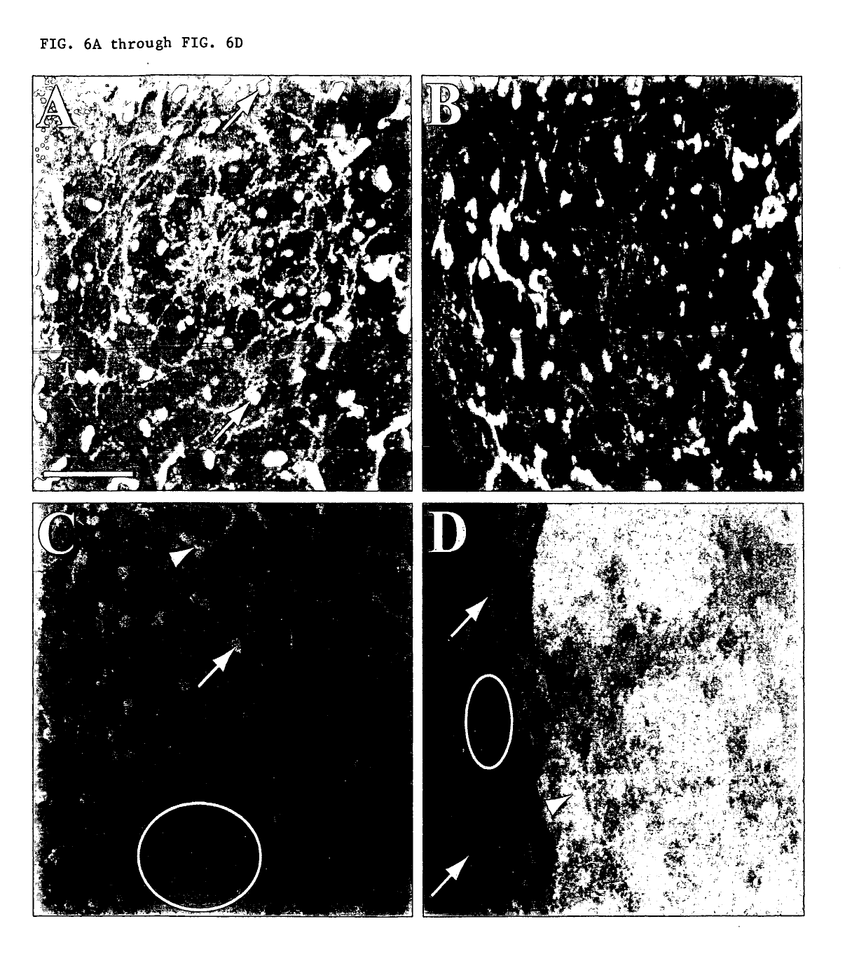

In the Drosophila ovary, dpp overexpression completely blocks germ cell differentiation, resulting in the formation of GSC-like tumors (Xie and Spradling, 1998). To determine whether dpp or gbb overexpression can also prevent germ cell differentiation in the testis, dpp or gbb was overexpressed using the nanos-gal4VP16 driver. In the testes overexpressing dpp, the hub appeared to be bigger with more cells, and there were slightly more single germ cells with a spectrosome around the hub cells, as shown in FIG. 6A, whereas gbb overexpressed testes appeared to be normal, shown in FIG. 6B. In the dpp-overexpressing testes, the gonialblasts could still differentiate and divide but failed to stop after the four rounds of cell division for normal gonialblasts, resulting in the formation of the spermatogonial clusters with more than 16 germ cells. These results suggest that overexpression of either dpp or gbb does not block gonialblast differentiation. These observations suggest that the contribution of dpp signaling to the regulation of the GSC lineage differentiation is different in males and in females. It seems that dpp or gbb overexpression directly or indirectly influences hub cell formation during early development since the nanos-gal4 driver is expressed in germ cells during early gonadal development. Likely, extra single germ cells in the dpp-overexpressing testes may be a consequence of more hub cells as the bigger hub could potentially produce more Upd molecules, which are known to influence germ cell differentiation.

In the Drosophila male, loss of bam function results in unrestricted proliferation of spermatogonial cells (Gonczy et al., 1997). In the females, dpp overexpression completely prevents bam protein accumulation in the germ cells (Xie and Spradling, 1998). Possibly, the unrestricted proliferation of spermatogonial cells in the dpp-overexpressing testis could result from the suppression of bam expression. To test whether dpp or gbb overexpression can inhibit bam expression in the testis, the nanos-gal4VP16 driver was used to overexpress dpp in the testes that also carried a bam-GFP transgene. All the germ cells, including differentiated germ cell clusters in the testes overexpressing dpp, failed to express bam-GFP, shown in FIG. 6C. However, in the testis overexpressing gbb, bam-GFP expression was repressed in some two-cell and four-cell germ cell clusters in which it is expressed in wild-type testes, but it was expressed normally in slightly older germ cells (shown in FIG. 6D). These results indicate that elevated dpp signaling, not gbb signaling, is sufficient to inhibit bam transcription in the germ cells of the testis.

### Example 6

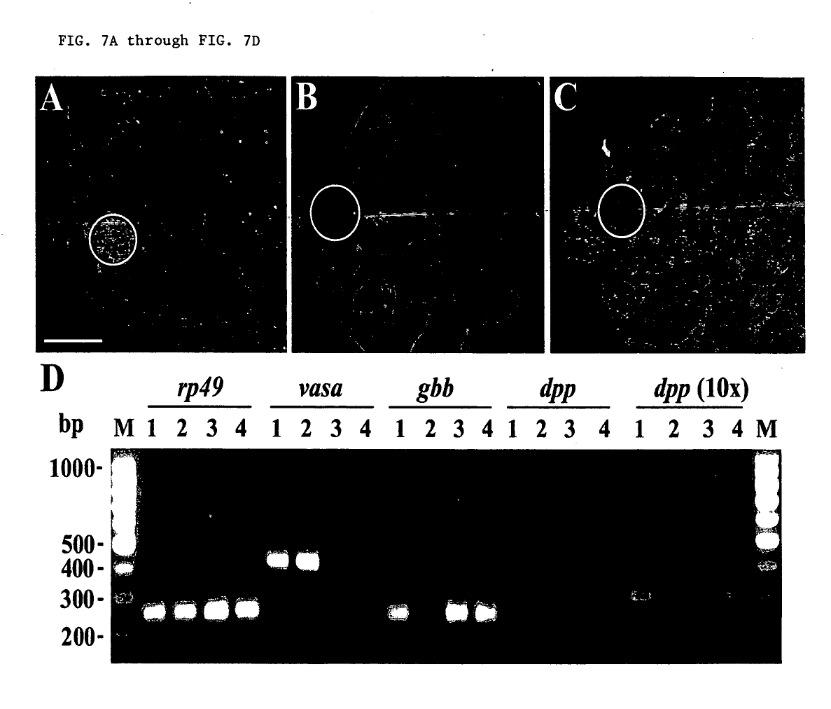

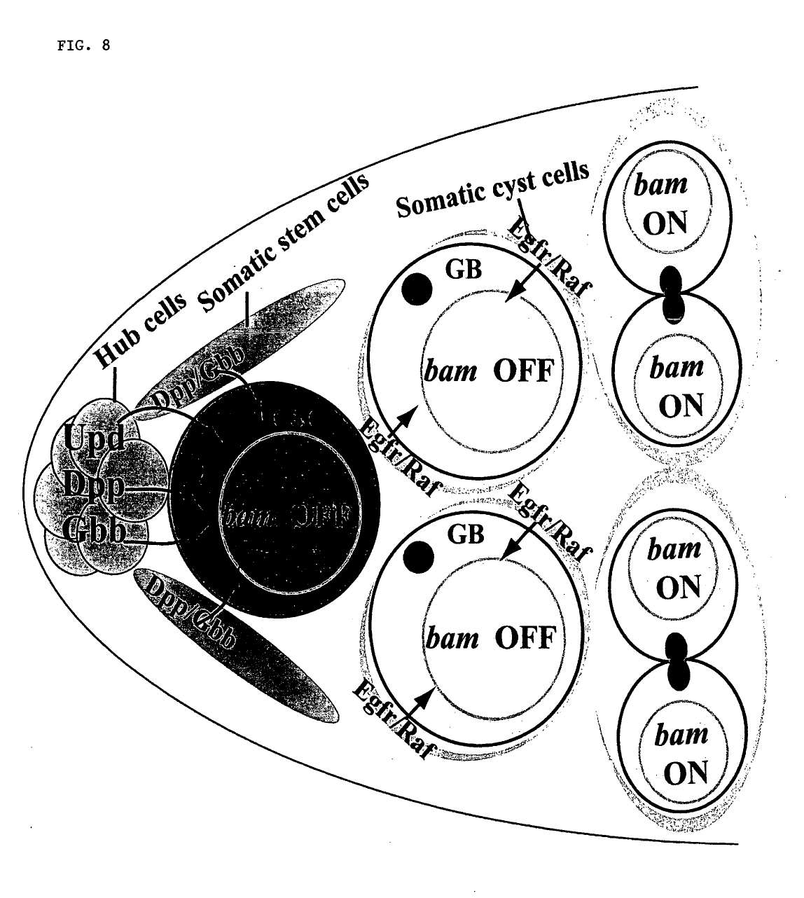

To determine the sources for gbb and dpp in the testis, RT-PCR was used to study the presence of gbb and dpp mRNAs in the purified hub cells, somatic cyst cells, and germ cells using the Fluorescence-Activated Cell Sorter (FACS). The hub cells were marked by the upd-gal4 driven UAS-GFP expression, shown in FIG. 7A. The somatic cyst cells and somatic stem cells were marked by the c587-gal4-driven UAS-GFP, shown in FIG. 7B. vasa is a germ line-specific gene (Lasko and Ashburner, 1988; Hay et al., 1988). The germ cells were marked by a vasa-GFP transgene (Nakamura et al., 2001), shown in FIG. 7C. The tips of the testes were isolated and dissociated, and the GFP-positive cells were purified from the dissociated testicular cells by FACS. As a control, vasa mRNAs were present in the whole testis and isolated germ cells, but were absent in the somatic cyst cells and hub cells, as shown in FIG. 7D. Interestingly, gbb and dpp mRNAs were present in the hub cells and the somatic cysts/somatic stem cells, but were absent in the germ cells, shown in FIG. 7D. In contrast, dpp mRNAs appeared to be less abundant, since they could only be detected when approximately 10-fold more RNA templates were used in comparison to other genes for detecting dpp, which could explain why dpp plays a less important role than gbb in the testis. These results indicate that both dpp and gbb are likely somatic cell-derived BMP signals that directly regulate GSC maintenance in the testis.

Role of BMP Niche Signaling in Drosopila Ovaries

Examples 7-13 illustrate the ability of BMP signals from niche cells to maintain germline stem cells by repressing bam transcription in Drophilia ovaries. In examples 7-13, where indicated, the following experimental procedures and reagents were employed:

Drosophila stocks and genetics—The following fly stocks used in this study were described either in FlyBase or as otherwise specified in the Results section, punt¹⁰⁴⁶⁰ and punt¹³⁵; Med²⁶; Dad-lacZ; dpp^{hr4} and dpp^{hr56}; gbb⁴, gbb^{D4}, and gbb^{D20}; bam-GFP; vasa-GFP, c587-gal4 and hs-gal4; UAS-dpp and UAS-gbb; hsFLP; FRT_{82B} armadillo-lacZ. Most stocks were cultured at room temperature. To maximize their mutant phenotypes, dpp, gbb, and punt mutant adult females were cultured at 29° C. from two days to a week. For achieving the uniform GSC-like phenotype, c587-gal4; UAS-dpp females were also cultured at 29° C. for one week.

Generating mutant GSC clones and overexpression—Clones of mutant GSCs were generated by Flp-mediated mitotic recombination, as described previously (Xu and Rubin, 1993; Xie and Spradling, 1998). To generate the stocks for making mutant GSC clones and examining bam-GFP expression, two-day old hsFLP; bam-GFP/+; FRT_{82B} Punt¹³⁵/FRT_{82B} armadillo-lacZ and hsFLP; bam-GFP/+; FRT_{82B} Med²⁶/FRT_{82B} armadillo-lacZ females were heat-shocked at 37° C. for three consecutive days with two, one-hour heat-shock treatments, separated by 8-12 hours. The ovaries were removed three days after the last heat-shock treatment and then processed for antibody staining.

To construct the stocks for overexpressing dpp or gbb, the females that carried hs-gal4 and either UAS-dpp or UAS-gbb were heat-shocked at 37° C. for different lengths of time for particular experiments as indicated in results. The females that carried c587-gal4 and UAS-dpp or UAS-gbb were cultured at room temperature or at 29° C. for one week. For examining the expression of bam-GFP in the ovary overexpressing dpp or gbb, the females that carried c587-gal4 or hs-gal4 and UAS-dpp or UAS-gbb also carried a bam-GFP transgene.

Measuring GSC loss in gbb mutants and examining bam-GFP expression in gbb, dpp or punt mutant germaria—To measure stem cell loss in gbb mutant ovaries, the germaria with different numbers of GSCs ranging from three to none were counted from the ovaries of the two-day and one-week old bam-GFP gbb⁴/gbb^{D4}, or bam-GFP gbb⁴/gbb^{D20} females. bam-GFP females carrying no gbb mutations served as a control. The 2-day old control and gbb mutant females were cultured at room temperature after they eclosed at 18° C., while the one-week old control and gbb mutant females were cultured at 29° C. Values are expressed as the average GSC number per germarium and the percentage of germaria with no GSCs.

To examine bam-GFP expression in dpp, gbb or punt mutant germaria, females were generated with the following genotypes at 18° C., bam-GFP gbb⁴/gbb^{D4}, bam-GFP gbb⁴/gbb^{D20}, bam-GFP dpp^{hr56}/dpp^{hr4} or bam-GFP; punt¹⁰⁴⁶/punt¹³⁵. bam-GFP females carrying no mutations for gbb, dpp, or punt served as a control. All the control and mutant females were cultured at 29° C. for four days before their ovaries were isolated and immunostained for comparing bam-GFP expression at identical conditions.

Immunohistochemistry—The following antisera were used, polyclonal anti-Vasa antibody (1:2000) (Liang et al., 1994); monoclonal antiHts antibody (1:3); polyclonal anti-β-galactosidase antibody (1:100) (Cappel); polyclonal anti-GFP antibody (1:200) (Molecular Probes); polyclonal anti-pMad antibody (1:200) (Tanimoto et al., 2002). The immuno-staining protocol used in this study was described previously (Song et al., 2002). All micrographs were taken using a Leica SPII confocal microscope.

Examining gene expression using the Affymetrix microarray—Total RNAs from the ovaries of different genotypes or treatments were isolated using Trizol (Invitrogen), and biotin-labeled cRNA probes were produced using an RNA transcript labeling kit (Enzo BioArray). The Drosophila GeneChips were purchased from Affymetrix Inc., and were hybridized, stained, and detected according to the handbook “GeneChip Expression Analysis” (Affymetrix Inc.).

Detecting gene expression in purified component cells using RT-PCR-After sorting GFP-positive cells with the Cytomation MoFlo, total RNAs were prepared using Trizol (Invitrogen) from the purified GFP-positive cells. The RNA samples were further amplified using the GeneChip Eukaryotic Small Sample Target Labeling Assay Version II (Affymetrix Inc.). After the RNA amplification, 100 ng of total RNAs were reverse-transcribed (RT) using SuperScriptIII First-Strand Synthesis System for RT-PCR and oligo (dT) primers according to manufacturers' protocol (Invitrogen). The following primers were used in this study:

dpp

(SEQ ID NO. 1)

(5′-AGCCGATGAAGAAGCTCTACG-3′;

(SEQ ID NO. 2)

5′-ATGTCGTAGACAAGCACCTGGTA-3′);
vasa

(SEQ ID NO. 3)

(5′ATCGAGGAGGAAATCGAGATGGA-3′;

(SEQ ID NO. 4)

5′-GGAAGCTATGCCACTGCTGAATA-3′);
gbb

(SEQ ID NO. 5)

(5′-AGATGCAGACCCTGTACATAGAC-3′;

(SEQ ID NO. 6)

5′-CTCGTCGTTCAGGTGGTACAGAA-3′;
and
rp49

(SEQ ID NO. 7)

(5′-GTATCGACAACAGAGTCGGTCGC-3′;

(SEQ ID NO. 8)

5′-TTGGTGAGCGGACCGACAGCTGC-3′.

PCR was performed as follows: 94° C. for 2 min, 35 cycles (94° C. for 30 sec, 45° C. for 30 sec, 72° C. for 45 sec), and 72° C. for 7 min RT-PCR products were electrophoresed on a 2% agarose gel in the presence of ethidium bromide.

Electrophoretic mobility shift assays for the binding of Mad and Med to the bam silencer—The GST-Mad construct was described previously (Kim et al., 1997). Med was PCR-amplified from its cDNA with the introduction of XhoI sites at both ends, then subdloned into a pGEX-4T2 vector (Amersham Pharmacia Biotech). Its sequence was confirmed by sequencing. GST-Mad, GST-Med, and GST proteins were purified by affinity chromatography using Glutathione Sepharose™ 4B according to the manufacturer's protocol (Amersham Pharmacia Biotech) and confirmed by a Western blot. Their concentrations were determined using the Bradford protein assay with BSA as a standard.

A Cy5 5′-modified oligonucleotide containing the bipartite bam silencer element (+17 to +54) was used as a probe. Binding reactions were performed according to the published protocol (Kim et al. 1997). Specificity of binding was determined by the addition of 100-fold molar excess of unlabeled competitor DNA corresponding to the bam silencer element with site A and/or B. DNA-protein complexes were resolved on a 5% (w/v) nondenaturing polyacrylamide gel using 0.5× TBE running buffer at 150 V for 3 hr at 4° C. Gels were imaged on a Typhoon 8700 (Amersham Biosciences).

### Example 7

Previous studies have shown that a dpp signal produced by somatic cells is essential for maintaining GSCs and regulating their division rate (Xie and Spradling, 1998, 2000). This signal appears to only be important for GSC maintenance but not for cystoblast development, which suggests that the function of dpp signaling is restricted to GSCs. This raises an interesting possibility that dpp only activates its signaling cascade in GSCs but not in differentiating cystoblasts. To investigate this possibility, the expression of Dad was examined in the wild-type germarium. Dad is a dpp target gene, which negatively regulates dpp signaling (Tsuneizumi et al., 1997). A Dad-lacZ enhancer trap line is often used to monitor dpp signaling in responding cells. Throughout this study, an anti-Hts antibody was used to label spectrosomes and fusomes. A DNA dye, DAPI, was also used to help identify cap cells and nuclei of other cells in the germarium. Cap cells can be reliably identified by bright DAPI staining, unique position and nuclear morphology. GSCs are identified by the presence of a spectrosome (a spherical fusome) on their anterior side and by their direct contact with cap cells. On the other hand, cystoblasts also contain a spectrosome but fail to be associated with cap cells, as shown in FIG. 9A. The germaria from the Dad-lacZ females were immuno-labeled with anti-Hts and anti-β-galactosidase antibodies to detect fusomes and the lacZ gene product, respectively. Dad-lacZ was expressed in GSCs and some cystoblasts at high levels but in the other cystoblasts and mitotic cysts at much lower levels, as shown in FIG. 9B. Interestingly, two cystoblasts in the same germarium that were at a similar distance from the cap cells had different levels of Dad-lacZ expression, suggesting that different levels of Dad expression might reflect relative ages of cystoblasts, as shown in FIG. 9B. This observation indicates that only GSCs and some cystoblasts have active dpp signaling at the highest level. Similar observations have also been made recently by Kai and Spradling (2003). These results suggest that the GSC niche is limited to the very small area adjacent to cap cells.

bam transcription is active in young, differentiating germ cells, but is repressed specifically in GSCs in the ovary (Chen and McKearin, 2003), as shown in FIG. 9C. One of the possible explanations is that dpp signaling represses bam transcription. If dpp signaling prevents bam transcription, it is predicted that the expression of bam mRNAs will be reciprocal to that of Dad. A bam-GFP transgenic line (a bam promoter fused with the GFP gene) was used to study bam transcription in the germarium, and it had normal oogenesis and a normal GSC number (Chen and McKearin, 2003), as shown in FIG. 9C. bam transcription was repressed in GSCs and some cystoblasts but was active in the other cystoblasts and dividing cystocytes, shown in FIG. 9C. To directly compare dpp signaling activity and bam transcription, the expression of bam-GFP and Dad-lacZ was examined in the same germaria. In the germ cells, including GSCs that showed strong Dad expression, there was no bam-GFP expression, shown in FIG. 9D, whereas in the germ cells, including some cystoblasts that showed weak Dad expression, bam-GFP started to be expressed, shown in FIG. 9D. The results supported the idea that dpp signaling repressed bam transcription in GSCs. The results also suggested that cystoblast differentiation was progressive and proportional to the level of bam transcription.

dpp signaling leads to the production of the phosphorylated form of Mad (pMad) through the activated Type I receptors, tkv and sax, and thus, levels of pMad accumulation also reflected levels of dpp signaling (Tanimoto et al., 2000). In the wild-type germarium, pMad was restricted to GSCs and some cystoblasts, and rapidly disappeared in other cystoblasts and two-cell cysts similar to Dad, shown in FIGS. 9E and 9F. In the same germarium, bam-GFP expression was absent in GSCs, and the cystoblasts whose pMad levels were high, while it was present in the differentiated germ cells whose pMad levels were low, FIG. 9E. The results further supported that the dpp signaling pathway is activated in GSCs at high levels, while bam transcription is actively repressed.

### Example 8

To further determine if dpp signaling is essential for repressing bam transcription in GSCs, the expression of bam-GFP in dpp mutant GSCs was investigated. The dpp^{hr56}/dpp^{hr4} temperature-sensitive mutant was chosen because it showed gradual loss of GSCs within two weeks at a restrictive temperature (29° C.) for 2 days, 4 days, or 1 week, the ovaries were immunostained with anti-GFP and anti-Hts antibodies to visualize bam-GFP and fusomes, respectively. In the germaria from the bam-GFP females, the bam-GFP expression pattern was completely normal and was absent in GSCs even one week after being cultured at 29° C., as shown in FIG. 10A. However, even two days after being cultured at 29° C., 28% of the bam-GFP dpp^{hr56}/dpp^{hr4} germaria that contained GSCs started to express bam-GFP in one or more GSCs (n=283), shown in FIG. 10B. After 4 days and 1 week, 66% (n=35) and 89% (n=19) of the mutant germaria that still had at least one GSC expressed bam-GFP in one or more GSCs, respectively, as shown in FIGS. 10C and 10D. Most of the one-week old germaria completely lost their GSCs, and the remaining germaria often had one GSC and/or started to lose the remaining GSC, shown in FIG. 10D, which is consistent with a previous study (Xie and Spradling, 1998). To further confirm the role of dpp signaling in repressing bam transcription, the levels of bam mRNAs in wild-type and dpp mutant ovaries were compared using a microarray approach. After normalization with an internal control, the actin 42A gene, bam mRNAs were dramatically up-regulated in dppₕᵣ4/dpp^{hr56} mutant ovaries in comparison with wild-type, shown in Table 2. The results demonstrated that the dpp signal is required to repress bam transcription in GSCs.

TABLE 2
dpp signaling is necessary and sufficient for repressing
Bam expression in GSCs in the Drosophila ovary

| | | hs-gal4/ | | c587-gal4/ | | dPP^{hr4}/ | |
|---|---|---|---|---|---|---|---|
| Genes | Wild Type | UAS-dpp | | UAS-dpp | | dPP^{hr56} | |
| actin 42A | 1028 | 2476^{a} | (1028)^{b} | 4686 | (1028) | 1554 | (1028) |
| dpp | 4.7 | 354.2 | (147.6) | 271.9 | (59.6) | −3.8 | (−2.5) |
| Dad | 24.0 | 103.0 | (42.9) | 296.0 | (64.9) | 4.9 | (3.2) |
| bam | 110.0 | −9.3 | (−3.8) | −3.2 | (−0.7) | 326.2 | (217.5) |

^{a}The numbers shown in this table are the arbitrary ones that are quantified by the Affymetrix scanner.
^{b}The numbers in parentheses are normalized based on the number of the housekeeping gene, actin 42A, in wild-type ovaries.

To investigate whether elevated bam transcription in dpp mutant GSCs can be correlated with reduction of pMad expression, it was determined pMad and bam expression simultaneously in the dpp mutant germaria. Both bam-GFP dpp^{hr56}/dpp^{hr4} mutant and bam-GFP females were cultured for 4 days at 29° C., which allowed dpp mutant ovaries to manifest GSC loss phenotypes. At the restrictive temperature, bam-GFP germaria maintained the normal number of GSCs and showed the normal pMAD expression pattern, shown in FIGS. 10E and 10F. In contrast, many dpp mutant germaria completely lost their GSCs, and in the remaining GSC-containing germaria in which bam-GFP was also up-regulated in GSCs, pMad was severely reduced but not completely eradicated in GSCs, shown in FIGS. 10G and 10H. In the germaria in which bam-GFP was not obviously up-regulated, pMad was relatively higher but less than normal. Those results indicated that dpp signaling contributes, at least in part, to pMad production in GSCs, and could be responsible for repressing bam transcription. There are at least two reasons that could explain why pMad is not completely eliminated in the dpp mutant GSCs. First, the dpp mutants used in this study were weak dpp mutants due to stringent dpp requirements during embryonic development. Second, another BMP-like molecule is also expressed in the germarium and is able to compensate for loss of dpp function.

### Example 9

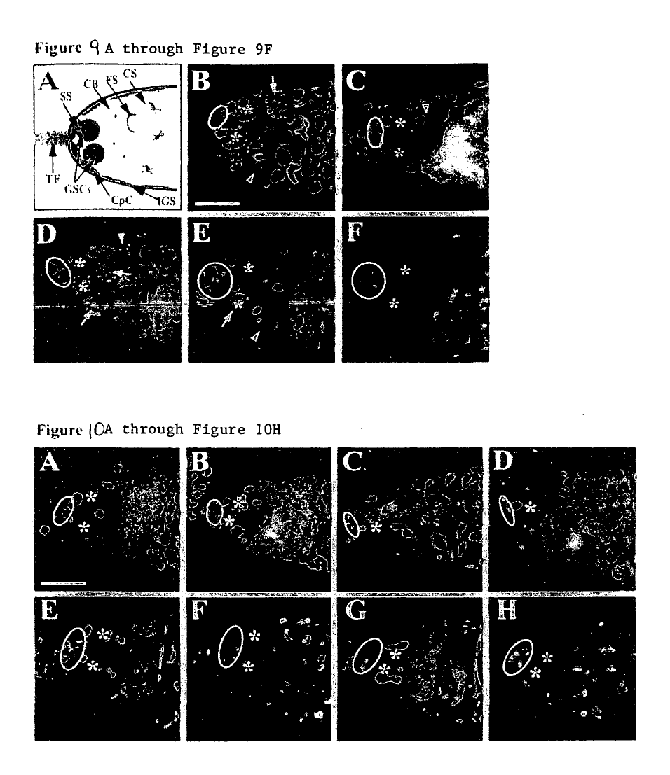

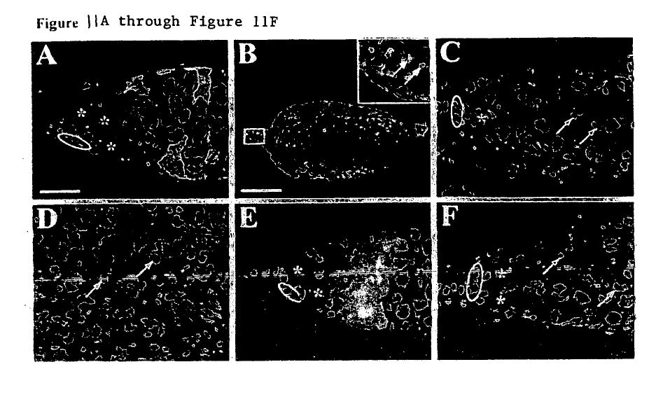

Previous studies have shown that overexpression of dpp throughout the germarium completely inhibited cystoblast differentiation and caused the accumulation of GSC-like cells that failed to express BamC (Xie and Spradling, 1998). The experiments suggested that the dpp signal was likely restricted to the tip of the germarium adjacent to cap cells. To test if GSCs-were competent to respond to dpp signaling outside their niches, dpp was specifically overexpressed in the somatic cells other than cap cells using the c587-gal4 line to drive a UAS-dpp transgene. c587-gal4 line could drive expression of a UAS-GFP transgene in inner sheath cells and follicle cells, shown in FIG. 11A. When UAS-dpp expression was driven in inner sheath cells and follicle cells, germaria were swollen and filled with single germ cells with a spectrosome, suggesting that germ cells distant from their niche are still capable of responding to dpp, shown in FIG. 11B. The reason why GSC-like cells induced by dpp overexpression fail to express BamC could be due to inhibition of bam transcription and/or translation by dpp signaling. To distinguish between these possibilities, bam-GFP expression in dpp-induced GSC-like tumors was examined. Normally, bam-GFP is expressed in most cystoblasts but not in GSCs. In the dpp-induced GSC-like tumors, bam-GFP was not expressed in the single germ cells either close to or away from, as shown in FIGS. 11C and 11D, respectively, the tip of the germarium. The results indicated that GSC-like single germ cells are competent to respond to the dpp signal outside their niche and dpp signaling is sufficient to inhibit bam transcription. The results further confirmed our previous conclusion that single germ cells with a spectrosome mostly resemble GSCs (Xie and Spradling, 1998).

The c587-gal4 driver is expressed in somatic cells during early gonadal development, and overexpression of Dpp also inhibits cystoblast differentiation at early developmental stages (Zhu and Xie, 2003). One possibility is that early dpp overexpression produces abnormal GSCs whose progeny cannot differentiate normally and fail to express bam. To exclude this possibility, bam-GFP expression was examined at adult stage when dpp was overexpressed using UAS-dpp driven by the hs-gal4 driver (the promoter of a heat-shock protein 70 gene fused with the gal4 gene). Without any heat-shock treatments, all the germaria had the normal GSC number and the normal bam-GFP expression pattern, shown in FIG. 11E. After three consecutive days of two-hour heat-shock treatments, the anterior half of the germaria were filled with single germ cells containing a spectrosome, which is consistent with our previously published results (Xie and Spradling, 1998), as shown in FIG. 11F. As expected, all the germaria showed no obvious bam-GFP expression, shown in FIG. 11F. The results further supported the idea that dpp signaling is sufficient for directly or indirectly repressing bam transcription. Due to the fact that GFP protein is stable, it was not possible to determine how fast dpp overexpression could diminish bam mRNAs using the bam-GFP transgene. To address this question, the quantity of bam mRNAs was measured two hours after a pulse of heat-shock-induced dpp overexpression using the microarray approach. Interestingly, two hours after a pulse of dpp overexpression, bam mRNA was beyond detection (Table 2), which indicated that dpp signaling rapidly repressed bam transcription and/or rendered rapid degradation of bam mRNAs. Since the response to dpp signaling was rapid, the effect on bam transcription or bam mRNA degradation was direct. This result ruled out the possibility that preventing bam transcription by dpp overexpression was an indirect consequence of ill-differentiated germ cells that failed to express bam. This result further suggested that dpp signaling might directly repress bam transcription.

### Example 10

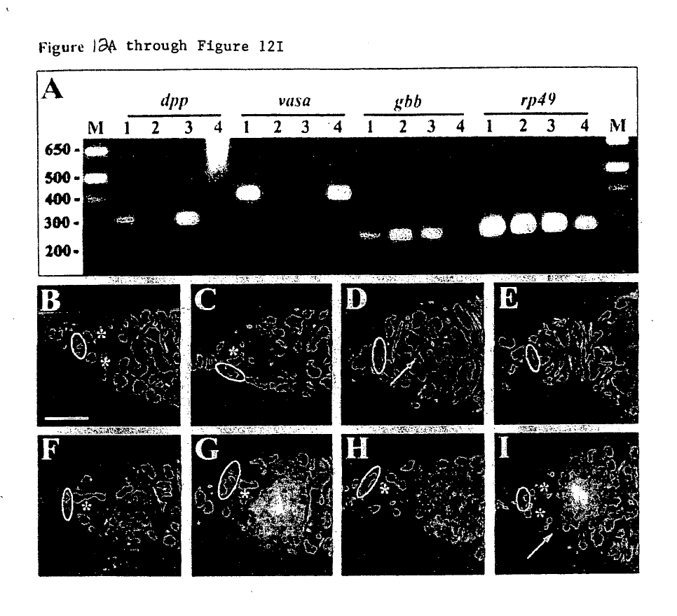

In addition to dpp, another BMP-like molecule, Glass bottom boat (gbb), exists in Drosophila and resembles human BMPs 5/6/7/8 (Wharton et al., 1991; Doctor et al., 1992). It has been shown that synergistic signaling by dpp and gbb, through the sax and tkv receptors controls wing growth and patterning in Drosophila (Khalsa et al., 1998; Haerry et al., 1998). Also, both sax and tkv are essential for maintaining GSCs in the Drosophila ovary (Xie and Spradling, 1998). To investigate the possibility that gbb could also be involved in the regulation of GSCs, it was determined whether gbb was expressed in the germarium by using RT-PCR to assay the presence of gbb mRNAs in the isolated component cells of the germarium. Inner sheath cells and early follicle cells were isolated from c587-gal4; UAS-GFP females using the fluorescence-activated cell sorter (FACS). Agametic ovaries were isolated from newly eclosed females that developed from ovo^{D1rSl} homozygous embryos lacking germ cells (Oliver et al., 1990). The agametic ovary is composed of terminal filament cells, cap cells, and early follicle cells but lacks inner sheath cells (Margolis and Spradling, 1995). Single germ cells, resembling GSCs, were isolated from c587-gal4; vasa-GFP/UAS-dpp females using the FACS. vasa-GFP is specifically expressed in the germ cells in the Drosophila ovary (Nakmura et al., 2001). vasa mRNAs were only present in germ cells but not in inner sheath cells and agametic ovaries, shown in FIG. 12A; dpp mRNAs were present in inner sheath cells and agametic ovaries but not in germ cells. FIG. 12A, indicating that different cell types in the germarium were properly isolated. vasa is a germ cell-specific gene (Lasko and Ashburner, 1988; Hay et al., 1988), and dpp is expressed in the somatic cells of the germarium but not in germ cells (Xie and Spradling, 2000). gbb mRNAs were detected in inner sheath cells and agametic ovaries but not in the GSC-like germ cells, shown in FIG. 12A, suggesting that gbb is expressed in the somatic cells. It was not possible to exclude the possibility that gbb was expressed in other differentiated germ cells in the germarium. The results indicated that gbb could be another extracellular signal for GSCs, which most likely originates from the somatic cells in the germarium. To determine whether gbb actually plays any role in the regulation of GSCs in the Drosophila ovary, the GSC number in gbb mutants was examined. Two allelic combinations of gbb, bam-GFP gbb⁴/gbb^{D4}, bam-GFP gbb⁴/gbb^{D20} and a wild-type strain carrying bam-GFP were allowed to develop to adulthood at 18° C. and were shifted to room temperature or 29° C. The germaria from the wild-type females, two days after being cultured at room temperature, or one week after being cultured at 29° C., had a normal number of GSCs, two or three GSCs, shown in FIG. 12B. On the other hand, two days after being shifted to room temperature, the germaria from the gbb⁴/gbb^{D4} and gbb⁴/gbb^{D20} females had an average of 1.0 and 1.5 GSCs, respectively (see Table 3). One week after being shifted to 29° C., 88% of the gbb⁴/gbb^{D4} mutant germaria and 60% of the gbb⁴/gbb^{D20} mutant germaria completely lost their GSCs in comparison with 36% and 7% two days after being cultured at room temperature, while the rest usually had one GSC left, as shown in FIGS. 12C-12E. The results demonstrated that gbb is essential for maintaining GSCs in the Drosophila ovary.

TABLE 3
gbb is essential for maintaining GSCs in the Drosophila ovary.

| | GSCs | |
|---|---|---|
| | 2 Days | 1 Week |
| Genotypes | (room temperature) | (29° C.) |
| bam-GFP | 2.5 ± 0.5^{a} (0.0%)^{b} (66)^{c} | 2.5 ± 0.5 (0.0%) (56) |
| bam-GFP gbb⁴/gbb^{D4} | 1.0 ± 0.8 (36.5%) (76) | 0.2 ± 0.6 (88.2%) (34) |
| bam-GFP gbb⁴/gbb^{D20} | 1.5 ± 0.7 (7.0%) (86) | 0.6 ± 0.8 (60.0%) (45) |

^{a}The numbers for averages and standard deviations are calculated using the Microsoft Excel program.
^{b}The percentage of the germaria that carry no GSCs is calculated by dividing the number of the germaria that carries no GSCs by the number of the total germaria examined.
^{c}The number of the total germaria examined for a given genotype at a particular treatment.

It has been shown that dpp was essential for repressing bam transcription. It was necessary to determine whether gbb was also essential for repressing bam transcription in GSCs. To answer this question, bam-GFP expression was examined in wild-type and gbb mutant germaria. As described earlier, bam-GFP was not expressed in GSCs in the wild-type females after being cultured at room temperature, shown in FIG. 10A. Two days after being cultured at room temperature, the GSCs rarely expressed bam-GFP in the mutant gbb⁴/gbb^{D4} germaria (one out of the total 49 germaria) and in the gbb⁴/gbb^{D20} mutant germaria (two out of the total 55 germaria), shown in FIG. 12F. By contrast, one week after being cultured at 29° C., most of the GSCs expressed bam-GFP in the gbb⁴/gbb^{D4} (5 out of the 6 germaria carrying one or more GSCs) and gbb⁴/gbb^{D20} (13 out of the 16 germaria carrying one or more GSCs) mutant germaria, as shown in FIGS. 12G and 12H. These results demonstrated that gbb was also essential for repressing bam transcription in GSCs.

To determine if gbb overexpression was sufficient for repressing bam transcription in germ cells like dpp, bam-GFP expression was examined in the strain carrying c587-gal4, UAS-gbb, and bam-GFP transgenes, in which gbb was overexpressed in the germarium. The UAS-gbb transgene had been used to effectively overexpress gbb in the wing disc (Khalsa et al., 1998). The germaria overexpressing gbb had the normal number of GSCs and cysts, which is shown in FIG. 12I, indicating that GSC maintenance, division, and germ cell differentiation appear to be normal. Similarly, the bam-GFP expression pattern was also normal in the gbb-overexpressing germarium as it had in the wild-type germarium, shown in FIG. 12I. The results indicated that gbb overexpression, unlike that of dpp, was not sufficient to inhibit bam transcription.

### Example 11

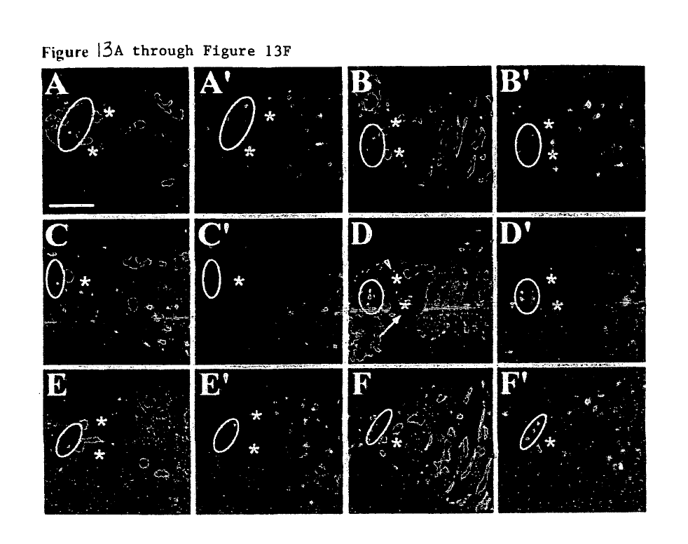

It is apparent that gbb uses the same downstream components as dpp does in regulating wing development (Khalsa et al., 1998; Haerry et al., 1998). Thus, gbb could also potentially use the same downstream components to regulate GSC maintenance as dpp does. dpp signaling results in the activation of type I receptors, tkv and sax, that in turn phosphorylate Mad (Newfeld et al., 1996; Tanimoto et al., 2000). To investigate whether gbb was also involved in the production of pMad in GSCs, the pMad accumulation in gbb mutant GSCs was examined, and the relationship between pMad accumulation and bam transcription was examined. As expected, pMad and bam-GFP expression patterns in GSCs and cystoblasts remained normal four days after the females were cultured at 29° C. Levels of pMad were high and bam-GFP was not expressed in GSCs, which is shown in FIGS. 13A and 13A′. Four days after being cultured at 29° C., the expression of pMad in the GSCs in both gbb⁴/gbb^{D4} and gbb⁴/gbb^{D20} females was generally reduced, shown in FIGS. 13B to 13F′. Although some of the mutant gbb germaria had reduced pMad expression in GSCs, in these germaria, bam-GFP remained unexpressed in GSCs, shown in FIGS. 13B to 13D′. The other germaria had severely reduced levels of pMad in GSCs, and in those germaria, bam-GFP was obviously up-regulated in GSCs, shown in FIGS. 13E to 13F′. There appeared to be a good correlation between levels of pMad and bam-GFP expression in gbb mutant GSCs. The results indicated that gbb signaling also resulted in the phosphorylation of Mad, likely through activating tkv and sax receptors, and that levels of pMad in GSCs seem to correlate with levels of bam repression.

The study showed that both dpp and gbb are required for the production of pMad and for repressing bam transcription in GSCs. However, some of the GSCs mutant for either dpp or gbb still retained detectable levels of pMad. To further investigate whether dpp and gbb function synergistically to regulate GSC maintenance by repressing bam transcription, dpp and gbb double mutants were generated, homozygous dpp mutants that were heterozygous for gbb, homozygous gbb mutants that were heterozygous for dpp, and transheterozygotes for dpp and gbb. Only dpp and gbb transheterozygous females were recovered, while the females with the other genotypes had never been recovered, even at 18° C. the germaria from the dpp and gbb transheterozygous females had similar numbers of GSCs to that of dpp or gbb heterozygotes at room temperature or at 29° C. Therefore, it could not be directly determined whether dpp and gbb functioned synergistically. Since mutations in either dpp or gbb affect pMad accumulation in GSCs, it is very likely that dpp and gbb function cooperatively.

### Example 12

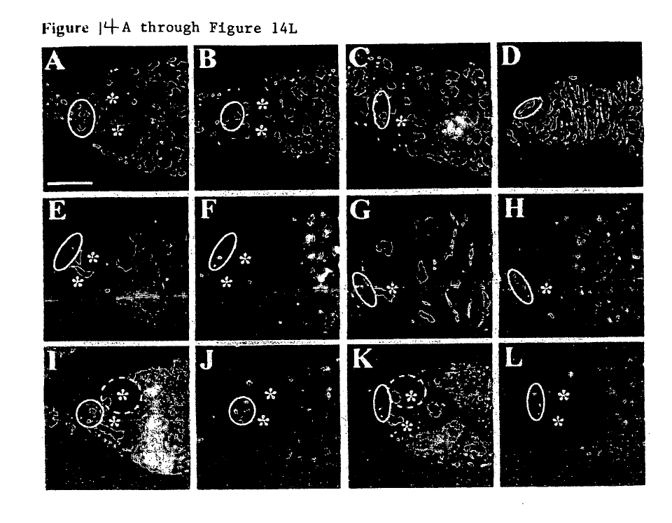

To further determine whether BMP downstream components were required for repressing bam transcription, bam-GFP expression was examined in punt mutant ovaries. punt encodes a Type II serine/threonine kinase receptor for dpp and also, possibly, for gbb (Letsou et al., 1995; Ruberte et al., 1995). A temperature-sensitive punt allelic combination, punt¹⁰⁴⁶⁰/punt¹³⁵, can develop to adulthood at 18° C. and exhibit mutant phenotypes at 29° C. (Thiesen et al., 1996). Newly eclosed punt¹⁰⁴⁶⁰/punt¹³⁵ females at 18° C. had a normal number of GSCs and a normal bam-GFP expression pattern in the germarium, shown in FIG. 14A. Consistent with the fact that marked GSC clones mutant for punt¹⁰⁴⁶⁰ or punt¹³⁵ are lost rapidly (Xie and Spradling, 1998), some punt¹⁰⁴⁶⁰/punt¹³⁵ mutant GSCs started to express bam-GFP two days after being shifted to 29° C., shown in FIG. 14B. One week after being cultured at 29° C., the GSCs in 75% of the mutant germaria (a total of 97 germaria examined) that still carried one or more GSCs had already expressed bam-GFP, shown in FIG. 14C, and 53% of the mutant germaria (a total of 123 germaria examined) only had one or no GSC, as shown in FIGS. 14C and 14D. To determine the relationship between bam-GFP expression and pMad accumulation, bam-GFP and pMad expression were examined in punt mutant germaria four days after being cultured at 29° C. After four days at the restrictive temperature, pMad in most punt mutant GSCs was severely reduced and bam-GFP was also up-regulated, shown in FIGS. 14E-14H. The results further demonstrated that defective BMP signaling resulted in the de-repression of bam transcription in GSCs and that levels of pMad are correlated with the repression status of bam transcription in GSCs.

To determine whether direct BMP signaling is necessary for repressing bam transcription in GSCs, marked GSCs were generated which were mutant for punt¹³⁵ and Med²⁶, and then bam-GFP expression was examined in the marked mutant GSCs. Med encodes a common Smad 4 for TGF-β-like signaling pathways, and Mea²⁶ is a strong Med mutant (Das et al., 1998; Wisotzkey et al., 1998). punt¹³⁵ is a strong punt mutant (Letsou et al., 1995; Ruberte et al., 1995). The previous study indicated that most of the marked mutant GSCs for punt¹³⁵ and Med²⁶ were lost within one week (Xie and Spradling, 1998). bam-GFP expression was examined in three-day old marked mutant GSCs, with 54% of the marked punt¹³⁵ GSCs expressing bam-GFP (a total of 37 marked GSC clones examined) and 65% of the marked Med²⁶ mutant GSCs showing obvious bam-GFP up-regulation (a total of 48 marked GSC clones examined, shown in FIGS. 14I-14L. The results demonstrated that direct BMP signaling is necessary for repressing bam transcription.

### Example 13

It has been shown that BMP signaling mediated by dpp and gbb is essential for repressing bam transcription in GSCs and, thus, maintaining GSC identity. This bam transcriptional repression could be directly or indirectly controlled by BMP signaling. As shown recently by Chen and McKearin (2003), the repression of bam transcription in GSCs is mediated through a silencer located at the 5′ UTR of the bam gene, which is composed of two sites, A and B. In several developmental processes, the brinker (brk) gene is actively repressed by dpp signaling (Campbell and Tomlinson, 1999; Jazwinska et al., 1999; Minami et al., 1999; Marty et al., 2000), and the repression is also controlled by a silencer (Muller et al., 2003). Interestingly, bam and brk silencers show remarkably similar sequences, with 13 out of 19 base pairs identical in A and B sites, as shown in FIG. 15A. The brk silencer has been shown to be directly occupied by a complex containing Mad, Med, and Schnurri (shn), and its repression requires shn and functional dpp signaling (Muller et al., 2003). shn is known to be required in GSCs for their maintenance, and loss of shn function results in GSC loss (Xie and Spradling, 2000). All the evidence suggests that the bam silencer could be directly occupied by a complex containing Mad, Med and, possibly, shn.

The A and B sites of the bam silencer were together sufficient for repressing a constitutive germ cell-specific promoter in GSCs (Chen and McKearin, 2003), shown in FIGS. 15A and 15B. To determine whether Med and Mad can bind directly to the defined bam silencer in vitro, a Cy5-labeled DNA fragment was used containing the bam silencer and purified bacterially expressed GST-Mad and GST-Med to perform electrophoretic mobility shift assays. GST-N-Mad and GST-Med were purified to homogeneity, shown in FIG. 15C. GST-N-Mad (a fusion between GST and the N-terminal DNA binding domain and linker region of Mad) was shown to bind to the dpp responsive elements in vitro (Kim et al., 1997), whereas GST-med is a fusion of GST with the full-length Med. Interestingly, both Mad and Med could bind to the silencer but with different affinities. It appeared that Med bound to the silencer with a higher affinity than Mad, shown in FIG. 15D. The binding specificity of Mad and Med to the silencer was demonstrated by a competition experiment with an unlabeled DNA fragment containing A and B sites, shown in FIG. 15D. The unlabeled DNA fragment containing either A or B site could almost completely compete for binding of Mad to the labeled silencer. However, the unlabeled DNA fragment with the A site, but to much less extent with the B site, could compete for binding of Med to the labeled silencer. The data suggested that Mad occupied both A and B sites, while Med preferentially binds to the A site. pMad accumulates in the GSC nucleus at high levels (Kai and Spradling, 2003). As described, Med is also required in GSCs for repressing bam transcription. The in vitro binding results suggested that a protein complex containing Mad and Med stimulated by BMP signaling directly binds to the bam silencer to repress its transcription in GSCs.

### CONCLUSION

It has been shown that BMP signals, dpp and gbb, from somatic cellsare essential for maintaining GSCs in the Drosophila testis. It was also shown that the gbb signal was essential for keeping bam repressed in GSCs and that forced bam expression in GSCs leads to differentiation in the testis. Moreover, similarities between Drosophila males and females with regards to GSC regulation were elucidated. In particular, both male and female GSCs require BMP signaling for their maintenance and for repressing bam transcription in GSCs. In summary, therefore, novel insights into understanding how a niche signal controls stem cell self-renewal and into understanding how the same signals in different niches function similarly with regard to the regulation of GSCs were revealed.

Accordingly, methods and compositions have been described that may be employed to promote stem cell differentiation. Moreover, methods and compositions have been described that may be utilized to maintain stem cells in an undifferentiated state. It is apparent to those skilled in the art, however, that many changes, variation, modifications, and other uses and applications for the invention described herein are possible. Moreover, these changes, variations, modifications, and other uses do not depart from the spirit and scope of the invention.

All references cited in the preceding text of the patent application or in the following reference list, to the extent that they provide exemplary, procedural, or other details supplementary to those set forth herein, are specifically incorporated by reference to the same extent as if each individual publication or patent application was specifically and individually indicated to be incorporated by reference.

### REFERENCE LIST

Arora, K., M. S. Levine, and M. B. O'Connor. 1994. The screw gene encodes a ubiquitously expressed member of the TGF-beta family required for specification of dorsal cell fates in the Drosophila embryo. Genes & Dev. 8: 2588-2601.
Biehs, B., V. Francois and E. Bier. 1996. The Drosophila short gastrulation gene prevents Dpp from autoactivating and suppressing neurogenesis in the neuroectoderm. Genes & Dev. 10: 2922-2934.
Brand, A. H. and N. Perrimon. 1993. Targeted gene expression as a means of altering cell fates and generating dominant phenotypes. Development 118: 401-415.
Brummel, T. J., V. Twombly, G. Marques, J. L. Wrana, S. J. Newfeld, L. Attisano, J. Massague, M. B. O'Connor, and W. M. Gelbart. 1994. Characterization and relationship of Dpp receptors encoded by the saxophone and thick veins genes in Drosophila. Cell 78: 251-261.
Campbell, G. and A. Tomlinson. 1999. Transducing the Dpp morphogen gradient in the wing of Drosophila: regulation of Dpp targets by Brinker. Cell 96: 553-562.
Chen, D. and D. M. McKearin. 2003. A discrete transcriptional silencer in the bam gene determines asymmetric division of the Drosophila germline stem cell. Development 130: 1159-1170.
Cox, D. N., A. Chao, and H. Lin. 2000. Piwi encodes a nucleoplasmic factor whose activity modulates the number and division rate of germline stem cells. Development 127: 503-514.
Cox, D. N., A. Chao, J. Baker, L. Chang, D. Qiao and H. Lin. 1998. A novel class of evolutionarily conserved genes defined by piwi are essential for stem cell self-renewal. Genes Dev. 12: 3715-3727.
Das, P., L. L. Maduzia, H. Wang, A. L. Finelli, S. H. Cho, M. M. Smith, and R. W. Padgett. 1998. The Drosophila gene Medea demonstrates the requirement for different classes of Smads in dpp signaling. Development 125: 1519-1528.
de Cuevas, M., M. A. Lilly, and A. C. Spradling. 1997. Germline cyst formation in Drosophila. Annu. Rev. Genet. 31: 405-428.
Doctor, J. S., P. D. Jackson, K. E. Rashka, M. Visalli, and F. M. Hofmann. 1992. Sequence, biochemical characterization, and developmental expression of a new member of the TGF-beta superfamily in Drosophila melanogaster. Dev. Biol. 151: 491-505.
Forbes, A. and R. Lehmann. 1998. Nanos and Pumilio have critical roles in the development and function of Drosophila germline stem cells. Development 125: 679-690.
Gonczy, P., E. Matunis and S. DiNardo. 1997. bag-of-marbles and benign gonial cell neoplasm act in the germline to restrict proliferation during Drosophila spermatogenesis. Development 124: 4361-4371.
Haerry, T. E., O. Khalsa, M. B. O'Connor, and K. A. Wharton. 1998. Synergistic signaling by two BMP ligands through the SAX and TKV receptors controls wing growth and pattering in Drosophila. Development 125: 3977-3987.
Hay, B., L. Y. Jan, and Y. N. Jan. 1988. A protein component of Drosophila polar granules is encoded by vasa and has extensive sequence similarity to ATP-dependent helicases. Cell 55: 577-587.
Jazwinska, A., N. Kirov, E. Wieschaus, S. Roth and C. Rushlow. 1999. The Drosophila gene brinker reveals a novel mechanism of Dpp target gene regulation. Cell 96: 563-573.
Kai, T. and A. Spradling. 2003. An empty Drosophila stem niche cell reactivates the proliferation of ectopic cells. Proc. Natl. Acad. Sci. USA 100: 4633-4638.
Khalsa, O., J. W. Yoon, S. Torres-Schumann, and K. A. Wharton. 1988. TGF-β/BMP superfamily members, Gbb-60A and Dpp, cooperate to provide pattern information and establish cell identity in the Drosophila wing. Development 125: 2723-2734.
Kiger, A. A., D. L. Jones, C. Schulz, M. B. Rogers, and M. T. Fuller. 2001. Stem cell self-renewal specified by JAK-STAT activation in response to a support cell cue. Science 294: 2542-2545.
Kiger, A. A., H. White-Cooper, and M. T. Fuller. 2000. Somatic support cells restrict germline stem cell self-renewal and promote differentiation. Nature 407: 750-754.
Kim, J., K. Johnson, H. J. Chen, S. Carroll and A. Laughon. 1997. Drosophila Mad binds to DNA and directly mediates activation of vestigial by Decapentaplegic. Nature 388: 304-308.
King, F. J. and H. Lin. 1999. Somatic signaling mediated by fs(1)Yb is essential for germline stem cell maintenance during Drosophila oogenesis. Development 126: 1833-1844.
King, F. J., A. Szakmary, D. N. Cox, and H. Lin. 2001. Yb modulates the divisions of both germline and somatic stem cells through piwi- and hh-mediated mechanisms in the Drosophila ovary. Mol. Cell 7: 497-508.
Lasko, P. F. and M. Ashburner. 1988. The product of the Drosophila gene vasa is very similar to eukaryotic initiation factor-4A. Nature 335: 611-617.
Lavoie, C. A., B. Ohlstein, and D. M. McKearin. 1999. Localization and function of Bam protein require the benign gonial cell neoplasm gene product. Dev. Biol. 212: 405-413.
Letsou, A., K. Arora, J. L. Wrana, K. Simin, V. Twombly, J. Jamal, K. Staehling-Hampton, F. M. Hoffmann, W. M. Gelbart, J. Massague, and et al. 1995. Drosophila DPP signaling is mediated by the punt gene product: a dual ligand-binding type II receptor of the TGF beta receptor family. Cell 80: 899-908.
Lin, H. and A. C. Spradling. 1997. A novel group of pumilio mutations affects the asymmetric division of germline stem cells in the Drosophila ovary. Development 124: 2463-2476.
Lin, H., L. Yue, and A. C. Spradling. 1994. The Drosophila fusome, a germline-specific organelle, contains membrane skeletal proteins and functions in cyst formation. Development 120: 947-956.
Lin, H. 2002. The stem-cell niche theory: lessons from flies. Nat. Rev. Genet. 3: 931-940.
Margolis, J. and A. Spradling. 1995. Identification and behavior of epithelial stem cells in the Drosophila ovary. Development 121: 3797-3807.
Marques, G., M. Musacchio, M. J. Shimmel, K. Wunnenberg-Stapleton, K. W. Cho and M. B. O'Connor. 1997. Production of a DPP activity gradient in the early Drosophila embryo through the opposing actions of the SOG and TLD proteins. Cell 91: 417-426.
Marty T., B. Muller, K. Basler and M. Affolter. 2000. Schnurri mediates Dpp-dependent repression of brinker transcription. Nature Cell Biol. 2: 745-749.
Massague, J. and D. Wotton. 2000. Transcriptional control by the TGF-beta/Smad signaling system. EMBO J. 19:1745-1754.
Matunis, F. J. Tran, P. Gonczy, K. Caldwell and S. DiNardo. 1997. punt and schnurri regulate a somatically derived signal that restricts proliferation of committed progenitors in the germline. Development 124: 4383-4391.
McKearin, D. and B. Ohlstein. 1995. A role for the Drosophila bag-of-marbles protein in the differentiation of cystoblasts from germline stem cells. Development 121: 2937-2947.
McKearin D. M. and A. C. Spradling. 1990. Bag-of-marbles: a Drosophila gene required to initiate both male and female gametogenesis. Genes & Dev. 4: 2242-2251.
Minami, M., N. Kinoshita, Y. Kamoshida, H. Tanimoto and T. Tabata. 1999. Brinker is a target of Dpp in Drosophila that negatively regulates Dpp-dependent genes. Nature 398: 242-246.
Muller, B., B. Hartmann, G. Pyrowolakis, M., Affolter and K. Basler. 2003. Conversion of an extracellular DPP/BMP morphogen gradient into an inverse transcriptional gradient. Cell 113: 221-233.
Nakamura, A., R. Amikura, K. Hanyu, and S. Kobayashi. 2001. Me31B silences translation of oocyte-localizing RNAs through the formation of cytoplasmic RNP complex during Drosophila oogenesis. Development 128: 3233-3242.
Nellen, D., M. Affolter, and K. Basler. 1994. Receptor serine/threonine kinases implicated in the control of Drosophila body pattern by decapentaplegic. Cell 78: 225-237.
Newfeld, S. J., E. H. Chartoff, J. M. Graff, D. A. Melton, and W. M. Gelbart. 1996. Mothers against dpp encodes a conserved cytoplasmic protein required in DPP/TGF-β responsive cells. Development 122: 2099-2108.
Newfeld, S. J., A. Mehra, M. A. Singer, J. L. Wrana, L. Attisano, and W. M. Gelbart. 1997. Mothers against dpp participates in a DDP/TGF-β responsive serine-threonine kinase signal transduction cascade. Development 124: 3167-3176.
Ohlstein, B. and D. McKearin. 1997. Ectopic expression of the Drosophila Bam protein eliminates oogenic germline stem cells. Development 124: 3651-3662.
Oliver, B., D. Pauli and A. P. Mahowald. 1990. Genetic evidence that the ovo locus is involved in Drosophila germ line sex determination. Genetics 125: 535-550.
Padgett, R. W., R. D. St Johnston, and W. M. Gelbart. 1987. A transcript from a Drosophila pattern gene predicts a protein homologous to the transforming growth factor-beta family. Nature 325: 81-84.
Podos, S. D. and E. L. Ferguson. 1999. Morphogen gradients: new insights from DPP. Trends Genet. 15: 396-402.
Rajan P, D. M. Panchision, L. F. Newell and R. D. McKay. 2003. BMPs signal alternately through a SMAD or FRAP-STAT pathway to regulate fate choice in CNS stem cells. J Cell Biol. 161: 911-921.
Ruberte, E., T. Marty, D. Nellen, M. Affolter, and K. Basler. 1995. An absolute requirement for both the type II and type I receptors, punt and thick veins, for dpp signaling in vivo. Cell 80: 889-897.
Song, X., Ch. H. Zhu, C. Doan, and T. Xie. 2002. Germline stem cells anchored by adherens junctions in the Drosophila ovary niches. Science 296: 1855-1857.
Spradling, A., D. Drummond-Barbosa, and T. Kai. 2001. Stem cells find their niche. Nature 414: 98-104.
Tanimoto, H., S. Itoh, P. ten Dijke, and T. Tabata. 2000. Hedgehog creates a gradient of DPP activity in Drosophila wing imaginal discs. Mol. Cell5: 59-71.
Theisen, H., T. E. Haerry, M. B. O'Connor, and J. L. Marsh. 1996. Developmental territories created by mutual antagonism between Wingless and Decapentaplegic. Development 122: 3939-3948.
Torres-Vazquez, J., S. Park, R. Warrior and K. Arora. 2001. The transcription factor Schnurri plays a dual role in mediating Dpp signaling during embryogenesis. Development 128: 1657-1670.
Tran, J., T. J. Brenner, and S. DiNardo. 2000. Somatic control over the germline stem cell lineage during Drosophila spermatogenesis. Nature 407: 754-757.
Tsuneizumi, K., T. Nakayama, Y. Kamoshida, T. B. Kornberg, J. L. Christian, and T. Tabata. 1997. Daughters against dpp modulates dpp organizing activity in Drosophila wing development. Nature 389: 627-631.
Tulina, N. and E. Matunis. 2001. Control of stem cell self-renewal in Drosophila spermatogenesis by JAK-STAT signaling. Science 294: 2546-2549.
Van Doren, M., A. L. Williamson, and R. Lehmann. 1998. Regulation of zygotic gene expression in Drosophila primordial germ cells. Curr. Biol. 8: 243-246.
Watt, F. M. and B. L. Hogan. 2000. Out of Eden: stem cells and their niches. Science 287: 1427-1430.
Wharton, K. A., G. H. Thomsen, and W. M. Gelbart. 1991. Drosophila 60A gene, another transforming growth factor β family member, is closely related to human bone morphogenetic proteins. Proc. Natl. Acad. Sci. USA 88: 9214-9218.
Wisotzkey, R. G., A. Mehra, D. J. Sutherland, L. L. Dobens, X. Liu, C. Dohrrmann, L. Attisano, and L. A. Raftery. 1998. Medea is a Drosophila Smad4 homolog that is differently required to potentiate DPP responses. Development 125: 1433-1445.
Xie, T., A. L. Finelli, and R. W. Padgett. 1994. The Drosophila saxophone gene: a serine-threonine kinase receptor of the TGF-beta superfamily. Science 263: 1756-1759.
Xie, T. and A. C. Spradling. 1998. Decapentaplegic is essential for the maintenance and division of germline stem cells in the Drosophila ovary. Cell 94: 251-260.
Xie, T. and A. C. Spradling. 2000. A niche maintaining germ line stem cells in the Drosophila ovary. Science 290: 328-330.
Xie, T. and A. C. Spradling. 2001. The Drosophila ovary: an in vitro stem cell system. In Stem Cell Biology (ed. D. R. Marshak, R. L. Gardner, and D. Gottlieb), pp. 129-148. Cold Spring Harbor Laboratory Press, Cold Spring Harbor, N.Y.
Xu, T. and G. M. Rubin. 1993. Analysis of genetic mosaics in developing and adult Drosophila tissues. Development 117: 1223-1237.
Zhu, C. H., and T. Xie, 2003. Clonal expansion of ovarian germline stem cells during niche formation in Drosophila. Development 130: 2579-2588.

1. A method for maintaining the undifferentiated state of an isolated stem cell, the method comprising:
(a) providing an isolated stem cell;
(b) contacting in vitro, the stem cell with an isolated stem niche cell such that a molecule expressed from the stem niche cell activates a signal transduction cascade in the stem cell causing repression of the expression of a gene in the stem cell necessary for differentiation of a stem daughter cell.

2. The method of claim 1, wherein the stem cell is a cultured cell.

3. The method of claim 1, wherein the stem cell and stem niche cell are isolated from an organism that is either a vertebrate or an invertebrate.

4. The method of claim 3, wherein the vertebrate is a mammal.

5. The method of claim 4, wherein the mammal is a human.

6. The method of claim 3, wherein the invertebrate is an insect.

7. The method of claim 6, wherein the insect is a Drosophila.

8. The method of claim 1, wherein the stem cell is either a somatic stem cell or a germline stem cell.

9. The method of claim 1, further comprising, maintaining the stem cell in either a pluripotent state or a totipotent state.

10. The method of claim 1, wherein the stem niche cell comprises at least one somatic cell selected from the group consisting of a terminal filament cell, a cap cell, an inner sheath cell, a hub cell, and a cyst progenitor cell.

11. The method of claim 1, wherein the gene is bam.

12. The method of claim 11, wherein the molecule expressed from the stem niche cell is a BMP polypeptide.

13. The method of claim 11 further comprising, causing the stem nitch cell to constitutively express the BMP polypeptide.

14. The method of claim 13, wherein a vector comprising a nucleic acid sequence encoding a BMP polypeptide is introduced into the stem niche cell causing the stem niche cell to constitutively express the BMP polypeptide.

15. The method of claim 14, wherein the vector comprises an inducible promoter operably linked to the nucleic acid sequence encoding the BMP polypeptide.

16. The method of claim 12, wherein the BMP polypeptide is selected from the group consisting of dpp, gbb, and scw.

17. The method of claim 12, wherein the BMP polypeptide is selected from the group consisting of BMP-2, BMP-4, BMP-5, BMP-6, BMP-7, and BMP-8.

18. The method of claim 11, wherein the signal transduction cascade comprises at least one molecule selected from the group consisting of tkv, sax, punt, pMad, Mad, Med, Dad, and shn.

19. An isolated stem cell population having at least one stem cell produced by the method of claim 1.

20. A method for maintaining the undifferentiated state of an isolated stem cell, the method comprising:
(a) providing an isolated stem cell;
(b) contacting in vitro, the stem cell with an isolated stem niche cell that expresses a BMP polypeptide such that the BMP polypeptide activates a signal transduction cascade in the stem cell causing repression of bam transcription in the stem cell.

21. The method of claim 20, wherein the stem cell is a cultured cell.

22. The method of claim 20, wherein the stem cell and stem niche cell are isolated from an organism that is either a vertebrate or an invertebrate.

23. The method of claim 22, wherein the vertebrate is a mammal.

24. The method of claim 23, wherein the mammal is a human.

25. The method of claim 22, wherein the invertebrate is an insect.

26. The method of claim 25, wherein the insect is a Drosophila.

27. The method of claim 20, wherein the stem cell is either a somatic stem cell or a germline stem cell.

28. The method of claim 20 further comprising, maintaining the stem cell in either a pluripotent state or a totipotent state.

29. The method of claim 20, wherein the stem niche cell comprises at least one somatic cell selected from the group consisting of a terminal filament cell, a cap cell, an inner sheath cell, a hub cell, and a cyst progenitor cell.

30. The method of claim 20, wherein the BMP polypeptide is selected from the group consisting of dpp, gbb, and scw.

31. The method of claim 20, wherein the BMP polypeptide is selected from the group consisting of BMP-2, BMP-4, BMP-5, BMP-6, BMP-7, and BMP-8.

32. The method of claim 20, wherein the signal transduction cascade comprises at least one molecule selected from the group consisting of tkv, sax, punt, pMad, Mad, Med, Dad, and shn.

33. The method of claim 20 further comprising, causing the stem nitch cell to constitutively express the BMP polypeptide.

34. The method of claim 33, wherein a vector comprising a nucleic acid sequence encoding a BMP polypeptide is introduced into the stem niche cell causing the stem niche cell to constitutively express the BMP polypeptide.

35. The method of claim 34, wherein the vector comprises an inducible promoter operably linked to the nucleic acid sequence encoding the BMP polypeptide.

36. The method of claim 35, wherein the BMP polypeptide is gbb.

37. An isolated stem cell population having at least one stem cell produced by the method of claim 20.

38. A method for maintaining the undifferentiated state of an isolated Drosophila germline stem cell, the method comprising:
(a) providing an isolated Drosophila germline stem cell;
(b) contacting in vitro, the germline stem cell with an isolated gbb polypeptide such that the gbb polypeptide activates a signal transduction cascade in the germline stem cell causing repression of bam transcription in the germline stem cell.

39. The method of claim 38, wherein the germline stem cell is a cultured cell.

40. The method of claim 38, wherein the germline stem cell is from an ovary.

41. The method of claim 38, wherein the germline stem cell is from a testis.

42. The method of claim 38, wherein the germline stem cell is from an embryo.

43. The method of claim 40 further comprising, contacting the isolated germline stem cell with an isolated dpp polypeptide.

44. The method of claim 38, wherein gbb polypeptide is from a source selected from the group consisting of:
(a) an isolated stem niche cell comprising at least one cell selected from the group consisting of a terminal filament cell, a cap cell, an inner sheath cell, a hub cell, and a cyst progenitor cell;
(b) an expression vector comprising a nucleic acid sequence encoding a gbb polypeptide; and
(c) a synthetic gbb molecule.

45. The method of claim 38, wherein the signal transduction cascade comprises at least one molecule selected from the group consisting of tkv, sax, punt, pMad, Mad, Med, Dad, and shn.

46. An isolated stem cell population having at least one stem cell produced by the method of claim 38.

47. A method for repressing the expression of bam in an isolated stem cell, the method comprising contacting in vitro, the stem cell with an isolated BMP polypeptide such that the BMP polypeptide activates a signal transduction cascade in the stem cell causing repression of bam transcription.

48. The method of claim 47, wherein the stem cell is a cultured cell.

49. The method of claim 47, wherein the stem cell is either a germline stem cell or a somatic stem cell.

50. The method of claim 47, wherein BMP polypeptide is from a source selected from the group consisting of:
(a) an isolated stem niche cell comprising at least one cell selected from the group consisting of a terminal filament cell, a cap cell, an inner sheath cell, a hub cell, and a cyst progenitor cell;
(b) an expression vector comprising a nucleic acid sequence encoding a BMP polypeptide; and
(c) a synthetic BMP molecule.

51. The method of claim 47, wherein the BMP polypeptide is selected from the group consisting of dpp, gbb, and scw.

52. The method of claim 47, wherein the BMP molecule is selected from the group consisting of BMP-2, BMP-4, BMP-5, BMP-6, BMP-7, and BMP-8.

53. The method of claim 47, wherein the signal transduction cascade comprises at least one molecule selected from the group consisting of tkv, sax, punt, pMad, Mad, Med, Dad, and shn.

54. A method for promoting the differentiation of a germline stem cell, the method comprising mutating at least one nucleic acid sequence encoding a participant in BMP signaling in the germline stem cell, such that the mutation results in the expression of bam in the germline stem cell.

55. The method of claim 54, wherein the method is performed either in vitro or in vivo.

56. The method of claim 54, wherein the germline stem cell is a testis cell.

57. The method of claim 54, wherein the germline stem cell is an ovary cell.

58. The method of claim 54, wherein the nucleic acid sequence mutation renders the germline stem cell non responsive to a BMP polypeptide.

59. The method of claim 54, wherein the method is performed in vivo in a Drosophila.

60. The method of claim 59, wherein the BMP polypeptide is selected from the group consisting of gbb, dpp, and scw.

61. The method of claim 54, wherein the nucleic acid sequence mutation is selected from the group consisting of frame shift, deletion, loss of function, point, and substitution.

62. The method of claim 54, wherein the nucleic acid sequence that is mutagenized is selected from the group consisting of tkv, sax, or punt nucleic acid sequence and the mutation results in loss of receptor function.

63. The method of claim 54, wherein the nucleic acid sequence that is mutagenized is selected from the group consisting of Mad, Med, or shn and the mutation substantially inhibits Mad/Med complex formation thereby preventing the complex from binding to bam.

64. A method for promoting the differentiation of a germline stem cell, the method comprising contacting a BMP polypeptide antagonist or a BMP receptor antagonist with the germline stem cell, wherein the BMP polypeptide antagonist or the BMP receptor antagonist substantially inhibits interaction of a BMP polypeptide with a BMP receptor.

65. The method of claim 64, wherein the antagonist is an antibody specific for the BMP polypeptide or the BMP receptor.

66. The method of claim 64, wherein the method is performed in vivo or in vitro.

67. The method of claim 64, wherein the BMP polypeptide is gbb.

68. A method to promote the differentiation of a germline stem cell, the method comprising introducing a vector into the germline stem cell, the vector comprising a nucleic acid sequence encoding bam, such that introduction of the vector results in an increase in the expression of bam relative to a wild type germ line stem cell.

69. The method of claim 68, wherein the method is performed in vivo or in vitro.

70. The method of claim 68, wherein the vector comprises an inducible promoter operably linked to the nucleic acid sequence encoding bam.

71. A method to regulate the expression of a germline stem cell, the method comprising introducing a vector into the germline stem cell, the vector having a nucleic acid sequence encoding bam operably linked to an inducible promoter.

72. The method of claim 71, wherein the method in performed in vivo or in vitro.

73. The method of claim 71, wherein when the promoter is induced the germline stem cell differentiates into a stem daughter cell.

74. A method to regulate a germline stem cell differentiation pathway, the pathway comprising a BMP signaling pathway, the method comprising introducing a vector into the germline stem cell, the vector having a nucleic acid sequence encoding barn operably linked to an inducible promoter.

75. The method of claim 74, wherein the method is performed in vivo or in vitro.

76. The method of claim 74, wherein when the promoter is induced the germline stem cell differentiates into a stem daughter cell.

77. A method to regulate a germline stem cell differentiation pathway, the pathway comprising a BMP signaling pathway, the method comprising mutagenizing a nucleic acid sequence encoding a participant of the BMP signaling pathway in the germline stem cell.

78. The method of claim 77, wherein the method is performed in vitro or in vivo.

79. The method of claim 77, wherein the participant of the BMP signaling pathway is selected from the group consisting of tkv, sax, punt, pMad, Mad, Med, Dad, and shn.

80. The method of claim 77, wherein nucleic acid sequence encoding the participant of the BMP signaling pathway is mutagenized by a mutation method selected from the group consisting of frame shift, deletion, loss of function, point, and substitution.

81. A germline stem cell population having germline stem cells that have been mutagenized such that the germline stem cells are non responsive to a gbb polypeptide, thereby resulting in the expression of bar by a substantial number of germline stem cells in the population.

82. The germline stem cell population of claim 81, wherein the germline stem cells are mutagenized such that a receptor selected from the group consisting of tkv, sax, and punt has a loss of receptor function.

83. The germline stem cell population of claim 81, wherein the germline stem cells are mutagenized such that Mad, Med, and shn are prevented from forming a Mad/Med complex.

84. The germline stem cell population of claim 81, wherein the germline stem cells are testis cells.

85. The germline stem cells of claim 81, wherein the germline stem cells are ovary cells.

86. The germline stem cells of claim 81, wherein the germline stem cells are maintained either in vitro or in vivo.

87. The germline stem cells of claim 81, wherein the geimline stem cells divide symmetrically or asymmetrically.

88. The germline stem cells of claim 81, wherein the germline stem cells are from an invertebrate or a vertebrate.

89. The method of claim 88, wherein the vertebrate is a mammal.

90. The method of claim 88, wherein the invertebrate is a Drosophila.

91. A germline cell population, the germline cell population comprising germline stem cells and germline stem niche cells, the germline stem niche cells having an introduced vector comprising a nucleic acid encoding a gbb polypeptide operatively linked to an inducible promoter, such that the germline stem niche cells overexpress the gbb polypeptide when the promoter is induced, thereby resulting in the repression of bam transcription by a substantial number of germline stem cells in the population.

92. The germline cell population of claim 91, wherein the germline cells are testis cells.

93. The germline cell population of claim 91, wherein the germline cells are ovary cells.

94. The germline cell population of claim 91, wherein the germline stem cells are maintained either in vitro or in vivo.

95. The germline cell population of claim 91, wherein the germline stem cells divide symmetrically or asymmetrically.

96. The germline cell population of claim 91, wherein the germline cell are from an invertebrate or a vertebrate.

97. The method of claim 96, wherein the vertebrate is a mammal.

98. The method of claim 96, wherein the invertebrate is a Drosophila.

99. A germline cell population, the germline cell population comprising germline stem cells and germline stem niche cells, the germline stem niche cells having a gbb nucleic acid sequence that has been mutagenized such that the expressed gbb polypeptide is inhibited from interacting with a BMP receptor on the germline stem cells, thereby resulting in the expression of bam by a substantial number of germline stem cells in the population.

100. The germline cell population of claim 99, wherein the gbb nucleic acid sequence mutation is selected from the group consisting of frame shift, point substitution, loss of function, knock-out deletion, and deletion mutation.

101. The germline stem cell of claim 99, wherein the BMP receptor is selected from the group consisting of tkv, sax, and punt.

102. The germline stem cell of claim 99, wherein Mad, Med, and shn are prevented from forming a Mad/Med complex.

103. In vivo testicular tissue comprising gbb mutant clonal stem niche cells, wherein the stem niche cells express substantially more gbb polypeptide compared to a stem niche cell in wild type testicular tissue.

104. The tissue of claim 103, wherein the stem niche cells are selected from the group consisting of a terminal filament cell, a cap cell, an inner sheath cell, a hub cell, and a cyst progenitor cell.

105. The testicular tissue of claim 103 wherein the tissue has an increased number of germline stem cells compared to wild type testicular tissue.

106. The testicular tissue of claim 103, wherein the tissue is from a vertebrate or an invertebrate.

107. The testicular tissue of claim 106, wherein the vertebrate is a mammal.

108. The testicular tissue of claim 106, wherein the invertebrate is a Drosophila.

109. The testicular tissue of claim 103, wherein the cells comprising the tissue divide symmetrically and asymmetrically.

110. The testicular tissue of claim 103, wherein the stem niche cells express substantially more gbb polypeptide as a result of introducing a vector into the stem niche cells, the vector having a nucleic acid encoding a gbb polypeptide operably linked to an inducible promoter.

111. In vivo testicular tissue comprising gbb mutant clonal stem niche cells, wherein the stem niche cells express substantially less gbb polypeptide compared to a stem niche cell in wild type testicular tissue.

112. The tissue of claim 111, wherein the stem niche cell are selected from the group consisting of a terminal filament cell, a cap cell, an inner sheath cell, a hub cell, and a cyst progenitor cell.

113. The testicular tissue of claim 111, wherein the tissue has a decreased number of germline stem cells compared to wild type testicular tissue.

114. The testicular tissue of claim 111, wherein the tissue is from a vertebrate or an invertebrate.

115. The testicular tissue of claim 114, wherein the vertebrate is a mammal.

116. The testicular tissue of claim 114, wherein the invertebrate is a Drosophila.

117. The testicular tissue of claim 115, wherein the cells comprising the tissue divide symmetrically and asymmetrically.

118. The testicular tissue of claim 114, wherein the stem niche cells express substantially less gbb polypeptide as a result mutagenizing gbb nucleic acid sequence in the stem niche cells.

119. The testicular tissue of claim 118, wherein the gbb nucleic acid sequence is mutagenized by a mutation method selected from the group consisting of frame shift, loss of function, point, deletion, and substitution.

120. An in vitro germline stem cell cultivation system, comprising:
(a) isolated germline tissue, the tissue having germline stem cells that have been mutagenized such that the germline stem cells are non responsive to a gbb polypeptide; and
(b) a culture medium.

121. The germline stem cell cultivation system of claim 123, wherein the germline tissue is testicular tissue isolated from Drosophila.

122. An in vitro germline stem cell cultivation system, comprising:
(a) isolated germline tissue, the tissue comprising stem cells and stem niche cells, wherein the stem niche cells have been mutated so that they express substantially more gbb polypeptide compared to stem niche cells in wild type germline tissue; and
(b) a culture medium.

123. The germline stem cell cultivation system of claim 125, wherein the germline tissue is testicular tissue isolated from Drosophila.

124. An in vitro germline stem cell cultivation system, comprising:
(a) isolated germline tissue, the tissue comprising stem cells and stem niche cells, wherein the stem niche cells have been mutated so that they express substantially less gbb polypeptide compared to stem niche cells in wild type germline tissue; and
(b) a culture medium.

125. The germline stem cell cultivation system of claim 124, wherein the germline tissue is testicular tissue isolated from Drosophila.

126. A marker to detect germline stem cell differentiation, the marker comprising gbb.

127. The germline stem cell cultivation system of claim 126, wherein the germline tissue is ovary tissue isolated from Drosophila.

128. The germline stem cell cultivation system of claim 122, wherein the germline tissue is ovary tissue isolated from Drosophila.

129. The germline stem cell cultivation system of claim 120, wherein the germline tissue is ovary tissue isolated from Drosophila.
